(19) United States
(12) Patent Application Publication
(10) Pub. No.: US 2011/0165568 A1
(43) Pub. Date: Jul. 7, 2011

(54) SEQUENCES OF E.COLI 055:H7 GENOME

(75) Inventors: Paolo Vatta, San Mateo, CA (US); Pius Brzoska, Woodside, CA (US); Elizabeth Newton, Foster City, CA (US); Melissa Barker, Gilroy, CA (US); Olga V. Petrauskene, San Carlos, CA (US); Manohar Furtado, San Ramon, CA (US); Craig Cummings, Pacifica, CA (US)

(73) Assignee: LIFE TECHNOLOGIES CORPORATION, Carlsbad, CA (US)

(21) Appl. No.: 12/981,795

(22) Filed: Dec. 30, 2010

Related U.S. Application Data
(60) Provisional application No. 61/291,652, filed on Dec. 31, 2009.
(60) Provisional application No. 61/291,662, filed on Dec. 31, 2009.
(60) Provisional application No. 61/292,438, filed on Jan. 5, 2010.

(51) Int. Cl. C12Q 1/68; C07H 21/04

(52) U.S. Cl. 435/6.11; 536/23.1

(57) ABSTRACT

Disclosed is the genomic sequence for E. coli O55:H7 as well as compositions, methods, and kits for detecting, identifying and distinguishing E. coli O55:H7 from non-O55:H7 strains. In some embodiments, isolated nucleic acid compositions unique and/or specific to E. coli O55:H7 are described. Methods of detection and/or indentifying E. coli O55:H7 comprising detecting at least one nucleic acid sequences comprising or derived from SEQ ID NO:1-5, SEQ ID NO: 66, SEQ ID NO: 252, SEQ ID NO: 1113, and SEQ ID NO: 1461, are described. Primer and probe compositions and methods of use of primers and probes are also provided. Kits for identification of E. coli O55:H7 are also described.

### CROSS-REFERENCE TO RELATED APPLICATIONS

This application claims the benefit under 35 U.S.C. §119(e) of U.S. Provisional Patent Application U.S. Ser. No. 61/291,652 filed Dec. 31, 2009; U.S. Provisional Patent Application U.S. Ser. No. 61/291,662 filed Dec. 31, 2009; and U.S. Provisional Patent Application U.S. Ser. No. 61/292,438 filed Jan. 5, 2010; the entire contents of which are incorporated herein by reference.

### EFS INCORPORATION PARAGRAPH RELATING TO SEQUENCE LISTING

The instant application contains a Sequence Listing which has been submitted in ASCII format via EFS-WEB and is hereby incorporated by reference in its entirety. Said ASCII copy, created on Dec. 27, 2010, is named LT0078US.TXT and is 14,312,068 bytes in size.

### FIELD

The present teachings relate to compositions, methods and kits for detection and identification of Escherichia coli (E. coli) O55:H7. More particularly, the specification describes compositions and kits comprising nucleic acid sequences specific and/or unique to E. coli O55:H7 and methods of use thereof. Methods for differentially detecting E. coli O55:H7 from other pathogens (including closely related serotypes such as E. coli O157:H7) are also described.

### BACKGROUND

Escherichia coli O55:H7 is a serotype of E. coli that is occasionally associated with hemorrhagic diarrhea and infantile diarrhea in humans. E. coli O55:H7 is thought to be harbored in the digestive tract of cattle and therefore has the potential to enter the food supply.

E. coli O55:H7 is very closely related to a pathogenic serotype of E. coli, E. coli O157:H7, a causative agent of enterohemorrhagic colitis and hemorrhagic uremic syndrome in humans. E. coli O157:H7 has been identified by the United States Department of Agriculture (USDA) as a pathogen required to be tested while determining food safety. E. coli O157:H7 appears to have evolved stepwise from E. coli O55:H7. These two serotypes are more closely related at the nucleotide level while divergence is markedly different at the gene level. Likewise, other E. coli serotypes have been shown to be less divergent at the nucleotide level making identification of pathogenic strains difficult. Most assays that target E. coli O157:H7 also detect E. coli O55:H7. Furthermore, designing assays specific for E. coli O157:H7 has been difficult due to the absence of genomic information regarding its closest relative, E. coli O55:H7.

Design and development of molecular detection assays that differentiate or identify a target sequence that is present in organisms to be detected, and absent or divergent in organisms not to be detected is an unmet need for the definitive detection of the pathogenic O157:H7 serotype of E. coli.

### SUMMARY OF SOME EMBODIMENTS OF THE DISCLOSURE

The present disclosure, in some embodiments, discloses the complete genomic sequence of an E. coli O55:H7. In some embodiments, the disclosure describes isolated nucleic acid sequence compositions comprising portions of an E. coli O55:H7 genome. In some embodiments, isolated nucleic acid sequence compositions of the disclosure comprise nucleic acid sequences unique to and/or specific to an E. coli O55:H7 organism. In some embodiments, isolated nucleic acid sequences of the disclosure may have at least 90% sequence identity, at least 80% sequence identity, and/or at least 70% sequence identity to nucleic acid sequences comprising unique and/or specific portions of an E. coli O55:H7 genome.

In some embodiments, unique E. coli O55:H7 nucleic acid sequences may comprise isolated nucleic acid molecules comprising a nucleotide sequence of SEQ ID NO:66, SEQ ID NO:252, SEQ ID NO:1113, SEQ ID NO:1461, SEQ ID NO:1, SEQ ID NO:2, SEQ ID NO:3, SEQ ID NO:4, SEQ ID NO:5, fragments thereof, and/or complements thereof. In some embodiments, unique E. coli O55:H7 sequences may comprise isolated nucleic acid molecules comprising a nucleotide sequence having at least a 90% sequence identity, at least 80% sequence identity and/or at least 70% sequence identity to the nucleotide sequences of SEQ ID NO:66, SEQ ID NO:252, SEQ ID NO:1113, SEQ ID NO:1461, SEQ ID NO:1, SEQ ID NO:2, SEQ ID NO:3, SEQ ID NO:4, SEQ ID NO:5, fragments thereof and/or complements thereof.

In some embodiments, E. coli O55:H7 isolated nucleic acid sequences may comprise nucleic acid molecules comprising at least 40 nucleotide sequence of SEQ ID NO:66, SEQ ID NO:252, SEQ ID NO:1113, SEQ ID NO:1461, SEQ ID NO:1, SEQ ID NO:2, SEQ ID NO:3, SEQ ID NO:4, SEQ ID NO:5; at least 30 nucleotide sequence of SEQ ID NO:66, SEQ ID NO:252, SEQ ID NO:1113, SEQ ID NO:1461, SEQ ID NO:1, SEQ ID NO:2, SEQ ID NO:3, SEQ ID NO:4, SEQ ID NO:5; at least 25 nucleotide sequence of SEQ ID NO:66, SEQ ID NO:252, SEQ ID NO:1113, SEQ ID NO:1461, SEQ ID NO:1, SEQ ID NO:2, SEQ ID NO:3, SEQ ID NO:4, SEQ ID NO:5; at least 20 nucleotide sequence of SEQ ID NO:66, SEQ ID NO:252, SEQ ID NO:1113, SEQ ID NO:1461, SEQ ID NO:1, SEQ ID NO:2, SEQ ID NO:3, SEQ ID NO:4, SEQ ID NO:5; at least 15 nucleotide sequence of SEQ ID NO:66, SEQ ID NO:252, SEQ ID NO:1113, SEQ ID NO:1461, SEQ ID NO:1, SEQ ID NO:2, SEQ ID NO:3, SEQ ID NO:4, SEQ ID NO:5; at least 10 nucleotide sequence of SEQ ID NO:66, SEQ ID NO:252, SEQ ID NO:1113, SEQ ID NO:1461, SEQ ID NO:1, SEQ ID NO:2, SEQ ID NO:3, SEQ ID NO:4, SEQ ID NO:5; any intermediate number of contiguous sequences from at least about 10 nucleotides of sequence to at least about 40 nucleotides of sequence of SEQ ID NO:66, SEQ ID NO:252, SEQ ID NO:1113, SEQ ID NO:1461, SEQ ID NO:1, SEQ ID NO:2, SEQ ID NO:3, SEQ ID NO:4, SEQ ID NO:5 and sequences having 90% identity to the foregoing sequences.

In some embodiments, the disclosure describes compositions of isolated nucleic acid sequences having SEQ ID NO: 6, SEQ ID NO:7, SEQ ID NO:8, SEQ ID NO:9, SEQ ID NO:10, SEQ ID NO: 11, SEQ ID NO:12, SEQ ID NO:13, SEQ ID NO:14, SEQ ID NO:15, SEQ ID NO:16, SEQ ID NO: 17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, fragments thereof, at least 10 contiguous nucleotide sequences thereof, complements thereof and isolated nucleic acid sequence comprising at least 90% nucleic acid sequence identity to the sequences set forth above.

In some embodiments, isolated nucleic acid sequence compositions of the disclosure may further comprise one or more label, such as, but not limited to, a dye, a radioactive isotope, a chemiluminescent label, a fluorescent moiety, a bioluminescent label an enzyme, and combinations thereof.

The disclosure also describes recombinant constructs comprising nucleic acid sequences unique to E. coli O55:H7 as set forth in sections above. Accordingly, a recombinant construct of the disclosure may comprise a nucleotide sequence of SEQ ID NO:66, SEQ ID NO:252, SEQ ID NO:1113, SEQ ID NO:1461, SEQ ID NO:1, SEQ ID NO:2, SEQ ID NO:3, SEQ ID NO:4, SEQ ID NO:5, fragments thereof, complements thereof as well as nucleotide sequences having at least a 90% identity, at least 80% identity and/or at least 70% identity to the nucleotide sequences described above. In some embodiments, a recombinant construct of the disclosure may comprise a nucleotide sequence of SEQ ID NO: 6, SEQ ID NO:7, SEQ ID NO:8, SEQ ID NO:9, SEQ ID NO:10, SEQ ID NO: 11, SEQ ID NO:12, SEQ ID NO:13, SEQ ID NO:14, SEQ ID NO:15, SEQ ID NO:16, SEQ ID NO: 17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, fragments thereof, at least 10 contiguous nucleotide sequences thereof, complements thereof and isolated nucleic acid sequence comprising at least 90% nucleic acid sequence identity to the sequences set forth above.

The specification also discloses methods for detection of an E. coli O55:H7 organism from a sample and methods to exclude the presence of an E. coli O55:H7 organism in a sample, wherein the detection of at least one nucleic acid sequence that is unique to an E. coli O55:H7 is indicative of the presence of an E. coli O55:H7 and the absence of detection of any nucleic acid sequence unique to an E. coli O55:H7 is indicative of the absence of an E. coli O55:H7 in the sample. Accordingly, a method of the disclosure, in some embodiments, may comprise detecting, in a sample, a nucleic acid sequence having at least 10 to at least 25 nucleic acids of SEQ ID NO:66, SEQ ID NO:252, SEQ ID NO:1113, SEQ ID NO:1461, SEQ ID NO:1, SEQ ID NO:2, SEQ ID NO:3, SEQ ID NO:4, SEQ ID NO:5, and/or complementary sequences thereof, wherein detection of the nucleic acid sequence indicates the presence of an E. coli O55:H7 organism in the sample. Methods of detection may also comprise identification steps and may further comprise steps of sample preparation. Such embodiments are described in detail in sections below.

Some embodiments describe methods of distinguishing an E. coli O55:H7 from a non-O55:H7 E. coli strains and may comprise: detecting at least one of a nucleic acid sequence having a nucleic acid sequence of SEQ ID NO: 66, SEQ ID NO:252, SEQ ID NO:1113, SEQ ID NO:1461, SEQ ID. NO: 1, SEQ ID NO: 2, SEQ ID NO: 3, SEQ ID NO:4, SEQ ID NO:5, fragments thereof, complements thereof and/or sequences comprising at least 90% nucleic acid sequence identity thereof, wherein detection of one of the at least one nucleic acid sequences identifies E. coli O55:H7. In other embodiments, not detecting at least one of a nucleic acid sequence selected from nucleotides described by either SEQ ID NO:66, SEQ ID NO:252, SEQ ID NO:1113, SEQ ID NO:1461, SEQ ID. NO: 1, SEQ ID NO: 2, SEQ ID NO: 3, SEQ ID NO:4, SEQ ID NO:5, fragments thereof, complements thereof and/or sequences comprising at least 90% nucleic acid sequence identity thereof may be used to exclude the presence of E. coli O55:H7 in a sample.

Some methods for identifying and/or detecting E. coli O55:H7 in a sample may comprise using a nucleotide sequence composition of the disclosure for detection. Exemplary compositions of the disclosure used for detection methods may comprise, but are not limited to, SEQ ID NO: 6, SEQ ID NO:7, SEQ ID NO:8, SEQ ID NO:9, SEQ ID NO:10, SEQ ID NO: 11, SEQ ID NO:12, SEQ ID NO:13, SEQ ID NO:14, SEQ ID NO:15, SEQ ID NO:16, SEQ ID NO: 17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, fragments thereof, at least 10 contiguous nucleotide sequences thereof, complements thereof, isolated nucleic acid sequence comprising at least 90% nucleic acid sequence identity to the sequences set forth above and/or labeled derivatives thereof.

Some embodiments of the present disclosure are kits for detection of E. coli O55:H7. A kit of the disclosure may comprise one or more isolated nucleic acid sequences of the disclosure as set forth herein. Some nucleic acid compositions of the disclosure may comprise primers for amplification of target nucleic acid sequences from a contaminating E. coli O55:H7 that may be present in a sample. Some nucleic acid compositions of the disclosure may comprise probes for the detection of target nucleic acid sequences and/or amplified target nucleic acid regions from a contaminating E. coli O55:H7 present in a sample. Probes and primers comprised in kits may be labeled. Kits may additionally comprise one or more components such as, but not limited to: buffers, enzymes, nucleotides, salts, reagents to process and prepare samples, probes, primers, agents to enable detection and control nucleotides. Each component of a kit of the disclosure may be packaged individually or together in various combinations in one or more suitable container means. Kits of the disclosure, in some embodiments, may be used to distinguish the presence of non-O55:H7 bacteria.

It is a feature of the embodiments disclosed herein that a subject bacterium, referred to as E. coli O55:H7 (Applied Biosystems, collection designation, PE704), has been deposited with the American Type Culture Collection (ATCC) on Jul. 23, 2009 and has the ATCC designation number PTA-10235.

### BRIEF DESCRIPTION OF THE DRAWINGS

Some specific example embodiments of the disclosure may be understood by referring, in part, to the following description and the accompanying drawings, wherein:

FIG. 1 is a table that depicts exemplary E. coli O55:H7 specific and unique nucleic acid sequences.

FIG. 2 is a plot of SNP density in 1 Kb windows across an E. coli O55:H7 genome.

FIG. 3 lists and describes a few selected identified open reading frames in an E. coli O55:H7 pseudochromosome (pseudochromosome sequence is comprised in SEQ ID NO 1695 in the attached Sequence Listing).

### DETAILED DESCRIPTION OF EXEMPLARY EMBODIMENTS

For purposes of interpreting this specification, the following definitions will apply and whenever appropriate, terms used in the singular will also include the plural and vice versa. In the event that any definition set forth below conflicts with the usage of that word in any other document, including any document incorporated herein by reference, the definition set forth below shall always control for purposes of interpreting this specification and its associated claims unless a contrary meaning is clearly intended (for example in the document where the term is originally used). It is noted that, as used in this specification and the appended claims, the singular forms “a,” “an,” and “the,” include plural referents unless expressly and unequivocally limited to one referent. The use of “or” means “and/or” unless stated otherwise. For illustration purposes, but not as a limitation, “X and/or Y” can mean “X” or “Y” or “X and Y”. The use of “comprise,” “comprises,” “comprising,” “having,” “include,” “includes,” and “including” are interchangeable and open terms not intended to be limiting. Furthermore, where the description of one or more embodiments uses the term “comprising,” those skilled in the art would understand that, in some specific instances, the embodiment or embodiments can be alternatively described using the language “consisting essentially of” and/or “consisting of”. The term “and/or” means one or all of the listed elements or a combination of any two or more of the listed element.

The section headings used herein are for organizational purposes only and are not to be construed as limiting the described subject matter in any way. All literature cited in this specification, including but not limited to, patents, patent applications, articles, books, and treatises are expressly incorporated by reference in their entirety for any purpose. In the event that any of the incorporated literature contradicts any term defined herein, this specification controls. While the present teachings are described in conjunction with various embodiments, it is not intended that the present teachings be limited to such embodiments. On the contrary, the present teachings encompass various alternatives, modifications, and equivalents, as will be appreciated by those of skill in the art.

The practice of the present embodiments may employ conventional techniques and descriptions of organic chemistry, polymer technology, molecular biology (including recombinant techniques), cell biology, biochemistry, and immunology, which are within the skill of the art, in light of the present teachings. Some conventional techniques include, but may not be limited to, oligonucleotide synthesis, hybridization, extension reactions and detection of hybridization using a label. Specific illustrations of suitable techniques may be described in example herein below. However, other equivalent conventional procedures may also be used. General conventional techniques and their descriptions can be found in standard laboratory manuals such as Genome Analysis: A Laboratory Manual Series (Vols. I-IV), PCR Primer: A Laboratory Manual, and Molecular Cloning: A Laboratory Manual (all from Cold Spring Harbor Laboratory Press, 1989), Gait, “Oligonucleotide Synthesis: A Practical Approach” 1984, IRL Press, London, Nelson and Cox (2000), Lehninger, Principles of Biochemistry 3^{rd} Ed., W. H. Freeman Pub., New York, N.Y. and Berg et al. (2002) Biochemistry, 5^{th} Ed., W. H. Freeman Pub., New York, N.Y. all of which are herein incorporated in their entirety by reference for all purposes.

The terms “amplifying” and “amplification” are used in a broad sense and refer to any technique by which a target region, an amplicon, or at least part of an amplicon, is reproduced or copied (including the synthesis of a complementary strand), typically in a template-dependent manner, including a broad range of techniques for amplifying nucleic acid sequences, either linearly or exponentially. Some non-limiting examples of amplification techniques include primer extension, including the polymerase chain reaction (PCR), reverse transcription polymerase chain reaction (RT-PCR), asynchronous PCR (A-PCR), and asymmetric PCR (AM-PCR), strand displacement amplification (SDA), multiple displacement amplification (MDA), nucleic acid strand-based amplification (NASBA), rolling circle amplification (RCA), transcription-mediated amplification (TMA), and the like, including multiplex versions, and combinations thereof. Descriptions of certain amplification techniques can be found in, among other places, Molecular Cloning, A Laboratory Manual, Cold Spring Harbor Press, 3d ed., 2001 (hereinafter “Sambrook and Russell”); Sambrook et al.; Ausubel et al.; PCR Primer: A Laboratory Manual, Diffenbach, Ed., Cold Spring Harbor Press (1995); Msuih et al., J. Clin. Micro. 34:501-07 (1996); McPherson; Rapley; U.S. Pat. Nos. 6,027,998 and 6,511,810; PCT Publication Nos. WO 97/31256 and WO 01/92579; Ehrlich et al., Science 252:1643-50 (1991); Favis et al., Nature Biotechnology 18:561-64 (2000); Protocols & Applications Guide, rev. 9/04, Promega, Madison, Wis.; and Rabenau et al., Infection 28:97-102 (2000).

The terms “amplicon,” “amplification product” and “amplified sequence” are used interchangeably herein and refer to a broad range of techniques for increasing polynucleotide sequences, either linearly or exponentially and can be the product of an amplification reaction. An amplicon can be double-stranded or single-stranded, and can include the separated component strands obtained by denaturing a double-stranded amplification product. In certain embodiments, the amplicon of one amplification cycle can serve as a template in a subsequent amplification cycle. Exemplary amplification techniques include, but are not limited to, PCR or any other method employing a primer extension step. Other nonlimiting examples of amplification include, but are not limited to, ligase detection reaction (LDR) and ligase chain reaction (LCR). Amplification methods can comprise thermal-cycling or can be performed isothermally. In various embodiments, the term “amplification product” and “amplified sequence” includes products from any number of cycles of amplification reactions.

As used herein, the term “analyzing” refers to evaluating and comparing the results of a method. In some exemplary embodiments, “analyzing” refers to evaluating and comparing the results of a sample tested to a second sample and/or to a control in a method of the disclosure.

As used herein, “complement” and “complements” are used interchangeably and refer to the ability of a nucleotide, a polynucleotide or two single stranded polynucleotides (for instance, a primer and a target polynucleotide) to base pair with each other, where an adenine on one strand of a polynucleotide will base pair to a thymine or uracil on a strand of a second polynucleotide and a cytosine on one strand of a polynucleotide will base pair to a guanine on a strand of a second polynucleotide. Two polynucleotides are complementary to each other when a nucleotide sequence in one polynucleotide can base pair with a nucleotide sequence in a second polynucleotide. For instance, 5′-ATGC-3′ and 5′-GCAT-3′ are complementary.

As used herein the term “complementary nucleotide sequence” and “complementary sequences” refers to a (second) nucleotide sequence which, by base pairing, is the complement of a first nucleotide sequence. For example, a forward strand with the sequence 5′-ATGGC-3′ would have the complementary nucleotide sequence 3′-TACCG-5′, also termed the “reverse strand.”

As used herein, the term “contacting” as used herein refers to the hybridization between a primer and its substantially complementary region. “Contacting” may also refer to bringing in contact at least two moieties (reagents, cells, nucleic acids) to bring about a change or a reaction in one or all the moieties. The process of contacting may also comprise “incubating” (contacting for a certain time lengths) and/or incubating at certain temperatures to bring about the change or reaction.

As used herein, “DNA” refers to deoxyribonucleic acid in its various forms as understood in the art, such as genomic DNA, cDNA, isolated nucleic acid molecules, vector DNA, and chromosomal DNA. “Nucleic acid” refers to DNA or RNA in any form. Examples of isolated nucleic acid molecules include, but are not limited to, recombinant DNA molecules contained in a vector, recombinant DNA molecules maintained in a heterologous host cell, partially or substantially purified nucleic acid molecules, and synthetic DNA molecules. Typically, an “isolated” nucleic acid is free of sequences which naturally flank the nucleic acid (i.e., sequences located at the 5′ and 3′ ends) in the native nucleic acid or genomic DNA of the organism from which the nucleic acid is derived. Moreover, an “isolated” nucleic acid molecule, such as a cDNA molecule, is generally substantially free of other cellular material when isolated from a cell and/or culture medium when produced by recombinant techniques, and/or substantially free of chemical precursors or other chemicals when chemically synthesized.

The terms “detecting” and “detection” are used in a broad sense herein and encompass any technique by which one can determine the absence or presence of something, and/or identify a nucleic acid sequence and/or a protein encoded by a nucleic acid sequence. In some embodiments, detecting comprises quantitating a detectable signal from the nucleic acid, including without limitation, a real-time detection method, such as quantitative PCR (“Q-PCR”). In some embodiments, detecting comprises determining the sequence of a sequencing product or a family of sequencing products generated using an amplification product as the template; in some embodiments, such detecting comprises obtaining the sequence of a family of sequencing products.

As used here, “distinguishing” and “distinguishable” are used interchangeably and refer to differentiating between at least two results from substantially similar or identical reactions, including but not limited to, two different amplification products, two different melting temperatures, two different melt curves, and the like. The results can be from a single reaction, two reactions conducted in parallel, two reactions conducted independently, i.e., separate days, operators, laboratories, and so on.

As used herein, the term “E. coli O55:H7-specific nucleotide sequence” and “a nucleic acid sequence unique to E. coli O55:H7” refers broadly to nucleotide sequences specific and/or unique to E. coli O55:H7 and not known or found in other E. coli strains or in other related and/or unrelated microorganisms. These include, but are not limited to, nucleic acid sequences comprised in SEQ ID NO: 66, SEQ ID NO:252, SEQ ID NO:1113, SEQ ID NO:1461, SEQ ID NO:1, SEQ ID NO:2, SEQ ID NO:3, SEQ ID NO:4, SEQ ID NO:5, as well as fragments, complements, and sequences having at least 90% sequence identity thereof.

As used herein, the term “homology” refers to a degree of complementarity at the nucleic acid level that can be determined by known methods, e.g. computer-assisted sequence comparisons (Basic local alignment search tool, S. F. Altschul et al., J. Mol. Biol. 215 (1990), 403 410). The term “homology” known to the skilled person describes the degree to which two or more nucleic acid molecules are related, this being determined by the concordance between the sequences. The percentage of “homology” is obtained from the percentage of identical regions in two or more sequences, taking into account gaps or other sequence peculiarities. The homology of nucleic acid molecules which are related to one another can be determined with the aid of known methods. As a rule, special computer programs with algorithms which take account of the particular requirements are employed. There can be partial homology or complete homology (i.e., identity). A partially complementary sequence that at least partially inhibits a completely complementary sequence from hybridizing to a target nucleic acid is referred to using the functional term “substantially homologous.”

The term “selectively hybridize” and variations thereof means that under appropriate stringency conditions, a given sequence (for example, but not limited to, a primer) anneals with a second sequence comprising a complementary string of nucleotides (for example but not limited to a target flanking sequence or a primer-binding site of an amplicon), but does not anneal to undesired sequences, such as non-target nucleic acids or other primers. Typically, as the reaction temperature increases toward the melting temperature of a particular double-stranded sequence, the relative amount of selective hybridization generally increases and mis-priming generally decreases. In this specification, a statement that one sequence hybridizes or selectively hybridizes with another sequence encompasses situations where the entirety of both of the sequences hybridize to one another and situations where only a portion of one or both of the sequences hybridizes to the entire other sequence or to a portion of the other sequence.

The terms “identity”, “nucleic acid sequence identity” and “sequence identity” are used interchangeably and refer to the percentage of pair-wise identical residues—following homology alignment of a sequence of a polynucleotide with a sequence in question—with respect to the number of residues in the longer of these two sequences. The term “identity” as known in the art refers to a relationship between the sequences of two or more polypeptide molecules or two or more nucleic acid molecules, as determined by comparing the sequences. In the art, “identity” also means the degree of sequence relatedness between nucleic acid molecules or polypeptides, as the case may be, as determined by the match between strings of two or more nucleotide or two or more amino acid sequences. “Identity” measures the percent of identical matches between the smaller of two or more sequences with gap alignments (if any) addressed by a particular mathematical model or computer program (i.e., “algorithms”).

The term “percent (%) nucleic acid sequence identity” with respect to a nucleic acid sequence refers to the percentage of nucleotides in a first sequence that are identical with the nucleotides in a second nucleic acid sequence of interest, after aligning the sequences and introducing gaps, if necessary, to achieve the maximum percent sequence identity. Alignment for purposes of determining percent nucleic acid sequence identity can be achieved in various ways that are known to one of skill in the art, for instance, using publicly available computer software such as BLAST, BLAST-2, ALIGN or Megalign (DNASTAR) software.

Percent nucleic acid sequence identity may also be determined using the sequence comparison program NCBI-BLAST2 (Altschul et al., Nucleic Acids Res. 25:3389-3402 (1997)). The NCBI-BLAST2 sequence comparison program may be downloaded from http://www.ncbi.nlm.nih.gov or otherwise obtained from the National Institute of Health, Bethesda, Md. NCBI-BLAST2 uses several search parameters, wherein all of those search parameters are set to default values including, for example, unmask=yes, strand=all, expected occurrences=10, minimum low complexity length=15/5, multi-pass e-value=0.01, constant for multi-pas s=25, dropoff for final gapped alignment=25 and scoring matrix=BLOSUM62.

In situations where NCBI-BLAST2 is employed for sequence comparisons, the % nucleic acid sequence identity of a given nucleic acid sequence C to, with, or against a given nucleic acid sequence D (which can alternatively be phrased as a given nucleic acid sequence C that has or comprises a certain % nucleic acid sequence identity to, with, or against a given nucleic acid sequence D) is calculated as follows: 100 times the fraction W/Z where W is the number of nucleotides scored as identical matches by the sequence alignment program NCBI-BLAST2 in that program's alignment of C and D, and where Z is the total number of nucleotides in D. It will be appreciated that where the length of nucleic acid sequence C is not equal to the length of nucleic acid sequence D, the % nucleic acid sequence identity of C to D will not equal the % nucleic acid sequence identity of D to C.

The term “label” refers to any moiety which can be attached to a molecule and: (i) provides a detectable signal; (ii) interacts with a second label to modify the detectable signal provided by the second label, e.g. FRET; (iii) stabilizes hybridization, i.e. duplex formation; or (iv) provides a capture moiety, i.e. affinity, antibody/antigen, ionic complexation. Labelling can be accomplished using any one of a large number of known techniques employing known labels, linkages, linking groups, reagents, reaction conditions, and analysis and purification methods. Labels include light-emitting compounds which generate a detectable signal by fluorescence, chemiluminescence, or bioluminescence (Kricka, L. in Nonisotopic DNA Probe Techniques (1992), Academic Press, San Diego, pp. 3-28). Another class of labels comprise hybridization-stabilizing moieties which serve to enhance, stabilize, or influence hybridization of duplexes, e.g. intercalators, minor-groove binders, and cross-linking functional groups (Blackburn, G. and Gait, M. Eds. “DNA and RNA structure” in Nucleic Acids in Chemistry and Biology, 2^{nd} Edition, (1996) Oxford University Press, pp. 15-81). Yet another class of labels effect the separation or immobilization of a molecule by specific or non-specific capture, for example biotin, digoxigenin, and other haptens (Andrus, A. “Chemical methods for 5′ non-isotopic labeling of PCR probes and primers” (1995) in PCR 2: A Practical Approach, Oxford University Press, Oxford, pp. 39-54). A label may include but is not limited to a dye, a radioactive isotope, a chemiluminescent label, a fluorescent moiety, a bioluminescent moiety, and/or an enzyme.

As used herein, the terms “polynucleotide”, “oligonucleotide”, and “nucleic acid sequences” are used interchangeably and refer to single-stranded and double-stranded polymers of nucleotide monomers, including without limitation 2′-deoxyribonucleotides (DNA) and ribonucleotides (RNA) linked by internucleotide phosphodiester bond linkages, or internucleotide analogs, and associated counter ions, e.g., H⁺, NH₄⁺, trialkylammonium, Me⁺, Na⁺, and the like. A polynucleotide may be composed entirely of deoxyribonucleotides, entirely of ribonucleotides, or chimeric mixtures thereof and can include nucleotide analogs. The nucleotide monomer units may comprise any nucleotide or nucleotide analog. Polynucleotides typically range in size from a few monomeric units, e.g. 5-40 when they are sometimes referred to in the art as oligonucleotides, to several thousands of monomeric nucleotide units. Unless denoted otherwise, whenever a polynucleotide sequence is represented, it will be understood that the nucleotides are in 5′ to 3′ order from left to right and that “A” denotes deoxyadenosine, “C” denotes deoxycytosine, “G” denotes deoxyguanosine, “T” denotes thymidine, and “U” denotes deoxyuridine, unless otherwise noted.

As used herein, the terms “target polynucleotide,” “nucleic acid target” and “target nucleic acid” are used interchangeably and refer to a particular nucleic acid sequence of interest. The “target” can be a polynucleotide sequence that is sought to be amplified and can exist in the presence of other nucleic acid molecules or within a larger nucleic acid molecule. The target polynucleotide can be obtained from any source, and can comprise any number of different compositional components. For example, the target can be a nucleic acid (e.g. DNA or RNA). It will be appreciated that target polynucleotides can be cut or sheared prior to analysis, including the use of such procedures as mechanical force, sonication, restriction endonuclease cleavage, or other methods known in the art.

As used herein, the “polymerase chain reaction” or PCR comprises amplification of a nucleic acid consisting of an initial denaturation step which separates the strands of a double stranded nucleic acid sample, followed by repetition of (i) an annealing step, which allows amplification primers to anneal specifically to positions flanking a target sequence; (ii) an extension step which extends the primers in a 5′ to 3′ direction thereby forming an amplicon polynucleotide complementary to the target sequence, and (iii) a denaturation step which causes the separation of the amplicon from the target sequence (Mullis et al., EDS, The Polymerase Chain Reaction, BirkHauser, Boston, Mass. (1994)). Each of the above steps may be conducted at a different temperature, preferably using an automated thermocycler (Applied Biosystems LLC, a division of Life Technologies Corporation, Foster City, Calif.). If desired, RNA samples can be converted to DNA/RNA heteroduplexes or to duplex cDNA by methods known to one of skill in the art. PCR methods may also include reverse transcriptase-PCR and other reactions that follow principles of PCR.

As used herein “preparing” or “preparing a sample” or “processing” or processing a sample” refers to one or more of the following steps to achieve extraction and separation of a nucleic acid from a sample: (1) bacterial enrichment, (2) separation of bacterial cells from the sample, (3) cell lysis, and (4) nucleic acid extraction and/or purification (e.g., DNA extraction, total DNA extraction, genomic DNA extraction, RNA extraction). Embodiments of the nucleic acid extracted include, but are not limited to, DNA, RNA, mRNA and miRNA.

As used herein, “presence” refers to the existence (and therefore to the detection) of a reaction, a product of a method or a process (including but not limited to, an amplification product resulting from an amplification reaction), or to the “presence” and “detection” of an organism such as a pathogenic organism or a particular strain or species of an organism.

The term “primer” refers to a polynucleotide and analogs thereof that are capable of selectively hybridizing to a target nucleic acid or a “template,” a target region flanking sequence or to a corresponding primer-binding site of an amplification product; and allows detection of a double-stranded nucleic acid formed by hybridization or the synthesis of a sequence complementary to the corresponding polynucleotide template, flanking sequence or amplification product from the primer's 3′ end. Typically a primer can be between about 10 to 100 nucleotides in length and can provide a point of initiation for template-directed synthesis of a polynucleotide complementary to the template, which can take place, in the presence of appropriate enzyme(s), cofactors, substrates such as nucleotides and the like.

As used herein, the term “amplification primer” refers to an oligonucleotide, capable of annealing to an RNA or DNA region adjacent a target nucleic acid sequence, and serving as an initiation primer for nucleic acid synthesis under suitable conditions well known in the art. Typically, a PCR reaction employs a pair of amplification primers including an “upstream” or “forward” primer and a “downstream” or “reverse” primer, which delimit a region of the RNA or DNA to be amplified.

As used herein, the term “primer-binding site” refers to a region of a polynucleotide sequence, typically a sequence flanking a target region and/or an amplicon that can serve directly, or by virtue of its complement, as the template upon which a primer can anneal for any suitable primer extension reaction known in the art, for example, but not limited to, PCR. It will be appreciated by those of skill in the art that when two primer-binding sites are present on a single polynucleotide, the orientation of the two primer-binding sites is generally different. For example, one primer of a primer pair is complementary to and can hybridize with the first primer-binding site, while the corresponding primer of the primer pair is designed to hybridize with the complement of the second primer-binding site. Stated another way, in some embodiments the first primer-binding site can be in a sense orientation, and the second primer-binding site can be in an antisense orientation. A primer-binding site of an amplicon may, but need not comprise the same sequence as or at least some of the sequence of the target flanking sequence or its complement.

The terms “reporter probe” and “probe” are used interchangeably and refer to a detectable sequence of nucleotides or a detectable sequence of nucleotide analogs operable to specifically anneal with a corresponding amplicon, such as but not limited to, a target nucleic acid sequence and/or a PCR product and is further operable to be detected or identified. Reporter probes or probes may be detectable by a variety of methods, including but not limited to, detecting color, detecting radiation, fluorescence, luminescence, emitted wavelengths. In some embodiments, detecting a change in intensity, a change in radiation, a change in an emitted wavelength, a change in fluorescence, a change in luminescence, or a change in color or intensity of color may be used to identify and/or quantify a corresponding amplicon or a target polynucleotide. In one exemplary embodiment, by indirectly detecting an amplicon from a sample or processed sample, one can determine that a microorganism having a corresponding target sequence is present in a sample. Most reporter probes can be categorized based on their mode of action, for example but not limited to: nuclease probes, including without limitation TaqMan® probes; extension probes including without limitation scorpion primers, Lux™ primers, Amplifluors, and the like; and hybridization probes including without limitation molecular beacons, Eclipse probes, light-up probes, pairs of singly-labeled reporter probes, hybridization probe pairs, and the like. In certain embodiments, reporter probes may comprise an amide bond, an LNA, a universal base, and/or combinations thereof, and may include stem-loop and/or stem-less reporter probe configurations. Certain reporter probes may be singly-labeled, while other reporter probes are doubly-labeled. Dual probe systems that comprise FRET between adjacently hybridized probes are within the intended scope of the term reporter probe. In certain embodiments, a reporter probe may comprise a fluorescent reporter group and a quencher (including without limitation dark quenchers and fluorescent quenchers). Some non-limiting examples of reporter probes include TaqMan® probes; Scorpion probes (also referred to as scorpion primers); Lux™ primers; FRET primers; Eclipse probes; molecular beacons, including but not limited to FRET-based molecular beacons, multicolor molecular beacons, aptamer beacons, PNA beacons, and antibody beacons; labeled PNA clamps, labeled PNA openers, labeled LNA probes, and probes comprising nanocrystals, metallic nanoparticles and similar hybrid probes (see, e.g., Dubertret et al., Nature Biotech., 19:365-70, 2001; Zelphati et al., BioTechniques 28:304-15, 2000). In certain embodiments, reporter probes may further comprise minor groove binders including but not limited to TaqMan® MGB probes and TaqMan® MGB-NFQ probes (both from Applied Biosystems). In certain embodiments, reporter probe detection may comprise fluorescence polarization detection (see, e.g., Simeonov and Nikiforov, Nucl. Acids Res. 30:E91, 2002).

Those skilled in the art understand that as a target nucleic acid region (target sequence) is amplified by an amplification means, the complement of the primer-binding site is synthesized in the complementary amplicon or the complementary strand of the amplicon. Accordingly, it is to be understood that the complement of a primer-binding site is expressly included within the intended meaning of the term primer-binding site, as used herein.

As used herein, the term “genome” refers to the complete nucleic acid sequence, containing the entire genetic information, of a bacterium, a virus, a plasmid, a gamete, an individual, a population, a species, or a strain of a species.

As used herein, the term “pseudochromosome” refers to the concatenation, in their most likely order, of all available sequence contigs and scaffolds derived from sequencing of a bacterial genome, in which undefined gaps between contigs and scaffolds are represented by unidentified nucleobases.

As used herein, the term “genomic DNA” refers to the chromosomal DNA sequence of a gene or segment of a gene including the DNA sequence of non-coding as well as coding regions. Genomic DNA also refers to DNA isolated directly from cells, chromosomes or plasmid(s) within the genome of an organism, or cloned copies of all or part of such DNA.

As used herein the term “sample” refers to a starting material suspected of harboring a particular microorganism or group of microorganisms. A “contaminated sample” refers to a sample harboring a pathogenic microbe thereby comprising nucleic acid material from the pathogenic microbe. Examples of samples include, but are not limited to, food samples (including but not limited to samples from food intended for human or animal consumption such as processed foods, raw food material, produce (e.g., fruit and vegetables), legumes, meats (from livestock animals and/or game animals), fish, sea food, nuts, beverages, drinks, fermentation broths, and/or a selectively enriched food matrix comprising any of the above listed foods), water samples, environmental samples (e.g., soil samples, dirt samples, garbage samples, sewage samples, industrial effluent samples, air samples, or water samples from a variety of water bodies such as lakes, rivers, ponds etc.), air samples (from the environment or from a room or a building), forensic samples, agricultural samples, pharmaceutical samples, biopharmaceutical samples, samples from food processing and manufacturing surfaces, and/or biological samples. A “biological sample” refers to a sample obtained from eukaryotic or prokaryotic sources. Examples of eukaryotic sources include mammals, such as a human, a cow, a pig, a chicken, a turkey, a livestock animal, a fish, a crab, a crustacean, a rabbit, a game animal, and/or a member of the family Muridae (a murine animal such as rat or mouse). A biological sample may include blood, urine, feces, or other materials from a human or a livestock animal. Examples of prokaryotic sources include enterococci. A biological sample can be, for instance, in the form of a single cell, in the form of a tissue, or in the form of a fluid.

A sample may be tested directly, or may be prepared or processed in some manner prior to testing. For example, a sample may be processed to enrich any contaminating microbe and may be further processed to separate and/or lyse microbial cells contained therein. Lysed microbial cells from a sample may be additionally processed or prepares to separate, isolate and/or extract genetic material from the microbe for analysis to detect and/or identify the contaminating microbe. Analysis of a sample may include one or more molecular methods. For example, according to some exemplary embodiments of the present disclosure, a sample may be subject to nucleic acid amplification (for example by PCR) using appropriate oligonucleotide primers that are specific to one or more microbe nucleic acid sequences that the sample is suspected of being contaminated with. Amplification products may then be further subject to testing with specific probes (or reporter probes) to allow detection of microbial nucleic acid sequences that have been amplified from the sample. In some embodiments, if a microbial nucleic acid sequence is amplified from a sample, further analysis may be performed on the amplification product to further identify, quantify and analyze the detected microbe (determine parameters such as but not limited to the microbial strain, pathogenecity, quantity etc.).

Recitation of numerical ranges by endpoints in this specification include all numbers subsumed within that range (e.g., 1 to 5 includes 1, 1.5, 2, 2.75, 3, 3.80, 4, 5, etc.).

Various embodiments of the present teachings relate to compositions, methods and kits for identification of an E. coli O55:H7 microorganism. E. coli O55:H7 is known to cause human disease and hence is a pathogen that is a potential food contaminant, an environmental contaminant and may be a used as a biowarfare agent or a bioterrorism agent.

The present disclosure, in some embodiments discloses nucleotide sequences specific to E. coli O55:H7 and discloses detection assays designed using nucleotide sequences specific for this E. coli serotype. The specific and unique sequences were discovered by whole-genome sequencing of the bacterium E. coli O55:H7. The entire genome of a strain of E. coli O55:H7 is presented herein, providing the genomic information necessary to design highly specific E. coli O55:H7 assays. Embodiments relating to sequencing E. coli O55:H7 are described in the section entitled Examples.

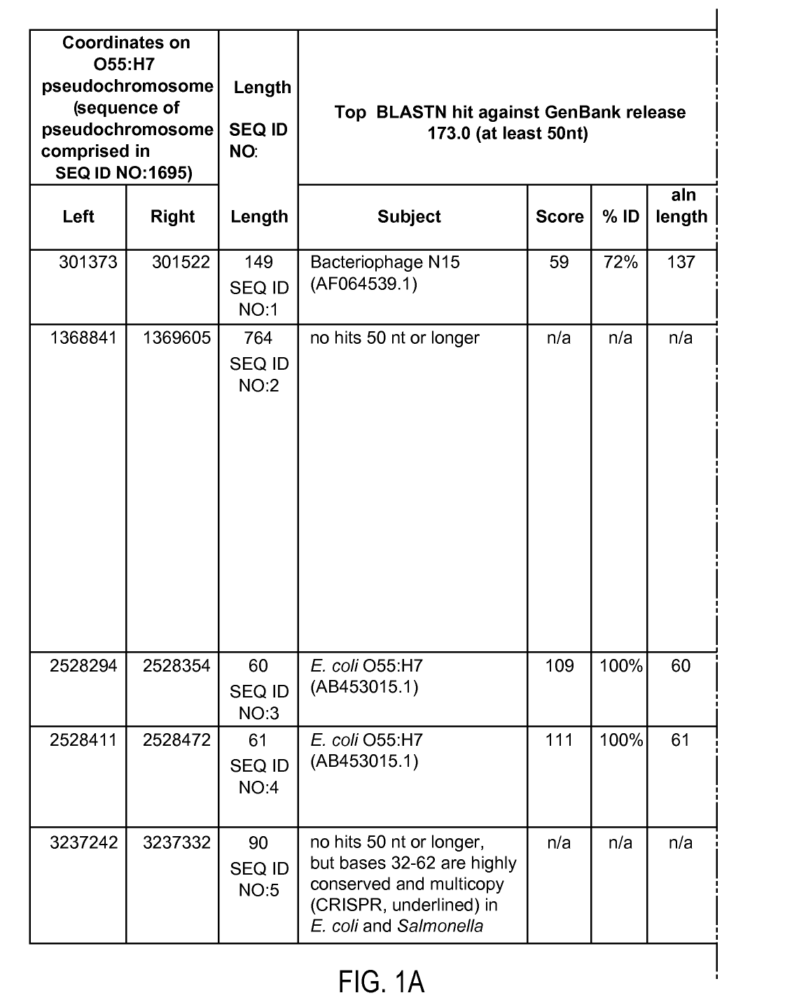

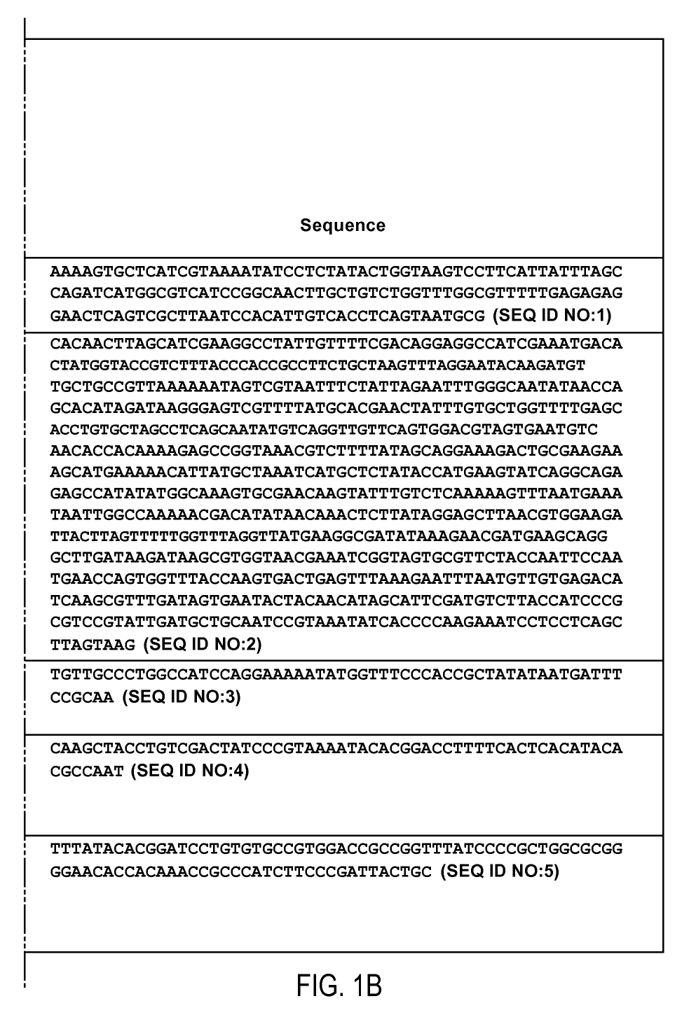

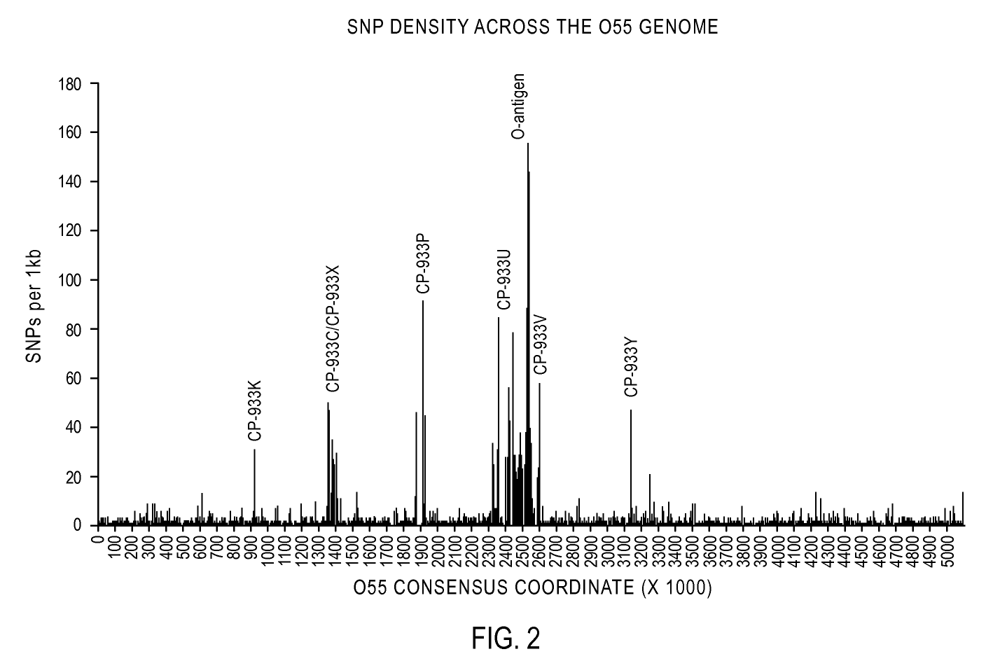

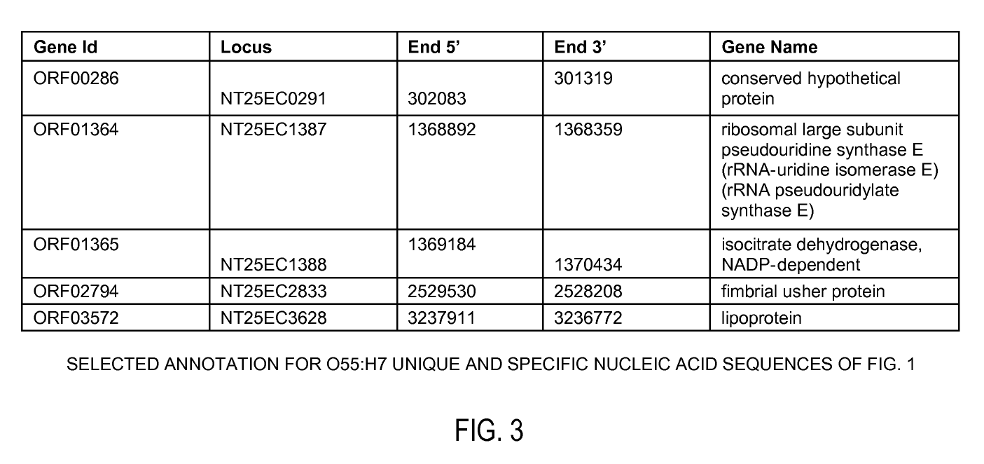

Various embodiments of the present teachings relate to compositions based on newly discovered genomic sequence regions specific and unique to E. coli O55:H7. The entire genomic sequence as sequences is provided in the concurrently filed sequence listing. Example compositions of the disclosure include isolated sequences described in FIG. 1 that are uniquely found in E. coli O55:H7 but not in other closely related E. coli strains. These include, in some exemplary embodiments, at least isolated nucleic acid sequences described herein as SEQ ID NO: 66, SEQ ID NO: 252, SEQ ID NO: 1113, SEQ ID NO: 1461, SEQ ID NO:1, SEQ ID NO: 2, SEQ ID NO: 3, SEQ ID NO: 4 and SEQ ID NO: 5, fragments thereof and complements thereof. Compositions of the disclosure also include sequences that are complements of, fragments of, and/or sequences comprising at least 90% nucleic acid sequence identity to the sequences set forth in FIG. 1 and/or described herein as SEQ ID NO: 66, SEQ ID NO: 252, SEQ ID NO: 1113, SEQ ID NO: 1461, SEQ ID NO:1, SEQ ID NO: 2, SEQ ID NO: 3, SEQ ID NO: 4 and SEQ ID NO: 5. Nucleic acid sequences corresponding to SEQ ID NO: 66, SEQ ID NO: 252, SEQ ID NO: 1113, SEQ ID NO: 1461, SEQ ID NO:1, SEQ ID NO: 2, SEQ ID NO: 3, SEQ ID NO: 4 and SEQ ID NO: 5 are provided in the Sequence Listing and also in Table 5.

In some embodiments, isolated nucleic acid sequences of the disclosure may comprise nucleic acid molecules comprising at least a 40 nucleotide sequence of SEQ ID NO:66, SEQ ID NO:252, SEQ ID NO:1113, SEQ ID NO:1461, SEQ ID NO:1, SEQ ID NO:2, SEQ ID NO:3, SEQ ID NO:4, SEQ ID NO:5; at least a 30 nucleotide sequence of SEQ ID NO:66, SEQ ID NO:252, SEQ ID NO:1113, SEQ ID NO:1461, SEQ ID NO:1, SEQ ID NO:2, SEQ ID NO:3, SEQ ID NO:4, SEQ ID NO:5; at least a 25 nucleotide sequence of SEQ ID NO:66, SEQ ID NO:252, SEQ ID NO:1113, SEQ ID NO:1461, SEQ ID NO:1, SEQ ID NO:2, SEQ ID NO:3, SEQ ID NO:4, SEQ ID NO:5; at least a 20 nucleotide sequence of SEQ ID NO:66, SEQ ID NO:252, SEQ ID NO:1113, SEQ ID NO:1461, SEQ ID NO:1, SEQ ID NO:2, SEQ ID NO:3, SEQ ID NO:4, SEQ ID NO:5; at least a 15 nucleotide sequence of SEQ ID NO:66, SEQ ID NO:252, SEQ ID NO:1113, SEQ ID NO:1461, SEQ ID NO:1, SEQ ID NO:2, SEQ ID NO:3, SEQ ID NO:4, SEQ ID NO:5; at least a 10 nucleotide sequence of SEQ ID NO:66, SEQ ID NO:252, SEQ ID NO:1113, SEQ ID NO:1461, SEQ ID NO:1, SEQ ID NO:2, SEQ ID NO:3, SEQ ID NO:4, SEQ ID NO:5; any intermediate number of contiguous sequences from at least about 10 nucleotides of sequence to at least about 25 nucleotides of sequence of SEQ ID NO:66, SEQ ID NO:252, SEQ ID NO:1113, SEQ ID NO:1461, SEQ ID NO:1, SEQ ID NO:2, SEQ ID NO:3, SEQ ID NO:4, SEQ ID NO:5 and sequences having 90% identity to the foregoing sequences.

The present disclosure also provides in some embodiments compositions comprising primer and/or probe sequences that may be used for detection, identification, quantitation and/or differential detection of an E. coli O55:H7 organism. Probes and/or primers generally comprise, but are not limited to, oligonucleotide sequence having from about 10 to about 40 nucleotides. Exemplary probe and/or primer compositions of the disclosure include, but are not limited to, an isolated nucleic acid molecules having nucleic acid sequences comprised in SEQ ID NOs: 6-32, and/or nucleic acid sequences having at least 90% sequence identity to nucleic acid sequences comprised in SEQ ID NOs: 6-32, and/or fragments thereof, and/or oligonucleotide sequences having from at least 10 contiguous nucleotides of SEQ ID NOs: 6-32, oligonucleotide sequences having from at least 15 contiguous nucleotides of SEQ ID NOs: 6-32, oligonucleotide sequences having from at least 20 contiguous nucleotides of SEQ ID NOs: 6-32, and/or complementary sequences thereof. The sequences described in the sentence above are referred to collectively as “sequences comprising or derived from SEQ ID NOS: 6-32.” Nucleic acids corresponding to SEQ ID NOS: 6-32 are described in the Sequence Listing as well as in Table 5.

In some embodiments, exemplary probe and/or primer sequences set forth above comprising or derived from SEQ ID NOs: 6-32 may also comprise a label. A label may include, but is not limited to, a dye, a radioactive isotope, a fluorescent label, a bioluminescent label, a chemiluminescent label, an enzyme. A dye in some embodiments may be a fluorescein dye, a rhodamine dye, a cyanine dye, such as but not limited to FAM™ dye, and/or a VIC® dye.

In some embodiments, probes and/or primers of the disclosure for detection, identification, quantitation and/or differential detection methods and/or steps that are described in sections below. These methods may comprise embodiments such as hybridization that utilize one or more probe sequences of the disclosure, such as, but not limited, to sequences comprising or derived from SEQ ID NOS: 6-32; embodiments such as amplification (e.g., PCR) utilizing at least one primer pair of the disclosure, such as, but not limited, to sequences comprising or derived from SEQ ID NOS: 6-32; embodiments such as multiplex amplification using multiple primer pairs, such as, but not limited, to sequences comprising or derived from SEQ ID NOS: 6-32; embodiments such as quantitative detection (e.g., by real-time PCR) of amplified DNA using at least one probe and at least one primer pair.

Embodiments of the disclosure also relate to designing additional probe and/or primer sequences based on unique regions specific to E. coli O55:H7 described herein. Several programs and algorithms may be used to design primers and/or probes based on the nucleotide sequences specific to E. coli O55:H7 that are disclosed in the present specification. Probe or primer compositions of the disclosure may be synthesized or isolated by methods known in the art in light of the teachings of the present disclosure and the sequences provided herein. In some embodiments, a probe or a primer may comprise a sequence having as few as 10 nucleic acids, at least 15, at least 20 and at least about 25 nucleotides in length to at least about 40 nucleotides in length may be used.

Recombinant constructs comprising a probe and/or a primer sequence of the disclosure may comprise, but are not limited to, a recombinant construct comprising a sequences comprising or derived from SEQ ID NOS: 6-32.

Some embodiments describe methods for detection and identification of one or more unique sequences in a target nucleic acid extracted from or present in a sample suspected of containing an E. coli to identify the microorganism as E. coli O55:H7. E. coli O55:H7 specific and unique sequences may be identified alone or in any combination in order to identify or determine the presence of E. coli O55:H7. Exemplary sequences that are unique to E. coli O55:H7 are set forth in FIG. 1 and/or described herein as SEQ ID NO: 66, SEQ ID NO: 252, SEQ ID NO: 1113, SEQ ID NO: 1461, SEQ ID NO:1, SEQ ID NO: 2, SEQ ID NO: 3, SEQ ID NO: 4 and SEQ ID NO: 5.

Methods of the disclosure may be used for diagnostic detection and testing methods (such as for food safety testing) and are useful to prevent and protect against E. coli O55:H7 based human/animal infections.

In some embodiments, methods for detection of E. coli O55:H7 may comprise detecting in a sample at least one (or more) of a nucleic acid sequence selected from the group consisting of SEQ ID NO: 66, SEQ ID NO: 252, SEQ ID NO: 1113, SEQ ID NO: 1461, SEQ ID. NO: 1, SEQ ID NO:2, SEQ ID NO:3, SEQ ID NO:4, SEQ ID NO:5, fragments thereof, and complements thereof, wherein detection of one of the at least one nucleic acid sequences identifies E. coli O55:H7. Methods may also employ sequences that have at least 90% nucleic acid sequence identity to these sequences.

An exemplary testing method may comprise: preparing a sample which may comprise: a) processing a sample to extract any genetic material contained in the sample and to render the genetic material amenable to detection steps (e.g., isolating nucleic acid from a sample); b) providing a composition of the disclosure comprising at least one isolated nucleotide sequence of an E. coli O55:H7-specific nucleotide sequence (such as but not limited to at least one nucleic acid sequence having the sequence of SEQ ID NO: 66, SEQ ID NO: 252, SEQ ID NO: 1113, SEQ ID NO: 1461, SEQ ID NO:1, SEQ ID NO: 2, SEQ ID NO: 3, SEQ ID NO: 4, SEQ ID NO: 5, a fragment of the foregoing nucleic acids (also referred to as fragments thereof), a nucleic acid having from at least 10 to at least 25 nucleotides of contiguous sequences of the foregoing sequences, complements thereof and/or sequences comprising at least 90% nucleic acid sequence identity thereof); c) contacting the at least one E. coli O55:H7-specific isolated nucleotide sequence with the sample (processed sample); and d) detecting hybridization of the at least one E. coli O55:H7-specific nucleotide sequence to a complementary nucleotide sequence in the sample. Detecting one or more nucleotide sequences that are unique to E. coli O55:H7 are indicative that the test sample contains E. coli O55:H7. Embodiments of the disclosure also describe quantitative assays by which one of skill in the art, in light of this disclosure, may quantify the amount of E. coli O55:H7 in the sample.

In some embodiments, a nucleic acid may be isolated from a sample prior to practicing a method of the disclosure by isolating nucleic acids by methods known in the art to isolate nucleic acids from samples. Samples of various kinds as described in sections above may be amenable to the methods. In some embodiments, methods of the disclosure may comprise testing a food sample for contamination by E. coli O55:H7 and may comprise isolating nucleic acid from a food sample having a selectively enriched food matrix.

Detecting the at least one nucleic acid sequence from a sample may be performed by one or more technologies, such as, but not limited to, nucleic acid amplification, hybridization, mass spectrometry, nanostring, microfluidics, chemiluminescence, enzyme technologies and combinations thereof. Some of these technologies are described in later sections of the specification.

In one embodiment, a method of the disclosure for specifically detecting E. coli O55:H7 may comprise identifying at least a first unique region specific to E. coli O55:H7 referred to as a “first target nucleic acid sequence” for detection, obtaining or designing one or more primer pairs (polynucleotides) each primer pair comprising a “first primer” operable to hybridize to a first sequence within the first target nucleic acid sequence and at least a “second primer” operable to hybridize to a second sequence within the first target nucleic acid sequence; hybridizing at least a first pair to the first target nucleic acid sequence; amplifying the first target nucleic acid sequence to form a first amplified target nucleic acid sequence product; and detecting the at least first amplified target nucleic acid sequence product, wherein detection of the at least first amplified target nucleic acid sequence product is indicative of the presence of E. coli O55:H7. In some embodiments, the method is also indicative of the absence of E. coli O157:H7 in the sample and/or the absence of non-E. coli O55:H7 bacteria.

In some embodiments, a method as described above may further comprise: identifying at least a second target nucleic acid sequence specific to E. coli O55:H7; hybridizing a second pair of polynucleotide primers to the second target nucleic acid sequence; amplifying the second target nucleic acid sequence to form a second amplified target nucleic acid sequence product; and detecting the second amplified target nucleic acid sequence product, wherein detection of the second amplified target nucleic acid sequence product is indicative of the presence of E. coli O55:H7. In some embodiments, the detection of the first and second amplified target nucleic acid sequence product indicates the presence of E. coli O55:H7. Multiple targets nucleic acids may be amplifies and identified to increase the specificity of the assay if desired.

In some embodiments, the first target nucleic acid sequence specific to E. coli O55:H7 and the second target nucleic acid sequence specific to E. coli O55:H7 may comprise one or more sequences such as but not limited to: SEQ ID NO: 66, SEQ ID NO: 252, SEQ ID NO: 1113, SEQ ID NO: 1461, SEQ ID NO:1, SEQ ID NO: 2, SEQ ID NO: 3, SEQ ID NO: 4, SEQ ID NO: 5, fragments thereof, at least 25 nucleotide sequences thereof, complements thereof and sequences comprising at least 90% nucleic acid sequence identity thereof.

The first primer pair and the second primer pair of the methods, in some embodiments, may be one or more of: SEQ ID NO: 6, SEQ ID NO:7, SEQ ID NO:8, SEQ ID NO:9, SEQ ID NO:10, SEQ ID NO: 11, SEQ ID NO:12, SEQ ID NO:13, SEQ ID NO:14, SEQ ID NO:15, SEQ ID NO:16, SEQ ID NO: 17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, fragments thereof, at least 10 contiguous nucleotide sequences thereof complements thereof, and labeled derivatives thereof.

In some embodiments, detection of an amplified target nucleic acid sequence product (such as a first amplified target nucleic acid sequence product and/or a second amplified target nucleic acid sequence product) as set forth in the embodiment methods described above may comprise use of a probe. Exemplary probes may comprise but are not limited to one or more sequences such as SEQ ID NO: 6, SEQ ID NO:7, SEQ ID NO:8, SEQ ID NO:9, SEQ ID NO:10, SEQ ID NO: 11, SEQ ID NO:12, SEQ ID NO:13, SEQ ID NO:14, SEQ ID NO:15, SEQ ID NO:16, SEQ ID NO: 17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, fragments thereof, at least 10 contiguous nucleotide sequences thereof complements thereof, and labeled derivatives thereof.

Labeled probes and/or primers are helpful in detection and quantitation methods. Label for primers and probes may comprise at least one of the following: a dye, a radioactive isotope, a chemiluminescent label, a fluorescent label, a bioluminescent label, and an enzyme. Dye's may comprise a fluorescein dye, a rhodamine dye, and/or a cyanine dye. Some probes and primers may be dually labeled. Non-limiting examples of nucleic acid dyes include ethidium bromide, DAPI, Hoechst derivatives including without limitation Hoechst 33258 and Hoechst 33342, intercalators comprising a lanthanide chelate (for example but not limited to a nalthalene diimide derivative carrying two fluorescent tetradentate β-diketone-Eu³⁺ chelates (NDI-(BHHCT-Eu³⁺)₂), (See, e.g., Nojima et al., Nucl. Acids Res. Supplement No. 1, 105-06 (2001)), ethidium bromide, and certain unsymmetrical cyanine dyes such as SYBR® Green, PicoGreen®, and BOXTO dyes. SYBR Green dye is an “intercalating dye” which, as used herein, refers to a fluorescent molecule that is specific for a double-stranded polynucleotide or that at least shows a substantially greater fluorescent enhancement when associated with a double-stranded polynucleotide than with a single-stranded polynucleotide. Typically nucleic acid dye molecules associate with double-stranded segments of polynucleotides by intercalating between the base pairs of the double-stranded segment, by binding in the major or minor grooves of the double-stranded segment, or both.

Various embodiments of the present teachings relate to a multi-primer assay for detecting E. coli O55:H7 in a sample. Methods of the disclosure, in some embodiments, comprise amplification methods that yield one or more amplification products. In some embodiments an amplification product may be detected by a real-time assay. A real-time assay may be, but is not limited to a SYBR® Green dye assay or a TaqMan® assay.

In embodiments of methods where more than one (e.g., two) amplification products may be formed, detection of a first amplification product may entail the use of a first probe and detection of a second amplification product may entail the use of a second probe. In such embodiments, a first probe may have a first label and a second probe may comprise a second label. In one example embodiment, a first probe may be labeled with a FAM™ dye and a second probe may be labeled with VIC® dye. In some embodiments, hybridizing and amplifying with a first pair of polynucleotide primers may be carried out in a first vessel and hybridizing and amplifying with a second pair of polynucleotide primers may be carried in a second vessel. In some embodiments, hybridizing and amplifying with a first pair of polynucleotide primers and hybridizing and amplifying with a second pair of polynucleotide primers may be carried out in a single vessel. In some embodiments, detection of amplified products may be by a real-time assay such as a SYBR® Green dye assay or a TaqMan® assay.

In some embodiments, the present disclosure describes methods based on utilizing whole-genome sequencing of a bacterium(s) and/or bacterial strain(s) of interest (e.g., E. Coli O55:H7) and comparison to other known bacterial organisms (e.g., E. Coli O157:H7) to identifying the bacterium of interest.

For example, some embodiments of the disclosure describe assays to distinguish E. coli O55:H7 from E. coli O157:H7. E. coli O157:H7 is a known pathogen that is highly similar at the nucleotide level to the E. coli O55:H7 serotype. Tests to detect E. coli O157:H7 often cross detect E. coli O55:H7, thereby picking up false positives. The present disclosure provides nucleotide sequence information that may be used to design specific tests for the distinct detection of E. coli O157:H7 that does not cross-detect E. coli O55:H7. For example, in some embodiments, using the genome sequence of E. coli O55:H7 as described herein and the genomic sequence of E. coli O157:H7, primers and probes may be designed that detect sequences unique to E. coli O157:H7 that are not present in E. coli O55:H7.

In other embodiments, a specific testing method may comprise: testing a sample that has been detected to be positive for E. coli O157:H7 comprising: a) providing an isolated nucleotide sequence of an E. coli O55:H7-specific nucleotide sequence (such as but not limited to at least one nucleic acid sequence having the sequence of SEQ ID NO: 66, SEQ ID NO: 252, SEQ ID NO: 1113, SEQ ID NO: 1461, SEQ ID NO:1, SEQ ID NO: 2, SEQ ID NO: 3, SEQ ID NO: 4, SEQ ID NO: 5, a fragment of the foregoing nucleic acids (also referred to as fragments thereof), a nucleic acid having at least 25 nucleotides of contiguous sequences of the foregoing sequences, complements thereof and/or sequences comprising at least 90% nucleic acid sequence identity thereof; b) contacting the at least one E. coli O55:H7-specific isolated nucleotide sequence with the sample; and c) detecting hybridization of the at least one E. coli O55:H7-specific nucleotide sequence to a complementary nucleotide sequence in the sample. Detecting one or more nucleotide sequences that are unique to E. coli O55:H7 are indicative that the test sample contains E. coli O55:H7. Several exemplary detecting methods that may be used have been described in sections above. Embodiments of the disclosure also describe quantitative assays by which one of skill in the art, in light of this disclosure, may quantify the amount of E. coli O55:H7 in the sample. This may be compared to the quantity of E. coli O157:H7 detected in the sample to determine whether the sample is devoid of E. coli O157:H7 or is contaminated with a combination of E. coli O157:H7 and E. coli O55:H7.

In some embodiments, methods for distinguishing a bacteria from an E. coli O55:H7 are described and may comprise analyzing the genome of the bacteria for the presence of a sequence selected from the group consisting of SEQ ID NO:1, SEQ ID NO:66, SEQ ID NO:2, SEQ ID NO:252, SEQ ID NO:3, SEQ ID NO:4, SEQ ID NO:1113, SEQ ID NO:5 and SEQ ID NO:1461, fragments thereof, at least 25 nucleotide sequences thereof and sequences comprising at least 90% nucleic acid sequence identity thereof. Such methods may be used to distinguish the presence of E. coli O55:H7 from a bacterium of several species. For example, methods of the disclosure may be used to distinguish the presence of E. coli O55:H7 from other E. coli bacteria such as an E. coli O26:H11. Methods of the disclosure may also be used to distinguish the presence of E. coli O55:H7 from a bacteria of a Salmonella sp. and/or Shigella spp. In some embodiments, the Shigella spp. may be Shigella dysenteriae, Shigella flexneri, Shigella boydii and Shigella sonnei. In some embodiments, Shigella dysentaeria may be a strain selected from the group consisting of strain 1012, strain M131649 and strain Sd197. In some embodiments, the Shigella flexneri may be a strain selected from the group consisting of strain 2457T, strain 301 and strain 8401. In some embodiments, Shigella boydii may be a strain selected from the group consisting of strain BS512 and strain Sb227. In some embodiments, the Shigella sonnei may be a strain selected from the group consisting of strain 53G and strain Ss046.

Methods of the disclosure may further comprise preparing a test sample for amplification prior to hybridizing and/or amplification and may include steps such as but not limited to (1) bacterial enrichment, (2) separation of bacterial cells from other components of the sample, (3) lysis of bacterial cells, and (4) nucleic acid extraction.

In various embodiments, a variety of methods for amplifying nucleic acid sequences may be employed. Amplification may be mediated by polymerase chain reaction, having at least a first pair of polynucleotide primers and in some embodiments at least a second pair of polynucleotide primers. Amplification methods include, but are not limited to, polymerase chain reaction (PCR), RT-PCR, asynchronous PCR (A-PCR), and asymmetric PCR (AM-PCR), strand displacement amplification (SDA), multiple displacement amplification (MDA), nucleic acid strand-based amplification (NASBA), and/or rolling circle amplification (RCA), transcription-mediated amplification (TMA). (See, e.g., PCR Technology: Principles and Applications for DNA Amplification (ed. H. A. Erlich, Freeman Press, NY, N.Y., 1992); PCR Protocols: A Guide to Methods and Applications (eds. Innis, et al., Academic Press, San Diego, Calif., 1990); Mattila et al., Nucleic Acids Res. 19, 4967 (1991); Eckert et al., PCR Methods and Applications 1, 17 (1991); PCR (eds. McPherson et al., IRL Press, Oxford); and U.S. Pat. Nos. 4,683,202, 4,683,195, 4,800,159 4,965,188 and 5,333,675 each of which is incorporated herein by reference in their entirety).

Nucleic acid amplification techniques are traditionally classified according to the temperature requirements of the amplification process. Isothermal amplifications are conducted at a constant temperature, in contrast to amplifications that require cycling between high and low temperatures. Examples of isothermal amplification techniques are: Strand Displacement Amplification (SDA; Walker et al., 1992, Proc. Natl. Acad. Sci. USA 89:392 396; Walker et al., 1992, Nuc. Acids. Res. 20:1691 1696; and EP 0 497 272, all of which are incorporated herein by reference), self-sustained sequence replication (3SR; Guatelli et al., 1990, Proc. Natl. Acad. Sci. USA 87:1874 1878), the Qβ replicase system (Lizardi et al., 1988, BioTechnology 6:1197 1202), and the techniques disclosed in WO 90/10064 and WO 91/03573.

Examples of techniques that require temperature cycling are: polymerase chain reaction (PCR; Saiki et al., 1985, Science 230:1350 1354), ligase chain reaction (LCR; Wu et al., 1989, Genomics 4:560 569; Barringer et al., 1990, Gene 89:117 122; Barany, 1991, Proc. Natl. Acad. Sci. USA 88:189 193), transcription-based amplification (Kwoh et al., 1989, Proc. Natl. Acad. Sci. USA 86:1173 1177) and restriction amplification (U.S. Pat. No. 5,102,784), and self-sustained sequence replication (Guatelli et al., Proc. Nat. Acad. Sci. USA, 87, 1874 (1990)) and nucleic acid based sequence amplification (NABSA). (See, U.S. Pat. Nos. 5,409,818, 5,554517 and 6,063,603). The latter two amplification methods include isothermal reactions based on isothermal transcription, which produce both single-stranded RNA (ssRNA) and double-stranded DNA (dsDNA) as the amplification products in a ratio of about 30 or 100 to 1, respectively.

Other exemplary techniques include Nucleic Acid Sequence-Based Amplification (“NASBA”; see U.S. Pat. No. 5,130,238), and Rolling Circle Amplification (see Lizardi et al., Nat Genet. 19:225 232 (1998)). Amplification primers comprising nucleic acid sequences unique to E. coli O55:H7 and/or designed based on these unique E. coli O55:H7 sequences of the present disclosure may be used to carry out, for example, but not limited to, PCR, SDA or tSDA.

PCR is an extremely powerful technique for amplifying specific polynucleotide sequences, including genomic DNA, single-stranded cDNA, and mRNA among others. Various methods of conducting PCR amplification and primer design and construction for PCR amplification using sequences disclosed in this specification are described in the present disclosure. Generally, in PCR a double-stranded DNA to be amplified is denatured by heating the sample. New DNA synthesis is then primed by hybridizing primers to one or more target sequence(s) in the presence of DNA polymerase and excess dNTPs. In subsequent cycles, the primers hybridize to the newly synthesized DNA to produce discreet products comprising the primer sequences at either end. These amplified products accumulate exponentially with each successive round of amplification. The DNA polymerase used in PCR is often a thermostable polymerase. This allows the enzyme to continue functioning after repeated cycles of heating necessary to denature the double-stranded DNA for allowing primer annealing. Polymerases that are useful for PCR include, but are not limited to, Taq DNA polymerase, Tth DNA polymerase, Tfl DNA polymerase, Tma DNA polymerase, Tli DNA polymerase, and Pfu DNA polymerase. There are many commercially available modified forms of these enzymes including: AmpliTaq® and AmpliTaq Gold® both available from Applied Biosystems. Many are available with or without a 3′ to 5′ proofreading exonuclease activity. See, for example, Vent® and Vent®. (exo-) available from New England Biolabs.

Amplified products may be detected using probes or labeled primers. Since primers are incorporated into the ends of an amplicon, in some embodiments, labeled probes that are complementary to the primer sequences may be used. Alternatively labeled probes may be used for detection. Several other methods for the detection of an amplified product (e.g., PCR amplification product) include, but are not limited to, gel electrophoresis, capillary electrophoresis, and are known to one of skill in the art and may be applicable in light of the teachings of the present disclosure.

The disclosure also describes kits for the detection of E. coli O55:H7. A kit of the disclosure may comprise at least one pair of amplification primers (e.g., PCR primers) that may be designed or derived from nucleic acid sequences of SEQ ID NO:1, SEQ ID NO: 2, SEQ ID NO: 3, SEQ ID NO: 4, SEQ ID NO: 5, SEQ ID NO: 66, SEQ ID NO: 252, SEQ ID NO: 1113, SEQ ID NO: 1461, fragments thereof, complementary sequences thereof, sequences comprising at least 90% nucleic acid sequence identity thereof and complementary sequences comprising at least 90% nucleic acid sequence identity thereof. In some embodiments, the primers of a kit may be labeled. A kit comprising two (or more) pairs of primers may have primer pairs labeled with at least two (or more) different labels that may be detectable separately. A kit may further comprise at least one probe designed and/or derived from nucleic acid sequences comprising SEQ ID NO:1, SEQ ID NO: 2, SEQ ID NO: 3, SEQ ID NO: 4, SEQ ID NO: 5, SEQ ID NO: 66, SEQ ID NO: 252, SEQ ID NO: 1113, SEQ ID NO: 1461, fragments thereof, complementary sequences thereof, sequences comprising at least 90% nucleic acid sequence identity thereof and complementary sequences comprising at least 90% nucleic acid sequence identity thereof. Probes comprised in kits of the disclosure may be labeled. If a kit comprises multiple probes each probe may be labeled with a different label to allow detection of different products that may be the target of each different probe.

In some embodiments, a kit for the detection of E. coli O55:H7 may comprise: at least one pair of amplification primers (e.g., PCR primers) and/or at least one probe designed and/or derived from nucleic acid sequences comprising SEQ ID NO: 6, SEQ ID NO:7, SEQ ID NO:8, SEQ ID NO:9, SEQ ID NO:10, SEQ ID NO: 11, SEQ ID NO:12, SEQ ID NO:13, SEQ ID NO:14, SEQ ID NO:15, SEQ ID NO:16, SEQ ID NO: 17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, fragments comprising at least 10 contiguous nucleotide sequences thereof and complements thereof. In some embodiments, kit primers may be labeled. A kit comprising multiple pairs of primers may have primer pairs each labeled with different labels that may be detectable separately. Probes comprised in kits of the disclosure may be labeled. If a kit comprises multiple probes each probe may be labeled with a different label to allow detection of different products that may be the target of each different probe.

A kit of the disclosure may further comprise one or more components such as but not limited to: at least one enzyme, dNTPs, at least one buffer, at least one salt, at least one control nucleic acid sample, loading solution for preparation of the amplified material for electrophoresis, genomic DNA as a template control, a size marker to insure that materials migrate as anticipated in a separation medium, and an instruction protocol and manual to educate a user and limit error in use. It is within the scope of these teachings to provide test kits for use in manual applications or test kits for use with automated sample preparation, reaction set-up, detectors or analyzers. In some embodiments, a kit amplification product may be further analyzed by methods such as but not limited to electrophoresis, hybridization, mass spectrometry, nanostring, microfluidics, chemiluminescence and/or enzyme technologies.

Components of kits may be individually and in various combinations comprised in one or a plurality of suitable container means.

While the principles of inventions disclosed herein have been described in connection with specific embodiments, it should be understood clearly that these descriptions are made only by way of example and are not intended to limit the scope of inventions described herein. The present disclosure is for the purposes of illustration and description. It is not intended to be exhaustive or to limit disclosed embodiments to the precise forms as described. In light of this disclosure, many modifications and variations will be apparent to a practitioner skilled in the art. What is disclosed was chosen and described in order to best explain the principles and practical application of the disclosed embodiments of the art described, thereby enabling others skilled in the art to understand various embodiments and various modifications that are suited to contemplated uses. It is intended that the scope of what is disclosed be defined by the following claims and their equivalence.

### EXAMPLES

Some embodiments of the present disclosure may be understood in connection with the following examples. However, one skilled in the art will readily appreciate the specific materials, compositions, and results described are merely illustrative of the disclosure, and are not intended to, nor should be construed to, limit the scope disclosure and its various embodiments.

### Example I

### Strain Selection

One E. coli O55:H7 (PE704) bacterium strain was selected based on its close phylogenetic relationship with the E. coli O157:H7 strain. The E. coli O157:H7 (EDL933) strain sequence was used as a reference due to its availability as a finished genome in the public databases. Genomic DNA was isolated from a fresh bacterial lawn of E. coli O55:H7 strain PE704 using the DNeasy Blood and Tissue Kit (Qiagen, Valencia, Calif.) according to the manufacturer's directions.

### Example II

### SOLiD™ Sequencing

Mate-pair libraries were constructed from the isolated E. coli O55:H7 PE704 strain genomic DNA. Sequencing was carried out to 2×25 base pairs using SOLiD™ V1 chemistry (Applied Biosystems) according to the manufacturer's instructions.

### Example III

### Sequencing of E. coli O55:H7 O55:H7 Genome

In some embodiments, the genomic sequence of E. coli O55:H7 has been sequenced and specific and unique regions identified. The source of the E. coli O55:H7 nucleic acid used for sequencing is the strain PE704 (Applied Biosystems).

Genomic DNA was isolated from a fresh bacterial lawn of strain PE704 using the DNeasy Blood and Tissue Kit (Qiagen, Valencia, Calif.) according to the manufacturer's directions (Example I). The isolated genomic DNA was used to construct mate-pair libraries, which were sequenced to 2×25 base pairs using SOLiD™ V1 chemistry (Applied Biosystems), according to the manufacturer's instructions (Example II).

The complete genomic sequence of E. coli O55:H7, strain PE704 was sequenced using the SOLiD™ instrument platform using 25 nucleotide mate-paired reads. Mate-paired SOLiD reads from the E. coli O55:H7 PE704 genome were mapped against the E. coli O157:H7 EDL933 reference genome (Refseq Acc. NC_{—}002655.2) and from these, a consensus E. coli O55:H7 genomic sequence was derived. The consensus E. coli O55:H7 genomic sequence derived as set forth above is also referred to in this application as E. coli O55:H7 “pseudochromosome,” and its nucleic acid sequence is described in SEQ ID NO:1695, presented in concurrently filed Sequence Listing and described in Example IV below. The consensus sequence contained a number of gaps where sequence was not present in E. coli O55:H7 genome relative to E. coli O157:H7 genome. By breaking the consensus sequence at regions where read coverage dropped to zero, the E. coli O55:H7 genomic sequence assembly was reduced into contigs for which sequence was known, separated by gaps, in which sequence was unknown. Contig nucleic acid sequences are described in SEQ ID NOS: 33-1694, presented in concurrently filed Sequence Listing.

In order to close some of the gaps, long-range PCR was attempted for a number of these regions, and when successful, the resulting amplicons were sequenced using primer walking and Sanger sequencing methods. Some of these sequences represented E. coli O55:H7 insertions relative to the E. coli O157:H7 EDL933 genome. The ungapped SOLiD™ consensus contigs and Sanger sequence reads were assembled using GAP4 (R. Staden, D. P. Judge and J. K. Bonfield. Managing Sequencing Projects in the GAP4 Environment. Introduction to Bioinformatics. A Theoretical and Practical Approach. Eds. Stephen A. Krawetz and David D. Womble. Human Press Inc., Totawa, N.J. 07512 (2003)), yielding a total of 1,662 contigs (described in SEQ ID NOS: 33 through 1694, presented in concurrently filed Sequence Listing). Some of the E. coli O55:H7 genomic regions with a large number of clustered sequence gaps, primarily corresponding to some prophages in the E. coli O157:H7 EDL933 reference genome, were not able to be spanned by long-range PCR. The assembled sequence formed a pseudochromosome for E. coli O55:H7 (described in SEQ ID NO: 1695, see concurrently filed Sequence Listing) in which contigs were arranged in the most likely order and stitched together with intervening ambiguous spacers, indicated by one or more ‘N’ characters, which represent the undetermined bases in the inter-contig gaps.

The clustered sequence gaps are between each of SEQ ID NOS: 66 through 100, representing 34 gaps; SEQ ID NOS: 109 through 118, representing 9 gaps; SEQ ID NOS: 256 through 325, representing 69 gaps; SEQ ID NOS: 365 through 433, representing 68 gaps; SEQ ID NOS: 463 through 521, representing 58 gaps; SEQ ID NOS: 526 through 589, representing 63 gaps; SEQ ID NOS: 602 through 652, representing 50 gaps; SEQ ID NOS: 654 through 747, representing 93 gaps; SEQ ID NOS: 749 through 844, representing 95 gaps; SEQ ID NOS: 904 through 973, representing 69 gaps; SEQ ID NOS: 975 through 1042, representing 67 gaps; SEQ ID NOS: 1043 through 1050, representing 7 gaps; SEQ ID NOS: 1114 through 1120, representing 6 gaps; SEQ ID NOS: 1123 through 1320, representing 197 gaps; SEQ ID NOS: 1375 through 1385, representing 10 gaps; SEQ ID NOS: 1388 through 1447, representing 59 gaps. (See concurrently filed Sequence Listing for sequence information/description).

The consensus sequence (SEQ ID NO:1695, see concurrently filed Sequence Listing) was submitted to the JCVI Annotation Service (jcvi.org/cms/research/projects/annotation-service/overview/) for automated gene finding and annotation. The results, a listing of the open reading frames (ORFs) matched to putative functions were derived. A detailed listing of ORFs are described in one or more provisional applications, to which the present application claims priority to, and the specifications of which, are incorporated herein by reference in their entirety. FIG. 3 describes a selected annotation of ORFs for E. coli O55:H7 unique and/or specific nucleic acid sequences that are described in FIG. 1. An encoded protein from each ORF can be determined by converting the DNA sequence in an ORF to the corresponding amino acid sequence using the genetic code by methods known to one of skill in the art in light of the sequences and other teachings of the present disclosure.

The E. coli O55:H7 genome reads covered 91% of the E. coli O157:H7 EDL933 genome at an average depth of 20. The frequency of single nucleotide polymorphisms (SNPs) in the O55:H7 versus O157:H7 was 0.28%, confirming that the O55:H7 and O157:H7 serotypes are very closely related.

SNPs were detected by aligning the E. coli O55:H7 consensus sequence to the E. coli O157:H7 EDL933 sequence using the MUMmer suite (Kurtz, S. et al., (2004) Genome Biol. 5 R12.). SNPs in indel regions and in the artificial spacer sequences used to separate the contigs were omitted. Table 1 indicates the number of SNPs identified in the E. coli O55:H7 genome and Table 2 identifies the few regions having greater than half (54.3%) of the total SNPs, comprising 165 Kb (about 3% of the genome). Coordinates refer to the E. coli O55:H7 consensus genome (i.e, to nucleic acid residues as numbered in SEQ ID NO:1695, see concurrently filed Sequence Listing). The plot of SNP density in 1 Kb windows across the O55:H7 genome is shown in FIG. 2.

TABLE 1

| | |
|---|---|
| Non-coding SNPS | 985 |
| Synonymous coding SNPs | 3,534 |
| Non-synonymous coding SNPs | 2,445 |
| Total SNPS | 6,964 |

TABLE 2

| Left boundary | Right boundary | Range length | Features | Num SNPs | Notes |
|---|---|---|---|---|---|
| 1350800 | 1391400 | 40,600 | CP-933C, CP-933X | 416 | a |
| 1403500 | 1411000 | 7,500 | CP-933X | 68 | a |
| 1869000 | 1871000 | 2,000 | CP-933P | 17 | a |
| 1913000 | 1921500 | 8,500 | CP-933P | 137 | a |
| 2324000 | 2336000 | 12,000 | CP-933U | 63 | a |
| 2347000 | 2353000 | 6,000 | CP-933U | 204 | a |
| 2413500 | 2423000 | 7,500 | his operon | 338 | b |
| 2439500 | 2457000 | 17,500 | O-antigen, colanic | 2,426 | b |
| | | | acid loci | | |
| 2596500 | 2660000 | 63,500 | CP-933V | 115 | a |
| TOTALS | | 165,100 | | 3,784 | |

^{a}Disintegration of cryptic prophage that are no longer under selection, and occupation of the site by an alternate related prophage may be most plausible explanations. However, mapping artifacts due to repeated sequences may be a possibility.
^{b}The his, O-antigen, and colanic acid loci all appeared to have co-transferred during the event that converted O55 to O157. Estimation of the recombination breakpoints from this analysis may be performed. The present E. coli O55 sequence in the 67 kb region sequenced by Iguchi (Microbiology. 2008 Feb; 154(Pt 2): 559-70) (not this whole region) differs from E. coli O55:H7 TB182 by only 24 SNPs, indicating accuracy of the sequence assembly.
Average SNP rate in these regions is 2.3%. When calculating divergence times and other factors atypical regions may be omitted from the analysis. SNP rate in the rest of the genome is only about 0.06%, 38-fold lower than for these regions.

In some embodiments sequences specific and unique to E. coli O55:H7 genome can be used to identify E. coli O55:H7 or distinguish E. coli O55:H7 from all other E. coli and Shigella genomes. One example method used to identify E. coli O55:H7 specific sequences is outlined in Example IV.

Prior to the teachings of the present disclosure, ‘O-islands’ where described as nucleic acid sequence regions specific and unique to and found only in E. coli O157:H7 serotype, (Perna, N. T., et al., (2001) Nature 409(25):529-533). O-islands regions total 1.34 megabases. Comparison of the genome of E. coli O157:H7 with that of E. coli O55:H7 unexpectedly revealed that E. coli O55:H7 serotype also contained many of the O-islands, most of which were virtually identical to those of E. coli O157:H7. Therefore, prior to the teachings of the present disclosure, a definitive identification of E. coli O157:H7, and likewise of E. coli O55:H7, was difficult due to genome sequence similarity, even in supposedly E. coli O157:H7 serotype specific regions, i.e., O-islands.

Embodiments of the present disclosure have identified serotype specific and unique DNA sequences for E. coli O55:H7 (e.g., but not limited to, SEQ ID NO:66, SEQ ID NO:252, SEQ ID NO:1113, SEQ ID NO:1461, SEQ ID NO:1, SEQ ID NO:2, SEQ ID NO:3, SEQ ID NO:4 and SEQ ID NO:5) which were utilized for an assay design (described in Example V) and the subsequent detection of E. coli O55:H7 and not E. coli O157:H7 by amplification (PCR), hybridization and other molecular biology techniques as known to one skilled in the art.

Five E. coli O55:H7 specific sequences, covering a sum of 1,124 nucleotides were found using the analysis of Example IV. These sequences are shown in FIG. 1 and in the submitted sequence listing. Further analysis, including PCR and sequencing from a diverse panel of E. coli O55:H7 strains is currently underway. Assays targeting these sequences are also being screened against a large panel of E. coli non-O55:H7 serotypes to empirically validate specificity.

In some embodiments, the sequences designated by SEQ ID NOS:1-5 and SEQ ID NO:66, SEQ ID NO:252, SEQ ID NO:1113, SEQ ID NO:1461, are signature sequences against which E. coli O55:H7-specific diagnostic assays have been designed in the present disclosure. No comparable sequences were found in the GenBank database (release 175.0) and unexpectedly, appear to be specific and unique to only E. coli O55:H7. The coordinates of E. coli O55:H7 specific sequences are provided in Table 3.

TABLE 3

| Signature | Contig | | |
|---|---|---|---|
| SEQ ID | SEQ ID | Contig left | Contig right |
| NO: | NO: | coordinate | coordinate |
| 1 | 66 | 5504 | 5652 |
| 2 | 252 | 1887 | 2650 |
| 3 | 1113 | 1617 | 1676 |
| 4 | 1113 | 1734 | 1794 |
| 5 | 1461 | 23329 | 23418 |

In some embodiments, SEQ ID NOS:1-5 represent nucleic acid sequence substrings selected from nucleic acid sequences set forth in SEQ ID NO:66, SEQ ID NO:252, SEQ ID NO:1113 (SEQ ID NOS:3-4), and SEQ ID NO:1461 respectively. Any of these sequences as well as complements and sequences comprising at least 90% nucleic acid sequence identity thereof can be used to identify and/or distinguish E. coli O55:H7 from other E. coli serotypes, Salmonella sp., and Shigella genomes. In some embodiments, a sequence having at least 25 contiguous nucleotides of these sequences as well as complementary sequences and sequences comprising at least 90% nucleic acid sequence identity to SEQ ID NOS:1-5 and SEQ ID NO:66, SEQ ID NO:252, SEQ ID NO:1113 (SEQ ID NOS:3-4), and SEQ ID NO:1461 may also be used to identify and/or distinguish E. coli O55:H7 from other E. coli serotypes, Salmonella sp., and Shigella genomes.

Assays used for the detection and identification of E. coli O55:H7 may include, but are not limited to, use of an oligonucleotide sequence of the disclosure for hybridization, and/or as a primer pair used for PCR, and/or possibly in conjunction with a probe for real-time PCR. The length of an oligonucleotide probe and/or primer sequence may be as few as 10, at least 15, at least 20, at least 25, and up to 40 nucleotides in length. Use of larger than 40 nucleotide oligonucleotides are also contemplated. Design of sequences for hybridization detection and PCT are may be done by one of skill in the art in light of the teachings of this disclosure, such as for example the unique sequences of E. coli O55:H7.

Some exemplary probe and/or primer sequences of the disclosure may comprise SEQ ID NO: 6, SEQ ID NO:7, SEQ ID NO:8, SEQ ID NO:9, SEQ ID NO:10, SEQ ID NO: 11, SEQ ID NO:12, SEQ ID NO:13, SEQ ID NO:14, SEQ ID NO:15, SEQ ID NO:16, SEQ ID NO: 17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, fragments thereof, at least 10 contiguous nucleotide sequences thereof and complements thereof. In some embodiments, the oligonucleotide sequence may be comprised in recombinant constructs as well as complements and sequences comprising at least 90% nucleic acid sequence identity thereof.

### Example IV

### Assembly of the E. coli O55:H7 PE704 Genome

Mate-paired SOLiD™ reads from E. coli O55:H7 PE704 genome were mapped against the E. coli O157:H7 EDL933 reference genome (Refseq Acc. NC_{—}002655.2) and from these a consensus O55:H7 genomic sequence was derived. Gaps where no consensus sequences could be determined were identified, and PCR primers were designed to flank these regions. Long-range PCR was attempted for a number of these regions, and when successful, the resulting amplicons were sequenced using primer walking and Sanger sequencing methods. Ungapped SOLiD™ consensus contigs and Sanger sequence reads were assembled using GAP4 (See, R. Staden, D. P. Judge and J. K. Bonfield. Managing Sequencing Projects in the GAP4 Environment. Introduction to Bioinformatics. A Theoretical and Practical Approach. Eds. Stephen A. Krawetz and David D. Womble. Human Press Inc., Totawa, N.J. 07512 (2003)), yielding a total of 1,662 contigs (SEQ ID NOs 33 through 1664, (attached in the Sequence Listing filed concurrently herewith). Some of the O55:H7 genomic regions with a large number of clustered sequence gaps, primarily corresponding to some prophages in the EDL933 reference genome, were not able to be spanned by long-range PCR and remain unfinished.

### Example V

### Identification of E. coli O55:H7 Specific and Unique Regions

An E. coli O55:H7 pseudochromosome was the single inclusion organism, i.e., the organism to be detected and also acted as the reference genome. The exclusion set (organisms to not be detected) consisted of 42 complete and near-complete E. coli and Shigella genomes. Table 4 is a list of the E. coli and Shigella genomes used as exclusion set.

TABLE 4

| Species | Strain | Serotype |
|---|---|---|
| E. coli | 101-1 | O?:H10 |
| E. coli | 53638 | O144 |
| E. coli | 536 | O6:K15:H31 |
| E. coli | APEC_O1 | O1:K1:H7 |
| E. coli | B171 | O111:NM |
| E. coli | B7A | O148:H28 |
| E. coli | B | unknown |
| E. coli | CFT073 | O6:H1 |
| E. coli | E110019 | O111:H9 |
| E. coli | E22 | O103:H2 |
| E. coli | E24377A | O139:H28 |
| E. coli | ec4024 | O157:H7 |
| E. coli | ec4042 | O157:H7 |
| E. coli | ec4045 | O157:H7 |
| E. coli | ec4076 | O157:H7 |
| E. coli | ec4113 | O157:H7 |
| E. coli | ec4115 | O157:H7 |
| E. coli | ec4196 | O157:H7 |
| E. coli | ec4206 | O157:H7 |
| E. coli | ec4401 | O157:H7 |
| E. coli | ec4486 | O157:H7 |
| E. coli | ec4501 | O157:H7 |
| E. coli | ec508 | O157:H7 |
| E. coli | ec869 | O157:H7 |
| E. coli | Sakai | O157:H7 |
| E. coli | EDL933 | O157:H7 |
| E. coli | F11 | O6:H31 |
| E. coli | HS | O9 |
| E. coli | MG1655 | O- |
| E. coli | SECEC_SMS-3-5 | unknown |
| E. coli | UTI89 | O18:K1:H7 |
| E. coli | W3110 | O- |
| S. boydii | BS512 | unknown |
| S. boydii | Sb227 | unknown |
| S. dysenteriae | 1012 | unknown |
| S. dysenteriae | M131649 | unknown |
| S. dysenteriae | Sd197 | unknown |
| S. flexneri | 2457T | 2a |
| S. flexneri | 301 | 2a |
| S. flexneri | 8401 | 5 |
| S. sonnei | 53G | unknown |
| S. sonnei | Ss046 | unknown |

The 42 genomes described above were aligned against the E. coli O55:H7 pseudochromosome sequence using the MUMmer program. Pairwise alignments between each of the inclusion/reference (E. coli O55:H7) and exclusion genomes were computed using the nucmer program within the MUMmer suite (Delcher, A. L., et al. Nuc. Acids Res. 27:2369-2376 (1999)). Significant hits between the exclusion genomes and the E. coli O55:H7 pseudochromosome (greater than or equal to 80% identity over at least 50 nt) were extracted from the matching output. The union of all significant exclusion genome hits on the E. coli O55:H7 genome was determined utilizing a Perl program to parse MUMmer output files and calculate interval ranges, as would be known to one of skill in the art in light of this disclosure. The signatures of interest were those sequences present in E. coli O55:H7, but not present in any exclusion organism. Expressed in mathematical terms, it is the difference between the intersection of inclusion hits and the union of exclusion hits.

27 putative E. coli O55:H7 specific and unique sequences of at least 60 nucleotides in length that did not consist of predominantly N bases (i.e., undetermined sequence) were identified. These 27 sequences covered 23,470 bases of the E. coli O55:H7 genome. 22 of the 27 candidate sequences were eliminated from further consideration by screening using BLASTN against the GenBank non-redundant database. Sequences having a BLASTN hit with more than 80% identity over more than 50 nucletoides to an organism other than E. coli O55:H7 were eliminated from consideration. About half of the 22 eliminated sequences were in the E. coli O55:H7 0-antigen biosynthesis cluster, and were therefore conserved in E. coli serotype O55:H6 strains. Other eliminated candidates shared sequence with E. coli serotypes O127:H6, O7:K1, and O17:K52:H18, and E. fergusonii.

The remaining five E. coli O55:H7 specific sequences, covering a sum of 1,124 nucleotides, are described in FIG. 1.

### Example V1

### Assays to Specifically Detect E. coli O55:H7

Exemplary real-time PCR assays were designed from specific and unique and specific E. coli O55:H7 sequence regions, SEQ ID NOS:1-5 and SEQ ID NO: 66, SEQ ID NO: 252, SEQ ID NO: 1113, SEQ ID NO: 1461. These identified E. coli O55:H7 target sequences were used to design primers and probes for real-time PCR assays. Programs for assay design include Primer3 (Steve Rozen and Helen J. Skaletsky (2000) “Primer3” on the World Wide Web for general users and for biologist programmers as published in: Krawetz S, Misener S (eds) Bioinformatics Methods and Protocols: Methods in Molecular Biology. Humana Press, Totowa, N.J., pp 365-386), Primer Express® software (Applied Biosystems), and OLIGO 7 (Wojciech Rychlik (2007). “OLIGO 7 Primer Analysis Software”. Methods Mol. Biol. 402: 35-60)). The subsequently designed PCR primers and probes for use in assays by real-time PCR can detect unambiguously, specifically and with great sensitivity E. coli O55:H7 and not O157:H7.

### Example VII

### Some Exemplary Nucleic Acid Sequences of the Disclosure

TABLE 5
Exemplary Sequences Described in Some
Embodiments with Corresponding SEQ ID NOS:

AAAAGTGCTCATCGTAAAATATCCTCTATACTGGTAAGTCCTTCATTA
TTTAGCCAGATCATGGCGTCATCCGGCAACTTGCTGTCTGGTTTGGCG
TTTTTGAGAGAGGAACTCAGTCGCTTAATCCACATTGTCACCTCAGTA
ATGCG (SEQ ID NO: 1)
CACAACTTAGCATCGAAGGCCTATTGTTTTCGACAGGAGGCCATCGAA
ATGACACTATGGTACCGTCTTTACCCACCGCCTTCTGCTAAGTTTAGG
AATACAAGATGTTGCTGCCGTTAAAAAATAGTCGTAATTTCTATTAGA
ATTTGGGCAATATAACCAGCACATAGATAAGGGAGTCGTTTTATGCAC
GAACTATTTGTGCTGGTTTTGAGCACCTGTGCTAGCCTCAGCAATATG
TCAGGTTGTTCAGTGGACGTAGTGAATGTCAACACCACAAAAGAGCCG
GTAAACGTCTTTTATAGCAGGAAAGACTGCGAAGAAAGCATGAAAAAC
ATTATGCTAAATCATGCTCTATACCATGAAGTATCAGGCAGAGAGCCA
TATATGGCAAAGTGCGAACAAGTATTTGTCTCAAAAAGTTTAATGAAA
TAATTGGCCAAAAACGACATATAACAAACTCTTATAGGAGCTTAACGT
GGAAGATTACTTAGTTTTTGGTTTAGGTTATGAAGGCGATATAAAGAA
CGATGAAGCAGGGCTTGATAAGATAAGCGTGGTAACGAAATCGGTAGT
GCGTTCTACCAATTCCAATGAACCAGTGGTTTACCAAGTGACTGAGTT
TAAAGAATTTAATGTTGTGAGACATCAAGCGTTTGATAGTGAATACTA
CAACATAGCATTCGATGTCTTACCATCCCGCGTCCGTATTGATGCTGC
AATCCGTAAATATCACCCCAAGAAATCCTCCTCAGCTTAGTAAG
(SEQ ID NO: 2)
TGTTGCCCTGGCCATCCAGGAAAAATATGGTTTCCCACCGCTATATAA
TGATTTCCGCAA (SEQ ID NO: 3)
CAAGCTACCTGTCGACTATCCCGTAAAATACACGGACCTTTTCACTCA
CATACACGCCAAT (SEQ ID NO: 4)
TTTATACACGGATCCTGTGTGCCGTGGACCGCCGGTTTATCCCCGCTG
GCGCGGGGAACACCACAAACCGCCCATCTTCCCGATTACTGC
(SEQ ID NO: 5)
GGTAAGTCCT TCATTATTTA GCCAGATCA (SEQ ID NO: 6)
AGCGACTGAG TTCCTCTCTC AA (SEQ ID NO: 7)
GATCCTGTGT GCCGTGGA (SEQ ID NO: 8)
CGGGAAGATG GGCGGTTT (SEQ ID NO: 9)
ACGAACTATT TGTGCTGGTT TTGAG (SEQ ID NO: 10)
CTCTTTTGTG GTGTTGACAT TCACT (SEQ ID NO: 11)
AGTGGTTTAC CAAGTGACTG AGTTT (SEQ ID NO: 12)
GGGATGGTAA GACATCGAAT GCTAT (SEQ ID NO: 13)
GAAGGCCTAT TGTTTTCGAC AG (SEQ ID NO: 14)
AGAAGGCGGT GGGTAAAGAC (SEQ ID NO: 15)
GCAAAGTGCG AACAAGTATT TGTCT (SEQ ID NO: 16)
ACTAAGTAAT CTTCCACGTT AAGCTCCTA (SEQ ID NO: 17)
GAATACAAGA TGTTGCTGCC GTTAA (SEQ ID NO: 18)
CGTGCATAAA ACGACTCCCT TATCT (SEQ ID NO: 19)
GGTTATGAAG GCGATATAAA GAACGATGA (SEQ ID NO: 20)
CGCACTACCG ATTTCGTTAC CA (SEQ ID NO: 21)
GAAAGACTGC GAAGAAAGCA TGAAA (SEQ ID NO: 22)
CATATATGGC TCTCTGCCTG ATACTT (SEQ ID NO: 23)
AAGTTGCCGG ATGACGC (SEQ ID NO: 24)
CCGGTTTATC CCCGCTGGC (SEQ ID NO: 25)
ACGTCCACTG AACAACC (SEQ ID NO: 26)
AACGCTTGAT GTCTCACAAC A (SEQ ID NO: 27)
TAGTGTCATT TCGATGGCCT C (SEQ ID NO: 28)
CCAAAAACGA CATATAACAA AC (SEQ ID NO: 29)
TAGAATTTGG GCAATATAAC C (SEQ ID NO: 30)
CAGGGCTTGA TAAGATAAG (SEQ ID NO: 31)
ATGGTATAGA GCATGATTTA GC (SEQ ID NO: 32)
ANATTCTGCGGGAGAGCCCCGTTGAAAACAGGAAAGTTTTTAACCTGA
GATTGTTAAAGATATATTACAGATTAATGATATTCTTAAAATGTGGTA
ATTTATTAAATCTGTAATAAAAGCGTAAACAACTGCCGCTAGGCTTGC
TGATCCCGCGCAACAAAACGCCATGCTTTGCTCGCAGATGGTTGGCAA
CCGACGACAGTCCTGCTAAAACGTTCGTTTGATATCATTTTTCCTAAA
ATTGAATGGCAGAGAATCATGAGTGACAGCCAGACGCTGGTGGTAAAA
CTCGGCACCAGTGTGCTAACAGGCGGATCGCGCCGCCTGAACCGTGCC
CATATCGTTGAACTTGTTCGCCAGTGCGCGCAGTTACATGCCGCCGGG
CATCGGATTGTTATTGTGACGTCGGGCGCGATCGCCGCCGGACGTGAG
CACCTGGGTTACCCGGAACTGCCAGCGACTATCGCCTCGAAACAACTG
CTGGCGGCGGTAGGGCAGAGTCGACTGATTCAACTGTGGGAACAGCTG
TTTTCGATTTATGGCATTCACGTCGGGCAAATGCTGCTGACTCGTGCT
GATATGGAAGACCGTGAACGCTTCCTGAACGCCCGCGACACCCTGCGT
GCGTTGCTCGATAACAATATCGTTCCGGTAATCAATGAGAACGATGCT
GTCGCTACGGCAGAGATTANNNNCGGCGATAACGACAACCTTTCTGCA
CTGGCGGCGATTCTGGCGGGTGCCGATAAACTGTTGTTACTGACCGAT
CAAAAAGGTTTGTATACCGCTGACCCGCGCAGCAATCCGCAGGCAGAA
CTGATTAAAGATGTTTACGGCATTGATGACGCACTGCGNGCGATTGCT
GGTGACAGCGTTTCAGGCCTCGGAACTGGCGGCATGAGTACCAAATTG
CAGGCCGCTGACGTGGCTTGCCGTGCGGGTATCGACACCATTATTGCC
GCGGGCAGCAAGCCGGGCGTTATTGGTGATGTGATGGAAGGCATTTCC
GTCGGTACGCTGTTCCATGCCCAGGCGACTCCGCTTGAAAACCGTAAA
CGCTGGATTTTCGGTNNNNCGCCTGCGGGTGAAATCACGGTAGATGAA
GGGGCAACCGCCGCCATTCTTGAACGCGGCAGCTCCCTGTTGCCGAAA
GGNNNNAAAAGCGTGACTGGCAACTTCTCGCGTGGTGAAGTCATCCGC
ATTTGTAACCTCGAAGGTCGCGATATCGCCCACGGCGTCAGTCGTTAC
AACAGCGATGCATTACGCCGTATTGCCGGACACCACTCGCAAGAAATT
GATGCAATACTGGGATATGAATACGGCCCGGTTGCCGTTCACCGTGAT
GACATGATCACCCGTTAAGGAGCAGGCTGATGCTGGAACAAATGGGCA
TTGCCGCGAAGCAAGCCTCGTATAAATTAGCGCAACTCTCCAGCCGCG
AAAAAAATCGCGTGCTGGAAAAAATCGCCGATGAACTGGAAGCACAAA
GCGAAATCATCCTCAACGCTAACGCCCAGGATGTTGCTGACGCGCGAG
CCAATGGCCTTAGCGAAGCGATGCTTGACCGTCTGGCACTGACGCCCG
CACGGCTGAAAGGCATTGCCGACGATGTACGTCAGGTGTGCAACCTCG
CCGATCCAGTGGGGCAGGTAATCGATGGCGGCGTACTGGACAGCGGCC
TGCGTCTTGAGCGTCGTCGCGTACCGCTGGGGGTTATTGGCGTGATTT
ATGAAGCGCGCCCGAACGTGACGGTTGATGTCGCTTCGCTGTGCCTGA
AAACCGGTAACGCGGTGATCCTGCGTGGTGGCAAAGAAACCTGTCGCA
CTAACGCGGCAACGGTGGTGGTGATTCAGGACGCCCTGAAATCCTGTG
GCTTACCGGCGGGTGCCGTGCAGGCGATTGATAATCCTGACCGTGCGC
TGGTCAGTGAAATGCTGCGTATGGATAAATACATCGACATGCTGATCC
CGCGCGGCGGGGCTGGTTTGCATAAACTGTGCCGCGAGCAGTCGACGA
TCCCGGTGATCACAGGTGGTATAGGCGTATGCCATATTTATGTTGATG
AAAGTGCAGAGATTGCTGAAGCCCTGAAAGTAATCGTCAATGCGAAAA
CTCAGCGTCCGAGCACATGTAATACGGTAGAAACGTTGCTGGTGAATA
AAAACATCGCCTATAGCTTCCTGCCCGCATTAAGCAAACAAATGGCGG
AAAGCGGCGTGACGTTACACGCAGATGCATCTGCGCTGGCGCAGTTGC
AGACAGGCCCTGCGAAGGTGGTGGCGGTTAAAGCCGAAGAGTATGACG
ATGAGTTTCTGTCATTAGATTTGAACGTCAAAATCGTCAGCGATCTTG
ACGATGCCATCGCCCATATTCGTGAACACGGCACGCAACACTCCGATG
CGATCCTGACCCGCGATATGCGCAACGCCCAGCGTTTTATTAACGAAG
TGGATTCGTCCGCTGTTTACGTTAACGCCTCTACGCGTTTTACCGACG
GCGGCCAGTTTGGACTGGGTGCGGAAGTGGCGGTAAGCACACAAAAAC
TCCACGCNCGTGGCCCAATGGGGCTGGAAGCACTGACCACTTACAAGT
GGATCGGCATTGGTGATTACACCATTCGTGCGTAAATAAAACCGGGTG
ATGCAAAAGTAGCCGTTTGATTCACAAGGCCATTGACGCATCGCCCGG
TTAGTTTTAACCTTGTCCACCGTGATTCACGTTCGTGAACATGTCCTT
TCAGGGCCGATATAGCTCAGTTGGTAGAGCAGCGCATTCGTAATGCGA
AGGTCGTAGGTTCGACTCCTATTATCGNCACCACTTAAATCAATAAGT
TACCTCGCATTTAAGTAAACCACGTTCTCCTCTTGTGCCGTATTTGTG
CCATTGCGACTTATAATCGCATCGATTTTGCTCGCGTGCTCGGTGAGA
TGCCCGGCTGAAAGGTGGGCGTATCTTTGAACCATTTCGAGAGTTTCC
CATCCTCCCATCTCTTTAAGTGCAAGAAGAGAGACACCGGACTGAACC
AGCCAGCTTGCCCAGGTATGCCTCAGGTCATGGAAGCGGAAGTTGCTA
ATGCCTGCCCGCTTTAACGCTCCTTTCCATGCCTTGTTGCTGTCGGTT
CTCATCTTCCTTACCGCTGCTGTTTTTGTTCCGTCGCTTCGGTAGGCA
GGTTTGGTGTGGACAAATACCCATCTCTTATGGAGCCCCTGCTGTTTT
CTTAATATCTGGCATGCGGTTTCGTTAAGAGGAACTCCGATCGCATTG
CCAGCTTTTGTTTCATCAGGGTGCATCCATGCCATTTTCTTATCCAGA
TCGACCTGTGACCACTCAAGGTCTGTAACGTTGGAGCGGCGAAGACCT
GTAGTGATTGCGAACATGACCACAGGGAAGAAATGAGGAGCAATTTCT
GCAAACAGGCGCTTCGATTCCTCCTCTGTAAGCCATCTGATTCGTCCA
TTCTTAACGCGTGGTGTTGATATTTTTGGCGCCCTGTCAAGCCATCCA
CATTCAACAGCCATATTGAGAATAGCGCGAAGTATTGCCAGATGCCGC
GTCTTCGTTCCTTTGCTTGCCAGCTTTGGTTTATACTCCGGCACTGGC
TTGCCAAGCCGCAAACACCTGTCCCGGCTCATCTCCCAGTTCAGGCGA
TGGCGGCGGTTTTCCATCCCGTCTACCGCCTCCATTATTTTTTCTGTT
GTTATGTCAGAGAGAATGGTTTCTCTGAAGTGCAACATCCAGAACGAT
ATAATGCTCTTGTCATCATCAATGGACTTCTTATCCGATTTCTCACGC
AGCCACCGTATGCAGGCTTCCTTGAATAGCTTTTTCGGCGATTCCCCG
AGATTTTTTACTCTCCACGCCTCTGCTTTCAGACGATCGTGAAGTTCT
TGCGCTTGCCTTTTGTCCGATGTTTCAAGAGAGCGTCTAACTCTTGAT
CCATCTTGCGCGACGAAATCGCAGTGCCATGTGCCACCGCGCAGTTTG
ATTGACATGCTTTAACCTCCTGCACATCAACCGCATTCACCGCGCTAT
TGTGTCTCACAGACTTAAGCGCCGCAATGCAGTCTGACTTGCAAATGC
GATATGGGCTTTTAGGTTTATCTGGATTTATCTTTGCGGCCTGAAGTC
GTCCACTTCGTATCCACTGCGTGATAGTGCCTTTGTCTACCTTCAGAT
ACGACGCAGCCTCTTCACGAGTGAAGATTTCTTCTTCCACTTGGAGTC
TCCATTTATTGGATTGACATGATTGCTGTAGGTCTGGATATCTTGAGA
AGCTGGCAGGCCTCATCGAGTGTGAGGCTGTGGTTAGTCCTTGCGTAA
'CTCGCTAATTCTTCTGTAAGTCTCTGGTGCTTTGTTTCCGTGTATCT
TCATTTCAGACTTCAACAGAGCAACGAGAGAATCCCATTCGTTGAGGA
TTCCTTTGAATGCCGGAACGCGCTTTGCAACCTTGTCGAATGAATCTC
TGATTTCTGGAATCTGCTCAACAAGTGCAACGCATCGTCTGAAATCGG
CTGCGTCATGTGGAGCACCGAAGCTATGACCATAGATATTCTTTTTCA
GGCCACATGCGATTGAGGCAAGAGTTGCGCTACTGATGCCGACATCGC
CAGTCGATTGCCATTTCAAAACCTTCATAGCCAAATCTGACATTTCTT
GTCTCCATAAAACAAAACCCGCCGTAGCGAGTTCAGATAAAAGAAATC
CCCGCGAGTGCGAGGATTGTTATTCACCTTTGACGGCAAGTTGCAGGT
TAGCCACGGTTAACTCCTTGCTGTGTGGCGTCGAGTAATAAATCCCAC
AACCGCTGAGAGCAATTTCCGCATGTACAGCGTTGACCCACAACGTTT
ATCGCGTTGCTGATTTCCGGCGTTACCTGTTTAGGAACCATAACCCAA
CCATCCGGAGTTACCGGAAGCGAGAACGGCAGCACATCTCTGTGAACA
AGTTTTTGCTGTGACAGGTTATCCAGAACTTTCTGTACTGCTGCATCA
CCGAATACACCAAGCGCATCTGCCATAACTCCTACAACCTGATAAGCC
TCAGCGCATACCGTGGATAAACCATCCGGAGTTACCGGAGAGTTGCCG
GGTTCTTTAATGTGCAAGCGAGGCTCACCATCTTTTGGCTCAGGCCAC
TGGCGCTCCATGTTGATCTTCAATTTATCTTCCATAGCAGCGGTAATT
TCAGCATCGCTGATGCCGGCACGGCGCTGTGCATCCCACAACAGAAAC
TGCATATCAGCCCACTCGCTAAGATCGTCTGGTTCGGCTGCGGCTTCC
AGAGCCTCTTTTGAGAGGTGTTTCAGTGGACCAATGGGGCCAACGCAG
CCAAATGTGGAGTCAGACCATTTGGCATGCTCGTGGCGAATCTGTTCG
CGTTCCAGTGAGGCGAGAGCGATTTTAAATGCTGTAAGCATGTGTGCT
TGATCGTTGTCGAGTCCGAATGGCATTTCATCTCGTGCTGACTCAATA
CTGGTAATCGTATTTTGTAGCCATTCTTTGGTAAAAGTGCTCATCGTA
AAATATCCTCTATACTGGTAAGTCCTTCATTATTTAGCCAGATCATGG
CGTCATCCGGCAACTTGCTGTCTGGTTTGGCGTTTTTGAGAGAGGAAC
TCAGTCGCTTAATCCACATTGTCACCTCAGTAATGCGCCTGTCTTTGC
CTTCCAGCTCAACACGCAGCTTCCCTACCGTTAGCGCAATTTCCTCGC
TCTCTTGGTCGCGTGATTTGATGTATTGCTGGTTTCTTTCCCGTTCAT
CCAGCAGTGCCAAGACGGTAGCAGGATTGGCTGCGGCGATGAATTCAG
CATTGGCCTGCTGTTCCATTTGGAAATCTTCATCGAAACCGCTTTCTG
GATGCGCTCCTTCAATTCTGCAAATAGGAATATATCCAGCAACTTCAC
GATGAATTAGCGCATCATCACCATCAAATCGGCTCTCTCCATATTCGA
GCGACCGCTCACCACACGTTGCTTTTTCTGCCGCCTCACGCAGTGCCT
GAGAGTTAATTTCGCTCACTTCGAACCTCTCTGTTTACTGATAAGCTC
CAGATCCTTCTGGCAACTTGCACAAGTCCGACAACCCTGAACGGCCAG
ACGTCTTAGTTCATCTATCGGATCGCCACACTCACAACAATGAGTGGC
AGATATAGCCTGGTGGTTCAGGCGGTGCATTTTTATTGCTGTGTTGCG
CTGTAATTCTTCAATTTCTGATGCTGAATCAATGATGTCTGCCATCTT
CCATTAATCCCTGAATTGTTGGTTAATACGCTTGAGAGTGAATGCGAA
TAATAAAAAAGGAGCCTGTAGCTCCCTGATGATTTTGCTTTTCATGTT
CACCGTTCCTTAAAGACGCCGTTTAACATACCGATTGCCAGACTTAAG
TGAGTCGGTGTGAATCCCATCAGCGTTACCGTTTCGCGGTGCTTCTTC
AGTACGCTACGGCAAATGTCATCGACGTTTTTATCCGGAAACTGCTGT
CTGGCTTTTTTGATTTCAGAATTAGCCTGACGGGCAATACTGCGAAGG
GCGTTTTCTTGCTGAGGTGTCATTGAACAAGTCCCATGTCGGCAAGCA
TAAGCACACAGAATATGAAGCCCGCTGCCAGAAAAATGCATTCAGTGG
TTGTCATACCAGGTCTCTCTCATCTGCTTCTGCTTTCGCCACCATCAT
TTCCAGCTTTTGCGAAAGGGATGTGGCTAACGTATGAAATTCTTCGTC
TGTTTCTACTGGTATTGGCACAAACCTGACTCCAATTTGAGCAAGGCT
ATGTGCCATCTCAATACTCGTTCTTAACTCAACAGGAGATGCTTTGTG
CATATCGCCTCCCGTTTATTATTTATCTCCTCAGCCAGCCGCTGGGCT
TTCAGCGGATTTCGGATAACAGAAAGGCCGGGAAATACCCAGCCTCGC
TTCGTAACGGAGTAGACGAAAGTGATCGTGCCTACGCGGATATTATCG
TGAGGATGCTTCATCGCCATTGCTCCCCAAATACAAAACCAATTTCAG
CCAGTGCCTCGTCCATTTTTTCGATGAACTCCGGCACCATCTCGTCAA
AACCCGCCATGTACTTTTCATCCCGCTCAACCACGACATAATGCAGGC
CTTCACGCTTCATACGCGGGTCATAGTTGGCAAAGTACCAGGCATCTT
TTCGCGTCACCCACATGCTGTACTGCACCTGGGCCATGTAAGCCGACT
TTATGGCCTCGAAACCACCGAGCCTGAACTTCATGAAATCCCGGGAGG
TAAACGGGCATTTCAGCTCAAGGCCGTTGCCGTCACTGCATAAACCAT
CGGGAGAGCAGGCGGTGCGCATACTTTCGTCGCGATAGATGATCGGGG
ATTCAGTAACATTCACGCCGGAAGTGAATTCAAACAGGGTTCTGGCGT
CGTTCTCGTACTGTTTTCCCCAGGCCAGCGCCTTAGCATTAACTTCCG
GAGCCACACCGGTGCAAACCTCAGCCAGCAGGGTGTGGAAGTAGGACA
TTTTCATGTCAGGCCACTTCTTTCCTGAGCGGGGCTTTGCTATCACGT
TGTGAACTTCTGAAGCGGTGATGACGCCGAGCCGTAATTTGTGCCATG
CATCATCCCCCTGTTCGACAGCTCTCACGTCGATCCCGGTACGCTGCA
GGATAATGTCCGGTGTCATGCTGCCACCTTCTGCTCAGTGGCTTTCTG
TTTCAGGAATCCAAGAGCTTTCACTGCTTCGGCCTGTGTCAGTTCTGA
CGATGCGCGAATGTCGCGGCGAAATATCTGGGAACAGAGCGGCAATAA
GTCGTCATCCCATGTTTTATCCAGGGCGATTAGCAGAGTGTTAATCTC
CTGCATGGTTTCATCGTTAACCGGAGTGATGTCGCGTTCCGGCTGGCG
TTCTGCAGTGTATGCAGTATTTTCGACAATGCGCTCGGCTTCATCCTT
GTCATAGATACCAGCAAATCCGAAGGCCAGACGGGCACACTGAATCAT
AGCTTTATGCCGTAACATCCGTTTAGGATGCGACTGCCACGGCCCCGT
GATTTCTCTGCCTTCGCGGGTTTTGAATGGTTCGCGGCGGCATTCATC
CATCCATTCGGTAACGCAGATCGGATGATTACGGTCCTTGCGGTAAAT
CCGGCATGTACAGGATTCATTGTCCTGCTCAAAGTCCATGCCATCAAA
CTGCTGGTTTTCATTGATGATGCGGGACCAGCCATCAACGCCCACCAC
CGGAACGATGCCGTTCTGCTTATCAGGGAAGGCGTAAATTTCTTTCGT
CCACGGATTAAGGCCGTACTGGTTGGCGACGATCAACAATGTGATGAA
CTGCGCATCGCTGGCATCGCCTTTAAATGCCGTCTGGCGAAGAGTGGT
GATCAGTTCCTGTGGGTCGACAGAATCCATGCCGACACGTTCAGCCAG
CTTCCCTGCCAGCGTTGCGAGTGCTGTACTCATCCGTTTTATACCTCT
GAATCAATATCAACCTGATGGTGAGCAATGGTTTCAACCATGTACCGG
ATGTGTTCTGCCATGCGCTCCTGAAACTCAACATCGTCATCAAACGCA
CGGGTAATGGCTTTTTTGCTGGCCCCGTGGCGTTGCAAATGACCGATG
CATAGCGATTCAAACAGGTGCTGGGGCAGGCCTTTTTCCATGTCGTCT
GCCAGTTCTGCCTCTTTCTCTTCACGGGCTATCTGCTGGTAGTGACGC
GCCCAGCTCTGAGCCTCAAGACGATCCTGAATGTAATAAGCGTTCATG
GCCGAACTCCTGAAATAGCTGTGAAAATATCGCCCGCGAAATGCCGGG
CTGATTAGGAAAACAGGAAAGGGGTTAGTGAATGCTTTTGCTTGATCT
CAGTTTCAGTATTAATATCCATTTTNNANNNGCNNNNACGGCTTCACG
AAACATCTTTTCAT (SEQ ID NO: 66)
CTGAGATATTCAATTTTCCAGTGCCGGATGAGGCACAAAAGGAGCGGC
GCGTGGCAGATCTCGATGATGGTTATACGCGCATTGCAAATGAGTTGC
TGGAAGCTGTGATGCTGGCCGGATTAACACAGCACCAGCTTCTGGTCT
TCCTGGCTGTCATGCGCAAAACATATGGCTTTAATAAAAAACTGGATT
GGGTGAGCAACGAGCAACTTTCCGAATTGACCGGGATATTGCCGCACA
AGTGTTCTTCTGCAAAAAGCGTTCTGGTAAAGCGTGGGATTCTTATTC
AGAGCGGGCGGAATATCGGCATTAATAATGTGGTCAGTGAATGGTCAA
CATTACCCGAATCAGGTAAGAAAAATAAAGTTTACCTGAAANNGGTAA
NNTTACCTGAATCAGGTAAGAAAAGTTTACCCAAATCAGGTAAAGGCG
TTTACCCGAATCAGGTAAACACAAAAGACAAACTAACAAAAGACAATA
TAAAACCTTTTTCGTCCGAGAATTCTGGCGAATCCTCTGACCAACCAG
AAAACGATCTTCCTGTGGAGAAACCAGATGCTGCAATTCAGAGCGGCA
GCAGGTGGGGGACAGCAGAAGACCTGACCGCCGCAGAGTGGATGTTTG
ACATGGTGAAGACCATCGCGCCATCAGCCAGAAAACCGAATTTTGCAG
GGTGGGCTAACGATATCCGCCTGATGCGTGAACGTGACGGACGTAACC
ACCGCGACATGTGCGTGCTGTTCCGCTGGGCATGCCAGGACAACTTCT
GGTCCGGTAACGTGCTAAGTCCGGCCAAACTCCGCGACAAGTGGACCC
AACTCGAAATCAACCGTAACAAGCAACAGGCTGGCGTGACAGCCGGAA
AATCAAAACTGGACCTGACAAACACTGACTGGATTTATGGGGTGGATT
TATGAAAAACATCGCCGCACAGATGGTTAACTTTGACCGTGAGCAGAT
GCGCCGGATCGCCAATAACATGCCGGAACAGTACGACGAAAAGCCGCA
GGTACAGTTGGTAGCGCAGATCATCAACGGTGTGTTCAGCCAGTTACT
GGCAACTTTCCCGGCGAGCCTGGCTAACCGGGACCAGAACGAACTGAA
CGAAATCCGCCGCCAGTGGGTTCTGGCTTTCCGGGAAAACGGGATCAC
CACAATGGAACAGGTTAACGCAGGAATGCGCGTAGCCCGTCGGCAGAA
TCGACCATTTCTGCCATCACCCGGGCAGTTTGTTGCATGGTGCCGGGA
AGAAGCATCCGTTATCGCCGGACTGCCAANCGTCAGCGAGCTGGTTGA
TATGGTTTACGAGTATTGCCGGAAGCGAGGCCTGTATCCGGATGCAGA
GTCTTATCCGTGGAAATCGAACGCGCACTACTGGCTGGTTACCAACCT
GTACCAGAACATGCGGGCCAATGCGCTGACTGACGCGGAATTACGACG
CAAGGCTGCCGATGAACTGACCTGTATGGCAGCGCGAATTAACCGTGG
TGAGACGATACCTGAACCAGTAAAACAACTTCCTGTCATGGGCGGCAG
ACCTCTAAATCGTGTTCAGGCGCTGGCGAAGATCGCAGAAATTAAAGC
TAAGTTCGGACTGAAAGGAGCAAGTGTATGACGGGCGAAGAGGCAATT
ATTCATTACCTGGGGACGCATAATAGCTTCTGTGCGCCGGACGTTGCC
GCGCTAACAGGCGCAACAGTAACCAGCATAAATCAGGCCGCGGCTAAA
ATGGCACGGGCAGGTCTTCTGGTTATCGAAGGTAAGGTCTGGCGAACG
GTGTATTACCGGTTTGCTACCAAGGAAGAACGGGAAGGAAAGGTGAGC
ACGAATATGATTTTTAAGGAGTGTCGCCAGAGTGCCGCGATGAAACGG
GTATTGTTGGTATACACAACTTAGCATCGAAGGCCTATTGTTTTCGAC
AGGAGGCCATCGAAATGACACTATGGTACCGTCTTTACCCACCGCCTT
CTGCTAAGTTTAGGAATACAAGATGTTGCTGCCGTTAAAAAATAGTCG
TAATTTCTATTAGAATTTGGGCAATATAACCAGCACATAGATAAGGGA
GTCGTTTTATGCACGAACTATTTGTGCTGGTTTTGAGCACCTGTGCTA
GCCTCAGCAATATGTCAGGTTGTTCAGTGGACGTAGTGAATGTCAACA
CCACAAAAGAGCCGGTAAACGTCTTTTATAGCAGGAAAGACTGCGAAG
AAAGCATGAAAAACATTATGCTAAATCATGCTCTATACCATGAAGTAT
CAGGCAGAGAGCCATATATGGCAAAGTGCGAACAAGTATTTGTCTCAA
AAAGTTTAATGAAATAATTGGCCAAAAACGACATATAACAAACTCTTA
TAGGAGCTTAACGTGGAAGATTACTTAGTTTTTGGTTTAGGTTATGAA
GGCGATATAAAGAACGATGAAGCAGGGCTTGATAAGATAAGCGTGGTA
ACGAAATCGGTAGTGCGTTCTACCAATTCCAATGAACCAGTGGTTTAC
CAAGTGACTGAGTTTAAAGAATTTAATGTTGTGAGACATCAAGCGTTT
GATAGTGAATACTACAACATAGCATTCGATGTCTTACCATCCCGCGTC
CGTATTGATGCTGCAATCCGTAAATATCACCCCAAGAAATCCTCCTCA
GCTTAGTAAGCTTTGATTTTCCATTATCAACCAGCAATAATAATGTCC
TCGGAGCCTGAACAACTCCGGTGACTTCTGCGCTAAACGGGGACGTTT
ATGCGCACATACAATCCAAACTCTCTTCTCCCTTCACTGATGCAGAAA
TGCACCTGTGGTTCTTTGCATCCAACGTTTGACCTCTGCGGAGGTGAA
GCGTGAACCTCCCACAAGACGGTATCAAATTGCATCGCGGTAACTTCA
CTGCTATCGGCCAGCAGATCCAGNCTTATCTG (SED ID NO: 252)
CAGAAGCATTCCTCAATACAGAACTGACCTGGGATGGTATCCAGCAAC
CGCTGTTGGGCCATAAAGTGAATCCGTTTAAGGCGCTGTATAACCGCA
TCGATATGAAACAGGTTGAAGCACTGGTGGAAGCATCTAAAGAAGAAG
TGAAAGCCGCTGCCGCGCCGGTAACTGGCCCGCTGGCAGACGATCCGA
TTCAGGAAACCATCACCTTTGACGACTTCGCTAAAGTTGACCTGCGCG
TGGCGCTGATTGAAAACGCAGAGTTTGTTGAAGGTTCTGACAAACTGC
TGCGCCTGACGCTGGATCTCGGCGGTGAAAAACGCAATGTCTTCTCCG
GCATTCGTTCTGCCTACCCGGATCCGCAGGCACTGATTGGTCGTCACA
CCATTATGGTGGCTAACCTGGCACCACGTAAAATGCGCTTCGGTATCT
CTGAAGGCATGGTGATGGCTGCCGGTCCTGGCGGGAAAGATATTTTCC
GCTAAGCCCGGATGCCGGTGCTAAACCGGGTCATCAGGTGAAATAATC
TCCTTTCAAGGCGCTGTATCGACAGCGCCTTTTCTTTATAAATTCCTA
AAGTTGTTTTCTTGCGATTTTGTCTCTCTCTAACCCGCATAAATACTG
GTAGCATCTGCATTCAACTGGATAAAATTACAGGGATGCAGAATGAGA
CACTTTATCTATCAGGACGAAAAATCACATAAATTCTGGGCGGTTGAG
CAGCAGGGAAACGAGCTGCATATCAACTGGGGCAAAGTCGGCACTAAC
GGGCAAAGCCAGATAAAAAGTTTTGCGGATGCTGCGGCAGCGGCAAAA
GCGGAGCTTAAGCTGATCGCAGAGAAAACGAAAAAAGGCTATGTGGAA
AATGCTTCAGCAAACGTGCATATCCCCCCCATTACCAAAGCCACTCCT
GAAGTTGAAACTTCCCCTGAGAGTAAAAACCAACGCCCCTGGCTGGCT
GATGATGCGGTCATCGGCTAACTGACGATATTAATCGATTTGCTTTTC
CTCATCGCTCTCGCCCCAGAGAGATCAATTATCTGCGCAAAGACGGTG
AGATATGGAAGCGTATTGCAGATAACACTAGGGCATATGATCCCGACA
ACAATTACCGCTCTTATCCAGAAAACTGGCAGCAGGCTTTTGCTGAGT
TACAAATGCGAGTTCAGGGTAATCAACAGACAGGAAGTGCGCAATCTG
ATGCGGCATTGCTCTGGAGTTTCTGGAACTCTTACTCTACAGATGAAC
TGGTCGATGACTTAGTCATTCGCTGTGGTCTGGAAAGTGCCGTTGAAA
TCGCCCTTCTCGCTCTTCAACTTAGATATAAACCAGTAAAAGGCGCAG
TAACCACCACCATTCCACCTAATCACAAAGCAGAATCGCTACCTAGTT
GGCATCAACGTCTATGTCATCATCTTTCACTCGCTTCAGAAGATGAGT
GGCAACGCTGTGTAGATAAAGTACTCGCGGCTATTCCCTCGCTATCCC
CAGCACGGGAACCTTTTGCGGCGTTACTGCTCCCTGAACGCCCGGATA
TTGCCAATGCTATGGCGCTACGTTATGCAGACCAAAACGTTCCGGCGA
TCACCTGGTTAAGTATGATGGTAAGCGATGATGTTGCCCTGGCCATCC
AGGAAAAATATGGTTTCCCACCGCTATATAATGATTTCCGCAAATATC
TGGCTACGTTGCTGGCAAATAATGGAATGCGTGGTGTAAGCCGGATTC
TTCTCAAGCTACCTGTCGACTATCCCGTAAAATACACGGACCTTTTCA
CTCACATACACGCCAATGCTGAAGATCTTGTCAAATGGCTATGGAAAA
CGAATCACCCGGATGCGATTCAAATTCTGATCCTCGGTGTAAATGGCA
AGAAAAAGCACCTGGAATACTTAAGCAAAGCCTGCCAAAAACATCCCG
CTGCGGCTATTGCCGCTTATGCTACTTTGCTGGCAATACATGAANATA
ATGAGTGGCGTAAAGCGCTTGTCAAACTGATT (SEQ ID NO: 1113)
GCCCGCAAGCAACTGGTGCTCAACCTGGTTTCCAGCCCAGGTTCCGGT
AAAACCACCCTGCTGACGGAAACCTTAATGCGCCTGAAAGACAGCGTT
CCGTGCGCGGTTATTGAAGGTGACCAGCAAACCGTGAACGATGCCGCA
CGCATTCGCGCTACCGGCACACCAGCGATTCAGGTGAACACCGGTAAA
GGCTGCCATCTTGACGCACAGATGATTGCCGACGCCGCACCGCGTCTG
CCACTGGACGATAACGGTATTCTGTTTATCGAAAACGTTGGCAACNTC
GTATGCCCGGCCAGCTTCGATCTCGGTGAAAAACACAAAGTGGCGGTG
CTTTCCGTTACCGAAGGTGAAGACAAACCGCTGAAATATCCGCATATG
TTTGCCGCCGCCTCGCTGATGCTGCTCAACAAAGTTGACCTGTTGCCG
TATCTCAACTTTGACGTTGAGAAGTGCATCGCCTGCGCCCGCGAAGTC
AATCCAGAAATTGAAATCATCCTTATTTCCGCCACCAGCGGCGAAGGG
ATGGACCAGTGGCTGAACTGGCTGGAGACACAGCGATGTGCATAGGCG
TTCCCGGCCAGATCCGCACCATTGACGGTAACCAGGCGAAAGTCGACG
TCTGCGGCATTCAGCGCGATGTCGATTTAACGTTAGTCGGCAGCTGCG
ATGAAAACGGTCAGCCGCGCGTGGGCCAGTGGGTACTGGTACACGTTG
GCTTTGCCATGAGCGTAATTAATGAAGCCGAAGCACGCGACACTCTCG
ACGCCTTGCAAAACATGTTTGACGTTGAGCCGGATGTCGGCGCGCTGT
TGTATGGCGAGGAAAAATAATGCGTTTTGTTGATGAATATCGCGCGCC
GGAACAGGTGATGCAGTTAATTGAGCATCTGCGCGAACGTGCTTCACA
TCTCTCTTACACCGCCGAACGCCCTCTGCGGATTATGGAAGTGTGCGG
TGGTCATACCCACGCCATTTTTAAATTCGGCCTCGACCAGTTACTGCC
TGAAAACGTTGAGTTTATCCNCGGTCCGGGTTGCCCGGTGTGCGTACT
GCCGATGGGCAGAATCGACACCTGCGTGGAGATTGCCAGCCATCCGGA
AGTCATCTTCTGTACCTTTGGCGACGCCATGCGCGTGCCGGGGAAACA
GGGATCGCTGTTGCAGGCAAAAGCACGCGGTGCCGATGTGCGCATCGT
CTATTCGCCGATGGATGCGTTGAAACTGGCGCAGGAGAATCCAACCCG
CAAAGTGGTGTTCTTCGGCTTAGGTTTTGAAACCACCATGCCGACCAC
CGCCATCACTCTGCAACAGGCGAAAGCGCGTGATGTGCAGAATTTTTA
CTTCTTCTGCCAGCATATTACGCTTATCCCGACGCTGCGCAGTTTGCT
GGAACAGCCGGATAACGGTATCGACGCGTTCCTCGCGCCGGGCCACGT
TAGTATGGTTATCGGCACTGATGCCTATAATTTTATCGCCAGCGATTT
TCAGCGTCCGCTGGTGGTGGCTGGTTTCGAACCCCTTGATCTACTGCA
AGGCGTGGTCATGCTGGTGGAGCAGAAAATAGCGGCCCACAGCAAGGT
AGAGAATCAGTATCGTCGGGTGGTACCGGATGCCGGTAACCTGCTGGC
GCAACAGGCGATTGCCGATGTGTTCTGTGTCAACGGCGACAGCGAATG
GCGCGGCTTAGGCGTGATTGAATCTTCTGGCGTGCACCTGACGCCGGA
TTATCAACGATTCGATGCCGAAGCACATTTCCGCCCGGCACCGCAGCA
GGTCTGCGATGACCCGCGCGCGCGTTGTGGCGAAGTCTTGACGGGCAA
ATGTAAGCCGCATCAATGCCCGCTGTTTGGTAACACCTGTAATCCTCA
AACCGCGTTTGGTGCGCTGATGGTTTCCTCCGAAGGAGCGTGCGCCGC
GTGGTATCAGTATCGTCAGCAGGAGAGTGAAGCGTGAATAATATCCAA
CTCGCCCACGGTAGCGGCGGCCAGGCGATGCAGCAATTAATCAACAGC
CTGTTTATGGAAGCCTTTGCCAACCCGTGGCTGGCAGAGCAGGAAGAT
CAGGCACGTCTTGATCTGGCGCAGCTGGTAGCGGAAGGCGACCGTCTG
GCGTTCTCCACCGACAGTTACGTTATTGACCCGCTGTTCTTCCCTGGC
GGTAATATCGGCAAGCTGGCGATTTGCGGCACCGCGAATGACGTTGCG
GTCAGTGGCGCTATTCCGCGCTATCTCTCCTGTGGCTTTATCCTCGAA
GAAGGATTGCCGATGGAGACACTGAAAGCCGTAGTGACCAGCATGGCA
GAAACCGCCCGCACGGCAGGCATTGCCATCGTTACTGGCGATACTAAA
GTGGTGCAGCGCGGCGCGGCAGATAAACTGTTTATCAACACCGCGGGC
ATGGGCGCAATTCCGGCGAATATTCACTGGGGCGCACAGACGCTAACC
GCAGGCGATGTATTGCTGGTTAGCGGTACACTCGGCGACCACGGGGCG
ACTATCCTTAACCTGCGTGAGCAGCTGGGGCTGGATGGCGAACTGGTC
AGCGACTGCGCGGTGCTGACGCCGCTTATTCAGACGCTGCGTGACATT
CCCGGCGTGAAAGCGCTGCGTGATGCCACCCGTGGTGGTGTAAACGCG
GTGGTTCATGAGTTNGCGGCAGCCTGCGGTTGCGGTATTGAAATTTCT
GAATCAGCGCTGCCGGTTAAACCTGCCGTGCGCGGCGTTTGCGAATTG
CTGGGACTGGACGCCCTGAACTTTGCCAACGAAGGCAAACTGGTGATC
GCCGTTGAACGCAACGCGGCAGAGCAAGTGCTGGCAGCGTTACATTCC
CATCCACTGGGGAAAGACGCGGCGCTGATTGGTGAAGTGGTGGAACGT
AAAGGTGTTCGTCTTGCCGGTCTGTATGGCGTGAAACGAACCCTCGAT
TTACCACACGCCGAACCGCTTCCGCGTATATGCTAATAAAATTCTAAA
TCTCCTATAGTTAGTCAATGACCTTTTGCACCGCTTTGCGGTGCTTTC
CTGGAAGAACAAAATGTCATATACACCGATGAGTGATCTCGGACAGCA
AGGGTTGTTCGACATCACTCGGACACTATTGCAGCAGCCCGATCTGGC
CTCGCTGTGTGAGGCTCTTTCGCAACTGGTAAAGCGTTCTGCGCTCGC
CGACAACGCGGCTATTGTGTTGTGGCAAGCGCAGACTCAACGTGCGTC
TTATTACGCATCGCGTGAAAAAGACACCCCCATTAAATATGAAGACGA
AACTGTTCTGGCACACGGTCCGGTACGCAGCATTTTGTCGCGCCCTGA
TACGTTGCATTGCAGTTACGAAGAATTTTGTGAAACCTGGCCGCAGCT
GGCCGCAGGTGGGCTNTACCCAAAATTTGGTCACTATTGCCTGATGCC
ACTGGCGGCGGAAGGGCATATTTTTGGTGGCTGTGAATTTATTCGTTA
TGACGATCGCCCCTGGAGCGAAAAAGAGTTCAATCGTCTGCAAACATT
TACGCAGATCGTTTCTGTCGTCACCGAACAAATCCAGAGTCGCGTCGT
TAACAATGTCGACTATGAGTTGTTATGCCGGGAACGCGATAACTTCCG
CATCCTGGTCGCCATCACCAACGCGGTGCTTTCCCGCCTGGATATGGA
CGAACTGGTCAGCGAAGTCGCCAAAGAAATCCATTACTATTTCGATAT
TGACGATATCAGTATCGTCTTACGCAGCCACCGTAAAAACAAACTCAA
CATCTACTCCACTCACTATCTTGATAAACAGCATCCCGCCCACGAACA
GAGCGAAGTCGATGAAGCCGGAACCCTCACCGAACGCGTGTTCAAAAG
TAAAGAGATGCTGCTGATTAATCTCCACGAGCGGGATGATTTAGCCCC
CTATGAACGCATGTTGTTCGATACCTGGGGCAACCAGATTCAAACCTT
GTGCCTGTTACCGCTGATGTCTGGCGACACCATGCTGGGCGTGCTGAA
ACTGGCGCAATGTGAAGAGAAAGTGTTTACCACTACCAATCTGAATTT
ACTGCGCCAGATTGCCGAACGTGTGGCAATCGCTGTCGATAACGCCCT
CGCCTATCAGGAAATCCATCGTCTGAAAGAACGGCTGGTTGATGAAAA
CCTCGCCCTGACCGAGCAGCTCAACAATGTTGATAGTGAATTTGGCGA
GATTATTGGCCGCAGCGAAGCCATGTACAGCGTGCTTAAACAAGTTGA
AATGGTGGCGCAAAGTGACAGTACCGTGCTGATCCTCGGTGAAACTGG
CACGGGTAAAGAGCTGATTGCCCGTGCTATCCATAATCTCAGTGGGCG
TAATAATCGCCGCATGGTCAAAATGAACTGCGCGGCGATGCCTGCCGG
ATTGCTGGAGAGCGATCTGTTTGGTCATGAGCGTGGGGCTTTTACCGG
TGCCAGCGCCCAGCGTATCGGTCGTTTTGAACTGGCGGATAAAAGCTC
CCTGTTCCTCGACGAAGTGGGCGATATGCCACTGGAGTTACAGCCGAA
GTTGCTGCGTGTATTGCAGGAACAGGAGTTTGAACGCCTCGGCAGCAA
CAAAATCATTCAGACGGACGTGCGTCTAATCGCCGCGACTAACCGCGA
TCTGAAAAAAATGGTCGCCGACCGTGAGTTCCGTAGCGATCTCTATTA
CCGCCTGAACGTATTCCCGATTCACCTGCCGCCANNNNGCGAGCGTCC
GGAAGATATTCCGCTGCTGGCGAAAGCCTTTACCTTCAAAATTGCCCG
TCGTCTGGGGCGCAATATCGACAGNATTCCTGCCGAGACGTTGCGCAC
CTTGAGCAATATGGAGTGGCCGGGTAACGTACGCGAACTGGAAAACGT
CATTGAGCGCGCGGTATTGCTAACACGCGGCAACGTGCTGCAGCTGTC
ATTGCCAGATATTGCTTTACCGGAGCCTGAAACGCCGCCTGCCGCAAC
GGTTGTCGCTCAGGAGGGCGAAGATGAATATCAGTTGATTGTTCGCGT
GCTGAAAGAAACTAACGGCGTGGTTGCCGGGCCTAAAGGCGCTGCGCA
ACGTCTGGGGCTGAAACGCACGACCCTGCTGTCACGGATGAAGCGACT
GGGAATTGATAAATCGGCATTGATTTAACTGCAAATTGCCGGACAGAT
CTGCCTGTCCGGCATACTATTCACGAGGTTTTTTCGGACGATATTTTT
CCGGCAGTTCTGGCACCGGACACTTGTCATCGATGAGATGACGCACGG
TTAAGATCGGATGACGCCACAGCATTCTCGGCCCGGCCCAACGCATAA
TCTGTTTCATCTCTTCACGCTTTGCAGGCTGGTAACAGTGCACCGGAC
ACTGCTTACAGGCTGGTTTCTCTTCGCCGAACACACATTTATCCAGCC
GCTTTTGCGCGTAAACAAACAACGCCTCGTAATGCTCCGGCTCCGCTG
ACGCCTGCGGGCATTTCGCTTGATAAAGATCGATCATTTTTTTAATCG
TCAGTTTTTCACGAGAGATACGCTTGCCGGACATACTGCCTCCACCTC
ATTAAGATGCATTTATATTACAACTTAATCTTAAAGGGCACTATGACT
CTAAAGAAGAAGGGTTAGCCAACCGATACAATTTTGCGTACTTGCTTC
ATAAGCATCACGCAAAAGCTGCAAAACAGCATCTTTCCCGGAACCAGC
ATCAAGAACTCGCCGTTCGCTTCTTCCCCTGAAATGATTAACTCCGGT
ATCATGTGCGCCTTATGTGATTACAACGAAAATAAAAACCATCACACC
CCATTTAATATCAGGGAACCGGACATAACCCCATGAGTGCAATAGAAA
ATTTCGACGCCCATACGCCCATGATGCAGCAGTATCTCAAGCTGAAAG
CCCAGCATCCCGAGATCCTGCTGTTTTACCGGATGGGTGATTTTTATG
AACTGTTTTATGACGACGCAAAACGCGCGTCGCAACTGCTGGATATTT
CACTGACCAAACGCGGTGCTTCGGCGGGAGAGCCGATCCCGATGGCGG
GGATTCCCTACCATGCGGTGGAAAACTACCTCGCCAAACTGGTGAATC
AGGGCGAGTCCGTTGCCATCTGCGAACAAATTGGCGATCCGGCGACCA
GCAAAGGTCCGGTTGAGCGCAAAGTTGTGCGTATCGTTACGCCAGGCA
CCATCAGCGATGAAGCCCTGTTGCAGGAGCGTCAGGACAACCTGCTGG
CGGCTATCTGGCAGGACAGCAAAGGTTTCGGCTACGCGACGCTGGATA
TCAGTTCCGGTCGTTTTCGCCTGAGCGAACCGGCTGACCGGGAAACGA
TGGCGGCAGAACTGCAACGCACTAATCCTGCGGAACTGCTGTATGCAG
AAGATTTTGCTGAAATGTCGTTAATTGAAGGCCGTCGCGGCCTGCGCC
GTCGCCCGCTGTGGGAGTTTGAAATCGACACCGCGCGCCAGCAGTTGA
ATCTGCAATTTGGGACCCGCGATCTGGTCGGTTTTGGCGTCGAGAACG
CGNNNCNCGGACTTTGTGCTGCCGGTTGTCTGTTGCAGTATGCGAAAG
ATACCCAACGTACGACTCTGCCGCATATTCGTTCCATCACCATGGAAC
GTGAGCAGGACAGCATCATTATGGATGCCGCGACGCGTCGTAATCTGG
AAATCACCCAGAACCTGGCGGGTGGTGCGGAAAATACGCTGGCTTCTG
TGCTCGACTGCACCGTCACGCCGATGGGCAGCCGTATGCTGAAACGCT
GGCTGCATATGCCAGTGCGCGATACCCGCGTGTTGCTTGAGCGCCAGC
AAACTATTGGCGCATTGCAGGATTTCACCGCCGAGTTGCAGCCGGTAC
TACGTCAGGTCGGCGACCTGGAACGTATTCTGGCGCGTCTGGCGTTGC
GTACCGCTCGCCCACGCGATCTGGCCCGTATGCGTCACGCTTTCCAGC
AACTGCCGGAGCTGCGTGCGCAGTTAGAAACTGTTGATAGTGCACCAG
TACAGGCGCTACGTGAGAAGATGGGCGAGTTTGCCGAGCTGCGCGATC
TGCTGGAGCGAGCAATCATCGACACACCGCCGGTGCTGGTACGCGACG
GTGGTGTTATCGCATCAGGCTATAACGAAGANCTGGATGAGTGGCGCG
CGCTGGCTGACGGCGCGACCGATTATCTGGAGCGTCTGGAAGTCCGCG
AGCGTGAACGTACCGGCCTGGACACGCTAAAAGTTGGCTTTAATGCGG
TGCACGGCTACTACATTCAAATCAGCCGTGGGCAAAGCCATCTGGCAC
CTATCAACTATATGCGTCGCCAGACGCTGAAAAACGCCGAGCGCTACA
TCATTCCAGAGCTAAAAGAGTACGAAGATAAAGTCCTCACTTCAAAAG
GCAAAGCACTGGCTCTGGAAAAACAGCTTTATGAAGAGCTGTTCGACC
TGCTGTTGCCGCATCTGGAAGCGTTGCAACAGAGCGCGAGCGCGCTGG
CGGAACTCGACGTGCTGGTGAACCTGGCGGAACGGGCCTATACCCTGA
ACTACACCTGCCCGACCTTCATTGATAAACCGGGCATTCGCATTACCG
AAGGCCGCCATCCGGTGGTTGAACAGGTGCTGAACGAGCCATTTATCG
CCAACCCGCTGAATCTGTCACCGCAGCGCCGGATGTTGATTATTACCG
GTCCGAACATGGGCGGTAAAAGTACNTATATGCGCCAGACCGCACTGA
TTGCGCTGATGGCCTATATCGGCAGCTACGTACCGGCGCAAAAAGTCN
AGATTGGCCCGATTGACCGTATCTTTACCCGCGTAGGGGCAGCGGATG
ATCTGGCTTCCGGGCGTTCAACCTTTATGGTGGAGATGACCGAAACCG
CTAATATTCTGCATAACGCCACCGAGTACAGTCTGGTGCTGATGGATG
AGATTGGGCGCGGAACGTCCACTTACGATGGTCTGTCGCTGGCGTGGG
CGTGCGCGGAAAATCTGGCGAATAAGATTAAGGCGTTGACGCTGTTTG
CCACCCACTATTTCGAGCTGACCCAGTTACCGGAGAAAATGGAAGGCG
TCGCCAACGTGCATCTCGATGCACTGGAGCACGGCGACACCATTGCCT
TTATGCATAGCGTGCAGGATGGCGCGGCGAGCAAAAGCTACGGCCTGG
CGGTTGCAGCTCTGGCCGGCGTGCCAAAAGAGGTTNNNNAGCGCGCAC
GGCAAAAACTGCGTGAGCTGGAAAGCATTTCGCCGAACGACGCCGCTA
CGCAAGTGGATGGTACGCAAATGTCTTTGCTGTCCGTACCGGAAGAAA
CCTCGCCTGCAGTCGAGGCACTGGAAAACCTCGATCCGGATTCACTCA
CCCCGCGTCAGGCGCTGGAATGGATTTATCGCCTGAAGAGTCTGGTGT
AATAATAATTCCCGATAGTCTTTTGCTATCGGGAATATTAACGATAAC
TGACGAATCAAATAAAAATACCCTGTATAATAGGAAAGCTTATTTTAC
AGGGTAAAACCATGCCATCTACACGCTATCAAAAAATCAATACCCATC
ACTATCGCCATATATGGGTCGTTGGTGATATTCATGGTGAATATCAGT
TATTACAATCCCGCTTACATCAACTCTCTTTTTTCCCCGAAACCGACT
TACTTATTTCTGTCGGCGATAATATTGATCGTGGGCCGGAGAGTCTTA
ACGTCCTGCGCCTGCTAAACCAGCCCTGGTTTATCTCCGTTAAAGGCA
ACCACGAAGCAATGGCGCTGGATGCGTTCGCGACCGGCGATGGCAATA
TGTGGCTTGCCAGCGGTGGTGACTGGTTTTTCGATTTAAATGATTCAG
AGCAAAAAGAAGCTACAGATCTGTTGCTGAAATTCCATCACCTTCCAC
ATATTATTGAAATCACTAACGACAACATAAAATATGTCATCGCACATG
CAGATTATCCGGGGAGTGAATATCTCTTTGGTAAAGAAATAGCGGAGA
GCGAATTACTCTGGCCTGTTGATCGTGTGCAGAAATCGCTTAATGGCG
AGTTACAACAAATAAACGGCGCTGATTATTTTATATTTGGACATATGA
TGTTTGATAACATTCAGACGTTCGCTAACCAGATTTATATTGATACCG
GATCGCCGAAAAGCGGGCGGCTGTCATTTTATAAAATAAAGTAATGAG
GCCGGGTAAAGCAAAGCCACTACCCGGCACTTTTTATTAGCGCTTACC
TTCCGCCAGCAGCGGCGGGATGCTCGGCACCATTGGCGCGTCATCAAT
ATCTTTCTGCGTCATGCGGAACGCTTCGGGATAATGTTCGCGGCTGGT
ACGGCGCAGCGGTTCGGTATCGCGCCAGGTATAAAGGCAATGCTGGCA
CTGATATACCGTCCAGACATCTTTCACCGGCGATTTCGCCATCACTTC
AATCTGTTCATCGGCACAACGTGGACAAATCATCTTGTTCTCCTTATT
TACGTGCGGCCAGCATAGCGGTCAGTTTTTCAGCCCAGGCTTTGGTTT
CCGGCAAATCCACCACCGGCTGGCTGTAGTGACCACGGTTGTCCGGGG
CGACAGGCGTGGTGGCGTCGATAATCAGCTTGTCGGTGATCCCCGCCG
GGCTTGAGCCAGGGTCGAGTTCCAGCACGGACATATTCGGCAACTGCA
CCAAATCCCCTGCCGGATTTACTTTCGAGGAGAGCGCCCACATCACCT
GCGGCAGGTTGAACGGGTCAACGTCTTCATCGACCATAATCACCATCT
TTACGTAGCCCAGACCGTGCGGCGTGGTCATCGCACGCAGGCCCACCG
CGCGGGCAAAGCCGCCGTAGCGTTTTTTGGTGGAGATAATCGCCAGCA
GGCCGTGGGTGTACATGGCGTTTACCGCCTGCACTTCCGGGAACTCGG
CTTTCAGTTGTTGATACAGCGGCACACAGGTGGCTGGCCCCATCAGGT
AGTCGATTTCGGTCCACGGCATGCCGAGGTACAGCGATTCGAAAATCG
GCCTGGTGCGGTAAGAGACTTTATCGATACGCACCACGGTCATGTTAC
GCCCGCCGGAGTAGTGCCCGCTAAACTCACCGAACGGCCCTTCGATTT
CGCGTTTGCGGCTTTCGATAACCCCTTCGAGGATCACTTCTGAACCCC
ACGGCACATCAAAACCAGTCAATGGCGCGGTGGCGATCGGGTACGGGC
TTTCGCGCAGCGCGCCTGCCATTTCATACTCAGACTGATCGTATTTCA
GCGGCGTGGCGCCCATAAGGGTGATGATCGGATCGTTACCCAGGGTGA
TGGCAATCGGCAGATCTTCACCGCGCTCTTCCGCTTTATGCAGATGCA
GGGCGATATCGTGCATCGGCACCGGTTGCAGGCCGAGCTTACGCTTGC
CCTTCACTTCCATGCGGTAGATACCGACGTTCTGCTTGCCGAAGTTAT
CCGGGTCGAGCGGATCGCGGGAAACCACGCACGCTTTGTCGAGATAGA
AACCGCCATCACCGTCGTTTAAACGAAACAGCGGCAGGATATCGAACA
GATTAATCTCGTCACCATCGACGGTGTTCTGCGCCCAGGCTGGATTGG
CGCGGCGCTCCGGGGCGATCGGGAAGTTATCCCAGCGGCGGATAAACT
CATCAATCTGTTTTTTAACCGGGGTGTTTGGCGGCAGGCCCAAGGAAA
TCGCGTGGTTCTGCCAGGAACCGATGGTATTCATCGCCACGCGGGCAT
CGGTAAAACCGCGAATATTATCAAACCACAGCGCCGGTGCACCATCGC
CGATACGCCCGGTGGCGTTGGCAGCGGCAGCCAGATCCGGTTCCGCGT
TCACCTCTTCGCTGATTTTCAGTAACTGGCCGTGGTCATCAAGCGCCT
GTAAAAAGCTGCGTAAATCATCAAATGCCATTATTCATTCTCCTGAGA
AAAATTCCGGGCCTGCGGCAATCCTTGCCAGCGCCTGGCGTAGGGATG
TTCAAGGCCAAATTGATCCAGCACGCGGGCTACCACGTGGTGGACAAT
GTCATCTACCGTTTCGGGATGGTTATAAAAGGCAGGCATCGGCGGCAC
CATCGCCACGCCCATGCGTGAAAGTGCGAGCATATTTTCGAGATGGAT
GGTGCTAAGCGGCATTTCACGCGGCACCAGCACCAGTTTGCGGCCTTC
TTTGAGCACGACGTCCGCCGCGCGCCCTACCAGGCCATCAGCGTAACC
AGCGCGGATACCGGCGAGCGTTTTCATACTGCACGGAATAACGATCAT
GCCGTCTGTACGAAAGGAACCTGAGGAGATGGTCGCCGCCTGATCCGC
CGGGTTATGGCTGAAGTCAGCGAGGGCAGCAACATCGCGGGCGCTGTA
AGGCGTTTCCAGTTCAATGGTGGTTTTCGCCCACTTCGACATCACCAG
ATGAGTCTCGACATTCGGCATCTCCCGCAGCGCTTGCAGTAATGCCAC
ACCAAGANGCGCACCGGTAGCCCCTGTCATCCCGACGATCAGTTTCAT
TGCTTACTCTCTTAATTGTTCGTATACGAACATTATTATTAAAATACG
CCTCTGTTCTCATTGCGGCAAGCACTGAAAAGTAACAACATCCCCTTC
GTCACTGAAAAACCGCTATGATAGCGGCAGATAGTTTGCCCGGAGTTT
ACATGGCGTTACGAAATAAAGCGTTCCATCAGTTACGACAGCTTTTTC
AGCAGCACACGGCTCGCTGGCAGCACGAGTTACCTGACCTGACCAAAC
CACAGTATGCGGTGATGCGCGCCATTGCTGATAAGCCTGGTATCGAAC
AGGTGGCACTGATAGAAGCAGCAGTCAGCACCAAAGCAACGTTGGCAG
AAATGTTGGCAAGAATGGAGAATCGTGGCTTAGTCAGACGTGAACATG
ATGCCGCTGACAAGCGGCGTCGCTTTGTCTGGCTGACTGCCGAAGGAG
AAAAGATACTTGCTGCGGCGATACCGATTGGTGACAGCGTGGATGAGG
AATTTTTGGGGCGTTTGAGTGCAGAAGAGCAGGAGCTGTTTGTGCAGC
TGGTGCGCAAGATGATGAACACATAGGATGCAAATTGCCGGGTAGGAC
GCTGACGTGTCTTATCCAGGCGACAAAAACACAAGCTTCCCAAACAAA
AGAAAAAGGCCAGCCTCGCTTGAGACTGGCCTTTCTGACAGATGCTTA
CTTACTCGCGGAACAGCGCTTCGATATTCAGCCCCTGCGTNTGCAGGA
TTTCGCGCAAACGGCGCAGGCCTTCAACCTGAATCTGGCGAACACGTT
CACGGGTGAGGCCAATTTCACGACCTACATCTTCCAGTGTTGCCGCTT
CGTACCCCAGCAAACCGAATCGACGTGCCAGTACTTCACGCTGTTTGG
CGTTCAGCTCGAACAGCCATTTGACGATGCTCTGCTTCATATCGTCAT
CTTGCGTGGTATCTTCCGGACCGTTCTCTTTTTCATCGGCCAGGATGT
CCAGCAACGCTTTTTCGGAATCACCACCCAGCGGGGTGTCTACCGAGG
TAATGCGCTCGTTAAGACGAAGCATACGGCTGACGTCATCAACTGGCT
TATCCAGTTGCTCTGCGATCTCTTCCGCACTTGGTTCATGGTCCAGCT
TATGGGACAACTCACGTGCGGTTCGCAGGTAAACGTTCAGCTCCTTTA
CGATGTGAATCGGCAAACGAATAGTACGGGTTTGGTTCATAATCGCCC
GTTCAATCGTCTGGCGAATCCACCAGGTTGCGTATGTTGAGAAGCGGA
AACCACGTTCCGGGTCAAACTTNTCTACCGCACGGATCAGCCCCAGGT
TGCCNTCTTCGATAAGGTCCAGCAACGCCAGACCACGATTGCCATAAC
GGCGGGCAATTTTTACCACCAGACGCAAGTTACTCTCGATCATCCGGC
GGCGAGAGGCGACATCTCCACGCAGTGCGCGACGCGCAAAATAAACTT
CTTCTTCGGCCGTTAACAGTGGTGAATAACCAATCTCACCAAGGTAAA
GCTGAGTCGCGTCCAACACACGCTGTGTGGCTCCCTGCGATAACAGTT
CCTCTTCGGCCAAATCGTTATCACTGGGTTCCTCTTCTACTAAGGCCT
TTTCGTCAAAAACCTCAACTCCGTTCTCATCAAATTCCGCATCTTCAT
TTAAATCATGAACTTTCAGCGTATTCTGACTCATAAGGTGGCTCCTAC
CCGTGATCCCTTGACGGAACATTCAAGCAAAAGCCTGGTTCCGCCGAT
TTATCGCTGCGGCAAATAACGCAGCGGGTTTACGGATTTCCCCTTGTA
ACGAATTTCAAAATGCAAGCGTGTTGAACTGGTTCCGGTGCTACCCAT
GGTTGCTATTTTTTGCCCCGCCTTCACTTCTTGTTGTTCCCGGACCAG
CATTGTGTCGTTATGGGCGTAGGCACTCAGGTAATCATCATTATGTTT
GATGATAATCAGATTACCGTAGCCGCGCAGCGCGTTACCGGCATAAAC
AANGCGGCCATCTGCGGTCGCGATAATTGCCTGTCCTTTGCTGCCTGC
GATATCAATCCCCTTGTTGCCCCCCTCAGAAGCGCCAAAGGTTTCGAT
CACTTTGCCCTCAGTCGGCCAGCGCCAGGTGGAGATAGGCGTACTGGT
TGATGTACTGCTGACAGTCGGCTCGGTTGTGCTTGCTGTTGGTACCGT
TACAGGCGCTGTGACCGTGGTCGCAGTTGGCTTGTTGTTCGGCAACAT
TTTGTTAGCACTCTGTTCACCCGAAGACTCAGAATACGTAATTGTCGG
TTGCGACGCAACAGCAACGGTGGAATTTTGTGCAGGCTTGATCACAAC
TCCTTGCTCTGCTGCGTCGGCCTGGGTAATGGCATTTCCGCCAGTGAT
TGGCGTACCGGAAGCATTACCCACCTGTAAGGTCTGACCGACGTTCAG
CGCGTATGGTGCCTGAATATTGTTGCGCTGAGCAAGGTCACGGAAATC
GTTGCCAGTAATCCAGGCGATATAGAAAAGCGTGTCGCCTTTTTTCAC
GGTATAGGTACTGCCGCTATAACTGCCTTTCGGAATGTTCCCATACTG
ACGGTTATAGACGATGCGTCCGTTTTCCATCTGTACCGGCTGCTGAGC
TACTGGCTGCACTGGCTGGATTTGCGGTTGTTGAGTAGCCTGAATTTG
TGGCTGCTGCACCGGCTGAATTTGCGGTTGCTGCGCTGTAGACGTCGT
CCCCATTTTCGGCGGCGGCGTAATCAACATACCAGAATTGGTATGTGC
AGGCGCATTGCCATTAACGGAGCTGACCGGTGCCGGTGGATTTGAAGT
GTCAGAACAGCCTGCCAGCCATAGCGAAACCAGTGACAAAGCCGCAAT
GCGGCGAACGGTGAATTTTGGGCTTCCCGCGCTCATTTATCCCCCAGG
AAAAATTGGTTAATAACCAGTGACATAATTACCGTGCAAGGCACCATA
CTGAACACTGGAAAAGATGTTCACGATACGCTGACCTGCGGCAAAATA
ACCAGGAAAAATCCAGGTATTTCCTCACGTTTTAAGCCAGCTCACCCT
TCACTAAAGGGACAAAGCGCACGGCCTCCACGGTATCGATAATAAATT
CGCCTCCCCGACGACGCACCCGTTTCAAATACTGGTGCTCCTCCCCTA
CGGGTAAGACGAGAATCCCGCCTTCGTCCAGCTGCGTCATTAGCGCAG
TTGGAATTTCCGGCGGTGCCGCCGTAACAATGATAGCGTCAAACGGCG
CACGTGCCTGCCAACCTTGCCATCCATCGCCATGACGGGTTGAAACAT
TATGTAAATCAAGATTTTTCAGGCGGCGACGCGCCTGCCACTGCAAGC
CTTTAATCCGTTCAACCGAGCAAACATGCTGGACAAGATGCGCCAGGA
TTGCCGTTTGATATCCCGAACCGGTGCCAATTTCCAGCACCCGCGACT
GCGGCGTCAGCTCGAGTAATTCGGTCATTCGCGCCACCATATACGGCT
GCGAAATCGTCTGCCCCTGACCTATCGGCAAGGCGATATTGTCCCAGG
CTTTTTGTTCAAACGCTTCATCAACGAATTTTTCACGCGGCACGGCGG
CAAGTGCATTCAGCACCTGCTCATCCTGAATACCTTGCGCACGTAATT
GATCCAGAAGTGCTTGTACGCGTCTGCTTACCATTGCGTGCCAACTCC
CACGCTGTTTAACCAGTCTGAAACCACATCTTGCGCGCTATGCGCAGT
TAAATCCACATGCAGCGGCGTGATGGAGACATAGCCCTCATCTACCGC
AGCAAAATCGGTCCCCGGACCGGCATCACATTTACCGCCCGGCGGGCC
AATCCAGTACAGCGTATTGCCGCGCGGATCTTGCTGCGGGATCACCTG
ATCTGCCGGATGTCGTGTACCGCAGCGCGTCACGCGAATACCTTTGAT
TTGATCCAAGGGTAAATCCGGAACATTAATATTAAGAATACGCCCGGT
GCGCAGCGGCTCTTTACACAGTGCGCGCAAAATTGAACAGGTTACCGC
CGCGGCAGTGTCGTAATGTTTATGCCCGTCAAGCGAGACGGCAAGCGC
CGGAAAACCTAAATGACGGCCTTCCATCGCGGCGGCTACCGTACCGGA
ATAAATAACATCATCCCCCAGATTCGGCCCGGCGTTAATTCCGGACAC
AACAATGTCCGGGCGCGGACGCATCAGAGCATTCACGCCAAGATAGAC
GCAATCGGTCGGGGTTCCCATTTGCACAGCAATATCACCATTTTCAAA
GGTAAACGTGCGCAGGGAGGATTCCAGTGTCAGAGAATTTGAAGCGCC
GCTGCGGTTACGATCGGGGGCGACCACCTGAACGTCAGCAAACTCACG
CAAGGCTTTCGCCAGCGTTTGTATACCGGGTGCATGTACCCCGTCATC
ATTACTCAGCAATATGCGCATAATCACCTGTTGTGTTGATAAGTTCCC
TGACAACGCTGGTTGCAAAACTACCCGCCGGAAGCCAGAAACGGATTT
CTACGGTGACGTCATCCCACCAATTCCAGCTTAATTGTTGCGGATACA
GCAGCATCGCTCTGCGCGCGGCTTCAACTTTTTCGCGCACCAGTAAAG
CTTGTAATTCAGTTTCTGCGGCGAGAGCTGCCTGTTCGAATGCCAGCG
CTTCACGCTGAGTTCCCCATTCACCACTGCCCGGCAATGCGGCGGTTA
TCATCAGCTCTTTATCGTTGACGCGACGCTGTAATTCCACCAGTTCTT
CGGTGGTTGCAACAAACCAGCTACCACGTCCGGCTAATTGTAGCGCAT
CGCCGTCAACAACTTGATTAACGTCTGCTTTTTTGAGGCGCTCAGCAA
CAATCTGATTAAACAACGCACTGCGGGCTGCCGACAACCAAAAACTCC
GTTTATTGCGATCGCGCACCGGAGTATTGGTTTGCGCCCAGCGCAGCG
CGCCCTGCAAGTTGCTACCGCCAATCCCAAAACGTTGGGCACCGAAGT
AGTTCGGTACACCTTTTACGCAAATATCGATCAGACGTTGTTCAACGT
CATCGCGATTGCTCACTTCGCGCAGAACCAGGGTAAAGGCGTTACCTT
TCAGCGCCCCCAAACGCAGCTTGCGCTTGTGCCGCGCATACTCCAGCA
CCTGGCAGCCTTCCAATTGAAAGGCGCTCAGATCGGGCATTTCCTTGC
CCGGCACGCGAGCGCATAACCACTGTTCTGTAACAGCATGTTTGTCTT
TTTGCCCAGCAAAGCTGACTTCACGGGCATGAATTTTCAGGAATTTCG
CCAGTGCATCCGCCACAAAACGGGTATTGCAGCCGTTTTTGAGGATTC
TAACCAGAATATGCTCACCTTCACCATCAGGCTCAAAGCCCAAATCTT
CCACCACCACAAAGTCTTCCGGATTGGCTTTCAGCAGCCCGGTGCCTT
GCGGTTTACCGTGGAGGTAAGTGAGATTATCAAACTCAATCATTTTGT
TGCCTTAATGAGTAGCGCCACCGCTTCACAGGCAATCCCTTCCCCACG
TCCGGTAAATCCAAGTTTTTCCGTAGTAGTGGCTTTCACGTTAACATC
ATCCATATGGCAGCCGAGATCTTCGGCAATAAACACGCGCATTTGTGG
AATGTGCGGCAACATCTTCGGTGCCTGAGCGATGATAGTGACATCGAC
GTTGCCAAGGGTATAACCCTTCGCCTGAATACGACGCCAGGCTTCGCG
TAGCAGCTCGCGGCTATCGGCACCTTTAAATGCCGGATCGGTATCCGG
GAACAGTTTGCCGATATCCCCCAGCGCCGCCGCGCCAAGCAATGCATC
GGTCAACGCATGGAGCGCCACGTCGCCATCAGAATGCGCCAGCAATCC
TTTTTCGTAAGGAATGCGTACGCCACCAATGATAATTGGGCCTTCACC
GCCAAAGGCGTGTACGTCAAAACCGTGTCCAATTCGCATTATGTATTC
TCCTGATGGATGGTTCGGGTGAGGTAAAACTCGGCCAGTGCCAAATCC
TCCGGGCGCGTGACTTTAATGTTATCCGCACGGCCTTCGACCAACTGA
GGATGGAATCCGCAATATTCCAGCGCCGAGGCTTCGTCGGTAATAGTC
GCGCCTTCATTTAGAGCGCGCGTCAGACAGTCATGTAACAGCTCACGA
GGGAAAAATTGCGGCGTCAGCGCGTGCCATAAGCCGTTGCGATCAACG
GTATGAGCAATGGCATTTTTGCCCGGTTCGGCACGTTTCATAGTATCG
CGCACTGGTGCGGCTAGGATCCCTCCCGTGCGGCTGGTTTCGCTCAAC
GCCAACAATCGCGCGAGGTCATCCTGATGCAGACAAGGACGAGCGGCG
TCATGCACCAATACCCACTGCGCGTCGCCAGCGGCTTTCAAACCTGCC
AGCACGGAATCGGCACGCTCATCACCGCCATCTACAACGGTGATTTGC
GGATGATTCGCCAGAGGAAGTTGTGCAAAACGGCTATCGCCAGGACTT
ATGGCAATGACGACACGTTTCACCCGGGGATGCGCCAGCAGCGCATAC
ACCGAGTGTTCAAGAATGGTTTGATTACCGATTGAGAGATATTGCTTA
GGACATTCCGTTTGCATTCGACGGCCAAATCCGGCCGCCGGAACCACG
GCGCAAACATCCAAATGAGTGGTTGCCATGTTAATTCCCGGGCTGATT
TATCGATTGTTTTGCCCCGCAGACTGTGCGCGCTTCGACGCGTCAGGC
ACCAGACGATAAAAAGTTTCGCCCGGCCTGGTCATGCTGAGTTCATTA
CGCGCACGCTCTTCGAGCGCCTCCTGGCCGCCATTGAGATCGTCAATT
TCGGCAAAAAGTTGATCGTTTCGCGCTTTAAGTTTCGCGTTTGTAGCT
TGCTGTGCCGCCACATCATCATTGACGCGGGTATAGTCATGTATACCG
TTCTTACCGAACCACAGCGAATACTGTAGCCAGACCAGAATAGCCAGC
AACAGCAGCGTTAGTTTACCCATCCTGCCCCCTGAAAAACGGCATCAT
CATCCCATGCATCCGAAGACGACTCTACATCCTCTGTTGGGGATACCG
CGACAACGCGGGCAAATGTACCACATTTGTCCATTGTTACGTATACCC
AGGGCGTGCAGAACATAATCTCATTATTAGTTACGGTTTGAATTATGA
ACAGAGGAGACAAGAAAGTACAAATTAGCCCAGTAGCCACATAAACAG
TGCGCCAAACATAATGCCTACTGTCATCAGGGTGAAAACAATACTGTA
GCGTAGCTTTCCGTCCATCAATGAATGCAGCGCAATCCCCACCACTAC
CGCAACGGGCATCAGCGCCAGAAAGAAAGGCCAGGTGTAGATAAAGAA
GAACAGCGTGTTAGAGCCATAAATCAACATCGGCATCGCCAGCGCAAA
TAACCAGGAGATAAAACCGACCACGGCACCAGGCAGTGACCATGTGGT
TTCTTCATCCTCAGTAAGGCTGTCGTTATTTGTTAGTGTAATGTTATG
GCTATTACGCATATTTGATCCTGTTACTTTGACGAACCGGGCATGGAA
ACCCGGTGGTGTCTCAGGATCTGATAATATCGTTCTGTCTCAACAGAT
CTAATAATTGCTGTACCAAATTTGTTACTAATTGTTCACCATTGAGAT
GAATTTCTGCCGATTCAGGCGCTTCGTAAACGGAATCTATTCCCGTAA
AGTTGCGCAGTTCACCGGCACGCGCTTTCTTATATAAGCCTTTTGGAT
CGCGGGCTTCGCAAATCGCCAGCGGCGTATCGACAAACACTTCGATAA
AGCGCCCTTCTCCTACGCGTTCGCGAACCATCTGGCGTTCGGCGCGGT
GTGGCGAGATAAATGCGGTCAGCACCACCAGTCCGGCTTCAACCATCA
AATTCGCCACTTCACCGACGCGACGGATATTCTCTTTACGATCGGCAT
CGCTAAAACCGAGATCGCTGCATAATCCGTGGCGAACATTGTCGCCAT
CCAGCAGATACGTACTGACGCCGAGTTTATGTAACGCCTCCTCCAGCG
CCCCAGCGACCGTTGATTTACCGGACCCGGAGAGGCCGGTAAACCACA
GCACTACACCACGATGACCGTGGTGTAGCTCGCGTTGTTGCACAGTGA
CCGGATGGCTATGCCAGACGACGTTTTCGTCATGCAGCGCCATTATTT
CTCCCCCAGCAAATCGCGNNNNCCCCAGTGTGGAAAGTGGCGGCGAAC
CAGGGCATTCAATTCCAGTTCGAATGCACTAAATTCAGATGGCGCAGC
AGTTGCCTGGCTAACTGGCTCATGCACCATACCGGCACCTACGGTCAC
ATTGCTCAGGCGATCGATAAAAATCAGCCCACCGGTAACCGGGTTTTG
CTGATAACGATCTAACACCAGTGGCTCGTCAAAAGTGAGATCCACCAG
GCCGATGCCGTTCAGCGGCAGGTTTTCAACTTCGCGTTGGGTAAGGTT
ATTAATATCAACCTGATAACGAATGCCATCAACACGAGAACGCGTCTT
CTTACCGGCAATTTTGATGTCGTAACTCTGGCCCGGGGAAAGCGGCTG
TTCCGCCATCCATACCACATCCACCGACGCGCTCTGCACAGCTGGTAA
CGCTTCGTCTGCCGCCAGCAGCAGATCGCCACGGCTGATGTCGATCTC
ATCCGTCAGCACCAGGGTGATAGCTTCTCCGGCAAAGGCTTCTTCGCG
ATCACCATCAAAAGTCACGATCCGCGCGACGTTTGATTCCACACCAGA
GGGCAGCACTTTTACACGTTGCCCGACTTCCACGCGACCGGATGCCAG
CGTTCCGGCGTAGCCACGAAAATCGAGATTTGGGCGGTTAACGTACTG
CACCGGGAAGCGCATTGGCTGGGCATCCACCACTCGCTGAATCTCCAC
GGTTTCCAGCACTTCGAGCAGTGTCGGACCGCTGTACCACGGCATACT
TTCACTTTGCGAAGCCACGTTGTCACCTTCCAGTGCGGAGAGCGGCAC
AAAGCGGATATCCAGATTACCCGGCAGCTGCCCGGCAAAGGTCAGATA
ATCTTCACGAATACGGGTGAACGTCTCTTCACTGTAATCCACCAGATC
CATTTTGTTGATCGCCACGACCAGATGTTTGATCCCCAACAGTGTGGA
GATAAAACTGTGACGACGGGTTTGATCGAGCACGCCTTTACGGGCATC
GATCAGTAAGATCGCCAGTTCACATGTCGATGCGCCAGTCGCCATATT
GCGGGTGTACTGCTCGTGCCCTGGAGTGTCGGCGATAATAAATTTACG
CTTCTCGGTAGAGAAATAGCGGTAGGCCACGTCAATGGTGATGCCCTG
TTCACGCTCAGCTTGCAGGCCGTCCACCAGCAGAGCCAGATCCAGCTT
TTCGCCCTGGGTGCCGTGACGCTTACTGTCATTATGCAGCGATGAGAG
CTGATCTTCATAGATTTGGCGGGTATCGTGCAGCAGACGACCAATCAG
GGTACTTTTGCCGTCATCGACGCTACCACAGGTCAGAAAACGCAGCAG
GCTTTTATGTTGTTGCGCAATCATCCAGGCTTCGACGCCGCCTTCATT
GGCGATTTGTTGTGCAAGTGCGGTGTTCATCTTAAAAATACCCCTGAC
GTTTTTTCAGCTCCATAGAACCAGCTTGGTCGCGGTCAATCACGCGCC
CCTGACGTTCACTGGTGGTGGAAACCAGCATCTCTTCGATGATCTCCG
GCAGCGTTTGTGCATTTGACTCCACCGCACCGGTCAGCGGCCAGCAGC
CCAGCGTACGGAAACGCACCATCCGTTTTTTAATCACTTCGCCCGGTT
GCAGGTCGATACGGTTGTCATCAATCATCATCAACATACCGTCGCGTT
CCAGAACCGGACGTTCCGCAGCGAGATACAGCGGCACAATGTCGATAT
TTTCCAGCCAGATGTATTGCCAGATATCCTGCTCGGTCCAGTTAGAGA
GCGGGAAAACGCGGATGCTTTCGCCTTTGTTAATCTGCCCGTTATAGT
TGTGCCACAGCTCCGGGCGCTGATTTTTCGGGTCCCAGCGATGGAAGC
GATCACGGAAAGAGTAGATACGCTCTTTAGCACGGGATTTCTCTTCGT
CGCGGNGCGCACCACCGAAGGCGGCATCAAAACCGTATTTATTCAGCG
CCTGCTTCAGGCCTTCGGTCTTCATAATATCGGTATGTTTCGCGCTGC
CGTGCACGAATGGATTAATCCCCATCGCCACGCCTTCCGGGTTTTTAT
GCACCAGCAGCTCGCAGCCGTAGGCTTTCGCCGTACGATCGCGGAACT
CATACATCTCACGGAATTTCCAGCCGGTATCGACATGCAGCAACGGGA
AAGGCAGCGTACCTGGATAAAACGCCTTGCGCGCCAGATGCAGCATGA
CGCTGGAATCTTTACCGATAGAGTAGAGCATCACCGGATTTGAGAATT
CTGCCGCCACCTCGCGAATAATGTGGATGCTTTCCGCCTCCAGTTGCC
GCAGGTGAGTAAGTCGTATTTGATCCATAACCGTTCCTTTGCAATACC
GCTATTTTCTTGCCATCAGATGTTTCGACTATAGGGAGCGTAAGAGAA
CGAATGAAATTACCAATTAGAATGAGTAGTTCCTTAACGGAATAACGA
TTTGGCAAAGCTAATATCAAAAAGTGCTTAAGGCACCGGATTTCGGGC
GTTTAGGAAGATTTGAAATTGTTTTAGCGCAGCGGCAGTTTCATACTA
TGGCGGTAAAAAAATTTGCATGGTATTTAAGGACTCACTATGTTTTCC
GCATTGCGCCACCGTACCGCTGCCCTGGCGCTCGGCGTATGCTTTATT
CTCCCCGTACACGCNTCGTCACCTAAACCTGGCGATTTTGCCAATACA
CAGGCGCGACATATTGCCACTTTCTTTCCGGGACGAATGACCGGAACA
CCCGCAGAAATGTTATCTGCCGATTATATTCGGCAACAGTTTCAGCAA
ATGGGTTACCGCAGTGATATTCGTACGTTTAATAGCCGATATATTTAT
ACCGCCCGCGATAACCGCAAAAACTGGCACAACGTGACGGGAAGTACG
GTGATTGCCGCTCATGAAGGCAAAGCGCCGCAGCAGATCATTATTATG
GCGCATCTGGATACCTATGCCCCGCAGAGCGACGCAGATGCAGATGCC
AATCTCGGCGGGCTGACGTTACAAGGAATGGATGATAACGCCGCAGGT
TTAGGTGTCATGCTGGAACTGGCAGAACGCCTGAAAAATACGCCTACC
GAGTATGGTATTCGATTTGTGGCGACCAGTGGAGAAGAGGAAGGGAAA
TTAGGCGCTGAGAATTTACTCAAGCGGATGAGTGACACCGAAAAGAAA
AATACGCTGCTGGTGATTAATCTCGATAACTTAATTGTTGGCGATAAA
TTGTATTTCAACAGCGGTGTAAAAACCCCTGAAGCAGTAAGGAAATTA
ACGCGCGACAGGGCGCTGGCAATTGCGCGTAGTCATGGAATTGCCGCA
ACGACCAATCCGGGTTTGAATAAAAATTATCCGAAAGGCACTGGATGT
TGTAATGACGCAGAAATATTCGACAAAGCGGGCATTGCTGTACTTTCG
GTGGAAGCGACAAACTGGAATCTTGGGAATAAAGATGGTTATCAGCAA
CGCGCAAAAACAGCCGCATTCCCTGCGGGAAATAGCTGGCATGACGTA
AGACTGGATAATCAGCAACATATTGATAAAGCACTTCCTGGAAGAATA
GAACGTCGCTGCCGTGACGTTATGCGGATAATGCTACCGCTGGTGAAG
GAGTTGGCGAAGGCGTCTTGATGGGTTGGAAAATGGGAGCTGGGTGTT
CTACCGCAGGGGCGGGGAATTCTAAGTGATATCCATCATCGCATCCAG
TGCGCCCGGTTTATCCCCGCTGATGCGGGGAACACAGCGGCACGCTGG
ATTGAACAAATCCCTGGGCCGGTTTATCCCCGCTGGCGCGGGGAACAC
TTTATACACGGATCCTGTGTGCCGTGGACCGCCGGTTTATCCCCGCTG
GCGCGGGGAACACCACAAACCGCCCATCTTCCCGATTACTGCAGCCGG
TTTATCCCCGCTGGCGCGGGGAACACACTAAGCATACATATCTGTTTT
TAAACAAATTTATTCCACATCAACAATCTACCAACTAAATTCAAACAT
TTCCTTATTTTTAAAGAACACATAACCTATTGATTATCAACAGGAAGA
AAAGAAACCAAACGTAACCCATCCAAATCCACCGGAATACGTCTGTTT
TCTCCCCAGGTCTGAAATTCAAAACCCGACTCGGTATTGGTCGCCCAG
GCCATCACCACATTTCCGCAACCAGCCAGTTGGGTAATTTGCTGCCAG
ATCATCTCCCGAATACGTTTTGATGTATCACCAACATACACACCGGCA
CGCACTTCCAGTAGCCAGATTGCGAGCCGTCCACGTAAGCGCGGAGGG
ACATTTTCTGTAACAACCACGACCATGCTCATCCGCCGCGCCCCCGGT
GACCACTATCACCCAGCGTTTCAGGTTCAGGGATGGCAGGCGGTAACA
TATCCGGCGCGGGTTGTGGTGGTTCAATTTCACCTGCAGCAAGGACTT
CCTCAATTAACGGTATTAATTTGCCCGTTAACTTAGTGCTACGGAAAA
TATCGCGACAGGCTAATCTGACTTCTTTATCAGGTTCTGCGGGTTGCC
TCGCTGCTATTTCAAATGCCTTTGGCACAACCGAATCAAATTTAATGA
TATCGGCTATGTCATAAACAAATGAAAGCGGTTTGCCACTATGAATAA
ATCCAATAGCGGGCGCATATCCCGCGGCTAATACTGCCGCTTCAGAAA
TACCGTACAGACATGATGTGGCAGCACTGATGCAGCGATTCACAACAT
CGCCTTTTTCCCAGTCTTTAGGATCGTATTTGCGACCATTCCATTTCA
CACCATATTGTTTCGCCAGTAATGCATAGGTCTGGCGAACGCGGGATC
CCTCAATTCCCCGTAGCTGATCCACTGAACAGCGAGCTGGCGGTGGCT
CACGAAAACGTAATTCATACATTTTGCGCACCACCTTCAGGCGTAGAT
CTTCCGTTAAAGCCAGCTTTGCCTGGTAGAGTAATTTATCTGCCCGCG
CCCCTCCGGGTTGTCCGGAAGAGTAAACGCGAACGCCCGCTTCACCGA
CCCAGACCAGCAGTGTTCCCACCGTGGCGGCCAGATGCACCGCCGCGT
GGGAAACTCTCGTTCCCGGTTCGAGCATAATGCAGGCGACCGATCCCA
CCGGAATGTGCGTGCGGATCCCGGTTTTGTCGATCAGCACGAAAGCGC
CGTCCAGTACGTCGATTTGACCGTACTGGAGGAAGATCATAGAGGTGC
GATCTTTTAACGGGATCGGACTCAGTGGTACAAACGTCACACCTCTGC
TCCGGGTTTGATCAGCATCAGACCACAGCCGAACGCGCGGCTTTTGCC
ATACCCCTGACTGAGACGTTGCAGAAATAACCCGGGATCGGTGACCGT
AAGCATCCCCGTATAATCCACGCTACTGAACTGGATCAGTTGCCGGGA
GTTTTCCCGCCGCAGTTGCTGTTGTCGGTAGGCATCAACCGAGGTATC
CAGCAGTGTAAAACCGCTTCTCTCCCCCTGCGCTGCCAGCCAGTCCAG
CGCCGCCTGTTGTTGATGCAACCAGACATCACTTCCTTCCGCCTGCCC
CCTCACCTGCCGTTTCGCCTCCATCAGCAGATCGTGGCGCTTGCCCGC
TTTACAGATTGTTGGATTCGCCCGCAGGTTGAAACAAAGTTGTTGTCC
GGTACGCAGCTCGGGCACAAATGACCGGCATTCGATGGTGAACGTTTC
ACTTTCCGCCGGGCGTTCCTGCGACAGTACAAAAAAGCGAAACGCGCC
CTGGAGTTCTTCCCGACGATAAAGAAATTGCCTTTCTTTGCCGCCAGG
GAAGAGATCCCACAGCCACTGATGCATCACATATTCCCCGCGATCCAC
CAAATGCAGCAACTGCGCAGGCGAAAGCTGGCCGGTATGTAAGGTTAT
TCTTGAGAGGTACATGGTTCCTCCTTGCTGAGCCACGGCCCCTGATTG
ATGGTGCGTTCCCCAAACAGCCACTGCTGACGATTTAAAGGAACATCT
CGGCGACGTAATATTTTGCTCKCGACCAGGCCGTCGTGTTCCCCTTCC
CACCAGCATTCATCCTGAAGTTTCGGGAGTGAGACTTTCAGTTCGCGG
AAACTATCCTGATACTGTTGGTATGCGTTACGTAAGACATCAGACGCG
TTGCCTTCGAGCAGTAACGGCGCAAGTGGTAACGCCAGCGGATGACTT
TTTCGCCCCAGATAAAGCGGAAAAACCGGATGACGTAAACCGTCCTGC
AACTGTTCAAGGCTGTAAGGCGCATCGGGGGTTGTTGCCACCGCCACC
ATCCACCAGGCATCGGTGTAGTAGTCGCGCCGGGAGATAATCGCGCTC
AGAAGATCAGGGGCGCTCAACTCTTCGCGACGGCTGAAATAACGCGCT
TTACGCACCTCTTTTGGCATCTGGACCGTGTGATAATCCCGTGCCCAG
CGCGGGTTACGGCTGGCGCAAACCACCAGTGAATAGTGGCGGTTAAAC
GCGTTTAATCGTTCGGTATCATCACGCCGAATCCCTACCCCGGCAGCC
AGCAGCCCCAGCAATGCTGAGCGCGAAGGCAGTTCATGGGTATGACGC
ACTTCGCCGGGGGCATCGACGCCCCAGGATGCCATTGGCCCATGAAGC
TGAAAAATCAAATATTGGCTCATTAGCCGCCCCTTACGCGCAGATAAA
GTCCAGCACGTCCTTCATGCTTCCCTGTTTGTTCATCACGTCAAAGCT
TGCGCATTCGGTCTTCTGTTCATAGACCGTATTCATATTTTCGCGAAG
CGTTGTAATACGCTGCACCGCCACATCTAACTGCCGGGTGCCATTAAT
GGGTTCATAGAAAGCCGCCGCCAGAGAACGTGGTTGTTCGGTGCCTTT
TTCTGCCAGCGCCCAGGAGGCGTAGGCACGGCTGGCAAAGCTGTTCTG
TTTGCCGGTTGGGGAGACTTTAAGTGCGGCTTCCGTAAAGGCGCGCAA
GGTCTGATTAGCTAACGCTTCGTCACCGCCGAGGTTTTCGACCAGCAG
ATCTTTATCGATGCAGATATAGGTGTAGAACAGCGCAGAACCGAATCC
GGTTTCACCAAGATGCCCGGCACCGGCATCTTCAGAAGCCTGGCGCAA
ATCATCAACGGCGGTGAAAAAATCATCTTCGACAATCGTTTCACTGAC
ACCAAATGCATGCGCGACCTGGCAGGCGGCTTCAACATTAAATTCGGG
TTTATTCGCCAGCATACGACCAAACATAGCGATATCTACCGCCATGCG
ATCTTTACGTAACAAAGCGAGATCTTCCTCTTTTGGCGCGCGCTTTTC
TTCGGCCAGTTGATGGGCCAGCGCTNTTACGGCGTCANATTCTGCCGG
GCTGATATGGACTAATTGTTCAGTTTCGGCGTTAGTGAGCGGATCTTT
TGGTTTTTTGTCGTTTTTAGCTTTCCCAAGATAATCCGCAATTTTTGC
CGCCCATTCGATGGCTNTTTTCTCTTCGATGCCTTTCTCAATCAGGAT
AGTTGCCGCCTCACGCGCAATACGCCCACTGCGAATACCAATATGGCC
CGCCAGTGCCTGTTCAAAAAGTGCAGAAGTGCGCCACGCACGTTTCAG
ACTTTGCGAGGAAACGCGCAGTCGCGTTGCTCCACCCAGGACCACGGT
TTTCGGCGCTCCGGTGTCATCACGGTTAAGGTTGGCTGCAGGGTAAGC
GGTTAACAAATGAAGCTGAATAAATGTCGTCATGAGATAGTCCTTTAT
TGATTTTGTTCGTTATCGACGTCGCCAGCCTGGTAATATTCCAGCGCC
CAGCGGATACGGATAAATTCGGTTGGCCGCTGATGACGGCGGTGATGA
TTGAGCAGATCGTCGCTCTCCTGGCACCAGCGGAAGACACCCTCTGCC
AGAGAGTCAAGATTAACGGAACCGTTTAATAATCTGACTGCGCGACGT
AACTGGCGCAGTAATTCATCCGGTGTTTTTACTGCCGACAGGCGAGTA
AAACGCCCCTTTGACATTACCGCCGCCAGCTGCGCAGCAAAAGGCAGC
CGTTCATCAATGGCTTTAACATTAGCGCTAAGTGCGGCTATCAGCGCC
AGTGCGGTAATACGCCATTCAGGTTCATCCTGCCACTTTATTTCTCTG
TTCTTCAGAAACAGGCGAAATCCATCCGTCAGACAAACATCATTAACC
GTCGTACTACGCCGCAGACTGGCACGTTCGCCGCGTTTCTCCTGCAAT
TCCTCATGCCATTTGCGCAGCGTGGCTTTGTGCTCCTCTTTTACAATA
CTCATTCAGCAGCCTCCTGCTTTTTCTCCCTGGCGGCTTTAGCACTTT
GCTTCTCCGCCGATGTTGTAAAATATTTCTTGCGCNCGGTCATGACGC
GTTCCAAATCAACGGGCTCATAAGGATTGGTGAATACACGCTCGTCAA
AATCCTGACGTGCGAATAACCAAATTTCCTTTTGCCATTTGCCGAGTA
ATTCATCCGCATCCTGACCTTCTTCAATTTGGCGCACTAACCTCAGGA
AGCGATGCTGAGTTTTGTTCCAGAAGTCGATATCCACAAAACTGAAAT
CACCCCTTGCACCTTTTGGATCGGAGAACCATGCTTCTTTCAATGCAC
TCCGTAACAGACTCAGAATCCGTGAAGCCGTTTGCGCAGCCAGCCGCA
GCTTCGGTATCTGGCCTTCTTTTTTATTGAGCAGCAGCGGGAAATGGT
GTTCGTACCAACAGCGCGCTTTCATGTTGTCGAAATCATAACCAAATC
CCCACAGGCCCACTTTTGCCTGTTTCAGACTGCTGGCATTAAAGAGTT
TCACCACCAGCGCGGGAAGTTCCGTATTGTTTTCTGACTTACCCGTTT
CGATAAGGCCTAACCAGTCGCGCCAGATTAAACCGCCCGGTTGTGGTT
TAACGGAGTAAAACTCACCGCCCTCTTTAAGTGGTACACGGTAAGGCG
TTAAGGGATGCTGCCACATGGCATAATTCGCACCGTAATTTTTGGTAG
TCATCAAACTCAGAAGCGCGTCACTCTGCTCACCGCAAATATCGCAGT
TGCCGACTGTCGTGGTATTAAAATCAATACGAATACGCCGCGGCATTC
CCCAGTACGCCTGGAGTTTATTGACCTGATCATCGGTTACCACCGCAC
CGGCCAGTTCGCTGGTACGCGTCGGGCCAAGCCAGGGGAAAACCAGAT
CGTCAAATTTTTTGGGTAGCGGTAAGTCGGCTTCATCCTGCGGCATCA
CGTTGAGCCACAGTTTGCGCCACAAGGGGGCTTGTTGATTGCCCTGAT
ACTCCTGCAATTCAATCAGAGTCGTCATCGNCCCACCGCCGCGTAAAC
CGGTGCGATAGCCTTTGCCACCTGACGGCACATTTAACTGTAGGGAGA
ACAGAGCTAACGCAGAACAATGAGAGCATACGTGTTCAGTCACGCCAC
GCTTAATAAAGTGGTCTTTATTAAACTTCGTTGTTTGAGCGCCGGGAA
TCTCAGGCAGTAGCGAAGCGACCTGAACTTTATCGCCCATGAGCACCT
CGAAATCCTGCATAAATGAAGGTGAATCTGGGCCAAACTGGAAAGCGT
GTTCTAATGACAGCAATGCTTCCCGTAGCTTTTCAGCTTCCAGCCCGT
CTTCCCAGATATCATCCCAACGACGATAATCTTTTGGCGCGAAACTGC
TTTGTAGTAACCCCAGCAAAAACTGCCATGCCGCCCCCTGGAGATCTG
CCCGCGGCGCAGCGATATCGACAACATTTTCATCCGCCAGATCGACTG
GCGCCAGCTTGCCTGTTGTTCCGTCTTTAAAACGAACGGGCAACCACG
GGGTTGTCAGAAGTGAAAACGAGTTCATATGTATCCTCTCGTAAAAAA
GACCATCCCTGGTCGGTTGTCCATCATTCCATCCTTTTCGGTGTATCA
ACTATTCAGAGAGTCCTGTCGCAGGTATCTCAATCAACCTTGCCAATC
AATCCCTCCCTGGCCGAATACCCGCAACTTTCGTCATCAGTGACTAAA
ATCACGTTTGCCATTTCCGGATCTTGCCGCTGTTCAATGCACCACTGC
CTGAACGCTTCCCCTTCCAGTAAAGAAAACTCATCCCGATGTTTTTTC
CACCAGCTTCGACGCACTCTGACAACGCTCATTTCCCATGCGTGAGCA
CCGGTGGCATAAGGCTTCACCACACCGGCAATACAGGTAGCCAGCCAC
AGGGAAACAGATTCCTCAGCCAGACGTGTCGACAGCTTTTCCGGAAGG
TAATCGTTGATATTGGCGGCATAGCCAGGCTTGAAGTTCAGGACAAAC
TTTTTAGCCATTGCGCGATCGCAGTAATATTTGCCCACTTGCTCCTGC
TCGCTGCGGGCAAATCCTTCCGGCATTACCACGTCCTCACCGTAGACT
GATTCAATAAGAAGGCGGGCTGCGTGTGGCATTTGAATAGCGCCTTGC
TCACGCAGTACACGCTGCGTCAGCCAGATTCGTCCATGATCGGGATAG
ACATATGCACTGTTACGCATGGCACTGCCGAACCATTCGTCACCAGGA
GCGTCGTCCCAGACGGGGGCCAGAATCAGCAATTCAGGAGGGGAACGC
TCGTCTTTTCCGTCACGCTTTAACTGACCATTAATATCGCGGATATGC
CGCTGTAATCGCCCCGCTCGCTGAATCAGCAAATCAACAGGGGCCAGG
TCGGAGATCATTTCGTCCAGGTCACAATCAACGCTCTGCTCTAAGACC
TGAGTACAAATGAGGACTTTTCCGGCACGCTGTGAACCGTCTTCTTTA
CCAAAGCGTGCCAGCGTCTCCATTTCAATTCGCTGGCGATCGCTAAAA
GCAAAGCGGCTATGAAAGAGTGAAAGGCTGGAAGCGGGAATGACGCCG
CGGGCAAGCAGCTGACGATGAACCTTAATAGCGTCATCGACAGAATTC
CGGATCCAGGCGATGCATTTTCCCTGACTTACCGCCGATTCGATACGC
GCAATACTCTCTTGTTCACTATGAAGCCAACCCACGCTGACGCTACGC
TCAACGTCTTTGCGCGTCGCTACCCGGTGTGAGTTCACATCGGATTTC
GTGACATGCGTCAGCCAGGGGTAATCATCCTTTTCAAGGAACGGAGCT
TCTTGCTGGCCCTCTGTGCCACGCGCAAAGGCGGCGACGAGTTTGTCG
CGCTGCTGTTGGGATAACGTAGCAGAAAGCAAAATGACGCAGTTTCCG
CCACGCGCCTGCCGCTCGATCAGCCCTTCAAGAATGCACGACATGTAA
GCATCACAGGCATGGATCTCATCAGCCAGCAGGATTTTGTTACTCAAC
CCCAGAAGCCGCAGATTATTATGTTTAAACGGCATCACTGCCATCATC
GCCTGATCCAGCGTGCCGACGCCAATTTCAGCCAGTAGCGCCTTCTTG
TTACTGTTGGCAAACCAGGCCGCACATCCCTGACTGAATGTTTGTTCA
TCCGGTTCTTCTGACCCGACTAAATCACCGGACCAGAGTGATTCATTG
AAGCGGTCCATTAATGTGCGGGCACTGTGTGCCAGCACCAAGCTGGGG
CGGGACTCTGGCGAATAGAAAGCAAGCCAGGTTTTGACCAGCCGATCG
TACATGGCATTGGCCGTTGCCATTGTTGGCAGGCCAAAAAACAAACCC
TGTGCTTTCCTCGCAGCCATCAACCTGTGCGCCAGGATAAGCGCCGCT
TCTGTTTTACCTGCGCCAGTCACGTCTTCCAGAATAAATAACTGTGGC
CCTGGCTGGCTGATATCCAGATCCAGTACCTTTTGCTGTAATGGTGTC
GGGTGCTCAATAAAAGGAAACAGCGTATTAATTCCGGTGAAAGGTGCG
GTTTCTGCTTTTGGAGGAAAGACGGTTAAGGCGTTTTGAGCCTGAACT
AAAGTTTTCTGCCAGTAATCTTTAATATCCATTGGGTGTGCGACGCGT
GGAAAAAATCGCGTTGACGAACCCGTCCAGTCTGCGAGTACGACTGTT
GCAGAGATATACCAGGAAAGTTGTTTTAAAAGTTCAACGCCCTCGTCA
TCATCCCAGAATGTGGGAATCTCTATGAGCGGAAACAGTGCCTTGATT
TCAAGGAGAAAATCTCGCGCGGCAGCTTTGTCTTCAGGCAGAAAATTA
TCCAGCTCATCAATACGGTCAGGTGGTCGACCATGATGCCCGGTAGTT
ATGGACATCCACATCTCTATTACACGTGTAAGTTTACGAGAAGAGAGT
GAAGATGAAGGAAGCAACTCCTCACATTCACTTAAATAATAATTCCAC
AGCCAGTAACCCAGCGTTGAATGAGAGATCTTTTCGTAATTCTTTCTG
GAACCTTCCGGAATCTTGAGTTCAGGGGCCAGGTAAAGTTGCTGAAAA
GAGCGGGCAAATTTTCCAATATCGTGCCAGCACAGCAACCAAGCGAAA
AATTGAGCCGCCTGTTCCTTGTCAGAAATCCCTAATTGACGAAAGTAA
TCAGCCAGCCCGAAGCAATTTCTTTTAACCATTAAATAGCCCATTGCG
GCCACATCCAGCGAATGCCAGCAAAGAAGGTGATAGCCGTCGCCACCC
TCTTTCTCGCCACGTCGGGTTTTTCCCCAGAAATCAAAGAAAGTCACA
ATATTTTTATCCTTCAGTAAACTTAAAGGATATTTACGCATATTTTAA
AAATTATCTGTGATATATATCAGCCAATAAACATTTATATTATGAATA
AATTAATGATTTTCATTTGAAATTCATAATGATAAATAGAGACTATAT
ATTCACATAAANNA (SEQ ID NO: 1461)

While the foregoing specification teaches the principles of the present claimed embodiments, with examples provided for the purpose of illustration, it will be appreciated by one skilled in the art from reading this disclosure that various changes in form and detail can be made without departing from the spirit and scope of the invention. These methods are not limited to any particular type of nucleic acid sample: plant, bacterial, animal (including human) total genome DNA, RNA, cDNA and the like may be analyzed using some or all of the methods disclosed in this invention. This invention provides a powerful tool for analysis of complex nucleic acid samples. From experiment design to detection of E. coli O55:H7 assay results, the above invention provides for fast, efficient and inexpensive methods for detection of pathogenic E. coli O55:H7.

All publications and patent applications cited herein are incorporated by reference in their entirety for all purposes to the same extent as if each individual publication or patent application were specifically and individually indicated to be so incorporated by reference. Although the present invention has been described in some detail by way of illustration and example for purposes of clarity and understanding, it will be apparent that certain changes and modifications may be practiced within the scope of the appended claims.

### REFERENCES

The following references, to the extent that they provide exemplary procedural or other details supplementary to those set forth herein, are specifically incorporated herein by reference:
1: Leopold S R, Magrini V, Holt N J, Shaikh N, Mardis E R, Cagno J, Ogura Y, Iguchi A, Hayashi T, Mellmann A, Karch H, Besser T E, Sawyer S A, Whittam T S, Tarr P I. A precise reconstruction of the emergence and constrained radiations of Escherichia coli 0157 portrayed by backbone concatenomic analysis. Proc Natl Acad Sci USA. 2009 May 26; 106(21):8713-8.
2: Wick L M, Qi W, Lacher D W, Whittam T S. Evolution of genomic content in the stepwise emergence of Escherichia coli O157:H7. J. Bacteriol. 2005 March; 187(5): 1783-91.
3: Zhang Y, Laing C, Steele M, Ziebell K, Johnson R, Benson A K, Taboada E, Gannon V P. Genome evolution in major Escherichia coli O157:H7 lineages. BMC Genomics. 2007 May 16; 8:121.
4: Iguchi A, Ooka T, Ogura Y, Asadulghani, Nakayama K, Frankel G, Hayashi T. Genomic comparison of the O-antigen biosynthesis gene clusters of Escherichia coli O55 strains belonging to three distinct lineages. Microbiology. 2008 February; 154(Pt 2):559-70.
5: Tarr P I, Schoening L M, Yea Y L, Ward T R, Jelacic S, Whittam T S. Acquisition of the rfb-gnd cluster in evolution of Escherichia coli O55 and 0157. J. Bacteriol. 2000 November; 182(21):6183-91.
6: Feng P C, Monday S R, Lacher D W, Allison L, Siitonen A, Keys C, Eklund M, Nagano H, Karch H, Keen J, Whittam T S. Genetic diversity among clonal lineages within Escherichia coli O157:H7 stepwise evolutionary model. Emerg Infect Dis. 2007 November; 13(11):1701-6.
7: Laing C R, Buchanan C, Taboada E N, Zhang Y, Karmali M A, Thomas J E, Gannon V P. In silico genomic analyses reveal three distinct lineages of Escherichia coli O157:H7, one of which is associated with hyper-virulence. BMC Genomics. 2009 Jun. 29; 10:287.
8. Perna, N. T., et al., (2001) Nature 409(25):529-533

### Patent Applications

Polymorphic loci that differentiate Escherichia coli O157:H7 from other strains. US 2002/0150902 A1.
Detection of pathogenic bacteria. US 2004/0110251 A1.

What is claimed is:

1. An isolated nucleic acid sequence selected from the group consisting of nucleic acid sequences having SEQ ID NO:66, SEQ ID NO:252, SEQ ID NO:1113, SEQ ID NO:1461, fragments thereof, at least 25 nucleotide sequences thereof and complements thereof.

2. An isolated nucleic acid sequence comprising at least 90% nucleic acid sequence identity to the isolated nucleic acid sequence of claim 1.

3. An isolated nucleic acid sequence of claim 1 comprising SEQ ID NO:66, fragments thereof, at least 25 nucleotide sequences thereof, complements thereof and nucleic acid sequences comprising at least 90% nucleic acid sequence identity thereof.

4. An isolated nucleic acid sequence of claim 1 comprising SEQ ID NO:252, fragments thereof, at least 25 nucleotide sequences thereof, complements thereof and nucleic acid sequences comprising at least 90% nucleic acid sequence identity thereof.

5. An isolated nucleic acid sequence of claim 1 comprising SEQ ID NO:1113, fragments thereof, at least 25 nucleotide sequences thereof, complements thereof and nucleic acid sequences comprising at least 90% nucleic acid sequence identity thereof.

6. An isolated nucleic acid sequence of claim 1 comprising SEQ ID NO:1461, fragments thereof, at least 25 nucleotide sequences thereof, complements thereof and nucleic acid sequences comprising at least 90% nucleic acid sequence identity thereof.

7. An isolated nucleic acid sequence selected from the group consisting of nucleic acid sequences having SEQ ID NO:1, SEQ ID NO:2, SEQ ID NO:3, SEQ ID NO:4, SEQ ID NO:5, fragments thereof, at least 25 nucleotide sequences thereof, complementary sequences thereof and sequences comprising at least 90% nucleic acid sequence identity thereof.

8. An isolated nucleic acid sequence of claim 7 selected from the group consisting of nucleic acid sequences having SEQ ID NO:1, fragments thereof, at least 25 nucleotide sequences thereof, complementary sequences thereof and sequences comprising at least 90% nucleic acid sequence identity thereof.

9. An isolated nucleic acid sequence of claim 7 selected from the group consisting of nucleic acid sequences having SEQ ID NO:2, fragments thereof, at least 25 nucleotide sequences thereof, complementary sequences thereof and sequences comprising at least 90% nucleic acid sequence identity thereof.

10. An isolated nucleic acid sequence of claim 7 selected from the group consisting of nucleic acid sequences having SEQ ID NO:3, fragments thereof, at least 25 nucleotide sequences thereof, complementary sequences thereof and sequences comprising at least 90% nucleic acid sequence identity thereof.

11. An isolated nucleic acid sequence of claim 7 selected from the group consisting of nucleic acid sequences having SEQ ID NO:4, fragments thereof, at least 25 nucleotide sequences thereof, complementary sequences thereof and sequences comprising at least 90% nucleic acid sequence identity thereof.

12. An isolated nucleic acid sequence of claim 7 selected from the group consisting of nucleic acid sequences having SEQ ID NO:5, fragments thereof, at least 25 nucleotide sequences thereof, complementary sequences thereof and sequences comprising at least 90% nucleic acid sequence identity thereof.

13. An isolated nucleic acid sequence selected from the group consisting of nucleic acid sequences having SEQ ID NO: 6, SEQ ID NO:7, SEQ ID NO:8, SEQ ID NO:9, SEQ ID NO:10, SEQ ID NO: 11, SEQ ID NO:12, SEQ ID NO:13, SEQ ID NO:14, SEQ ID NO:15, SEQ ID NO:16, SEQ ID NO: 17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, fragments thereof, at least 10 contiguous nucleotide sequences thereof, complements thereof and labeled derivatives thereof.

14. An isolated nucleic acid sequence comprising at least 90% nucleic sequence identity to the isolated nucleic acid sequence of claim 13.

15. A method of distinguishing an E. coli O55:H7 from a non-O55:H7 E. coli strain comprising: detecting at least one of a nucleic acid sequence selected from the group consisting of SEQ ID NO: 66, SEQ ID NO: 252, SEQ ID NO: 1113, SEQ ID NO: 1461, SEQ ID. NO: 1, SEQ ID NO:2, SEQ ID NO:3, SEQ ID NO:4, SEQ ID NO:5, fragments thereof, and complements thereof, wherein detection of one of the at least one nucleic acid sequences identifies E. coli O55:H7.

16. The method of claim 15, wherein detecting the at least one nucleic acid sequence comprises at least one technology selected from the group consisting of amplification, hybridization, mass spectrometry, nanostring, microfluidics, chemiluminescence, enzyme technologies and combinations thereof.

17. The method of claim 16, wherein amplification is selected from the group consisting of polymerase chain reaction (PCR), RT-PCR, asynchronous PCR (A-PCR), and asymmetric PCR (AM-PCR), strand displacement amplification (SDA), multiple displacement amplification (MDA), nucleic acid strand-based amplification (NASBA), rolling circle amplification (RCA), transcription-mediated amplification (TMA).

18. The method of claim 15, further comprising isolating nucleic acid from a sample.

19. The method of claim 18, wherein the sample is a food sample, an agricultural sample, a produce sample, an animal sample, an environmental sample, a biological sample, a water sample and an air sample.

20. A method for detecting Escherichia coli O55:H7 in a sample comprising the steps of:
a) providing an isolated nucleotide sequence of an E. coli O55:H7-specific nucleotide sequence selected from the group consisting of SEQ ID NO: 66, SEQ ID NO: 252, SEQ ID NO: 1113, SEQ ID NO: 1461, SEQ ID NO:1, SEQ ID NO: 2, SEQ ID NO: 3, SEQ ID NO: 4, SEQ ID NO: 5, fragments thereof, at least 25 nucleotide sequences thereof, complements thereof and sequences comprising at least 90% nucleic acid sequence identity thereof;
b) contacting the isolated nucleotide sequence with the sample; and
c) detecting hybridization of the nucleotide sequence to a complementary nucleotide sequence in the sample.

21. A method for detecting Escherichia coli O55:H7 in a sample comprising the steps of:
a) identifying at least a first target nucleic acid sequence specific to E. coli O55:H7;
b) hybridizing at least a first pair of polynucleotide primers to the target nucleic acid sequence;
c) amplifying the first target nucleic acid sequence to form a first amplified target nucleic acid sequence product; and
d) detecting the at least first amplified target nucleic acid sequence product, wherein detection of the at least first amplified target nucleic acid sequence product is indicative of the presence of E. coli O55:H7.

22. The method of claim 21 further comprising:
a) identifying a second target nucleic acid sequence specific to E. coli O55:H7;
b) hybridizing a second pair of polynucleotide primers to the second target nucleic acid sequence;
c) amplifying the second target nucleic acid sequence to form a second amplified target nucleic acid sequence product; and
d) detecting the second amplified target nucleic acid sequence product, wherein detection of the second amplified target nucleic acid sequence product is indicative of the presence of E. coli O55:H7.

23. The method of claim 22 wherein the first target nucleic acid sequence specific to E. coli O55:H7 and the second target nucleic acid sequence specific to E. coli O55:H7 are selected from the group consisting of SEQ ID NO: 66, SEQ ID NO: 252, SEQ ID NO: 1113, SEQ ID NO: 1461, SEQ ID NO:1, SEQ ID NO: 2, SEQ ID NO: 3, SEQ ID NO: 4, SEQ ID NO: 5, fragments thereof, at least 25 nucleotide sequences thereof, complements thereof and sequences comprising at least 90% nucleic acid sequence identity thereof.

24. The method of claim 22 wherein the first primer pair and the second primer pair are selected from a group consisting of SEQ ID NO: 6, SEQ ID NO:7, SEQ ID NO:8, SEQ ID NO:9, SEQ ID NO:10, SEQ ID NO: 11, SEQ ID NO:12, SEQ ID NO:13, SEQ ID NO:14, SEQ ID NO:15, SEQ ID NO:16, SEQ ID NO: 17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, fragments thereof, at least 10 contiguous nucleotide sequences thereof complements thereof, and labeled derivatives thereof.

25. The method of claim 22, wherein the detecting comprises using a primer selected form the group consisting of SEQ ID NO: 6, SEQ ID NO:7, SEQ ID NO:8, SEQ ID NO:9, SEQ ID NO:10, SEQ ID NO: 11, SEQ ID NO:12, SEQ ID NO:13, SEQ ID NO:14, SEQ ID NO:15, SEQ ID NO:16, SEQ ID NO: 17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, fragments thereof, at least 10 contiguous nucleotide sequences thereof complements thereof, and labeled derivatives thereof.

26. A method for distinguishing a bacteria from an E. coli O55:H7 comprising analyzing the genome of the bacteria for the presence of a sequence selected from the group consisting of SEQ ID NO:1, SEQ ID NO:66, SEQ ID NO:2, SEQ ID NO:252, SEQ ID NO:3, SEQ ID NO:4, SEQ ID NO:1113, SEQ ID NO:5 and SEQ ID NO:1461, fragments thereof, at least 25 nucleotide sequences thereof and sequences comprising at least 90% nucleic acid sequence identity thereof by the method of claim 21.

27. The method of claim 26, wherein the bacteria is a Salmonella Sp.

28. The method of claim 26, wherein the bacteria is an E. coli O157:H7 or an E. coli O26:H11.

29. The method of claim 26, wherein the bacteria is a Shigella spp.

30. The method of claim 29, wherein the Shigella spp. is selected from the group consisting of Shigella dysenteriae, Shigella flexneri, Shigella boydii and Shigella sonnei.

31. The method of claim 30, wherein the Shigella dysentaeria is a strain selected from the group consisting of strain 1012, strain M131649 and strain Sd197.

32. The method of claim 30, wherein the Shigella flexneri is a strain selected from the group consisting of strain 2457T, strain 301 and strain 8401.

33. The method of claim 30, wherein the Shigella boydii is a strain selected from the group consisting of strain BS512 and strain Sb227.

34. The method of claim 30, wherein the Shigella sonnei is a strain selected from the group consisting of strain 53G and strain Ss046.

35. A kit for the detection of E. coli O55:H7 comprising:
at least one pair of PCR primers designed from a group of nucleic acid sequences consisting of SEQ ID NO:1, SEQ ID NO: 2, SEQ ID NO: 3, SEQ ID NO: 4, SEQ ID NO: 5, SEQ ID NO: 66, SEQ ID NO: 252, SEQ ID NO: 1113, SEQ ID NO: 1461, fragments thereof, complementary sequences thereof, sequences comprising at least 90% nucleic acid sequence identity thereof and complementary sequences comprising at least 90% nucleic acid sequence identity thereof; and
at least one probe designed from a group of nucleic acid sequences consisting of SEQ ID NO:1, SEQ ID NO: 2, SEQ ID NO: 3, SEQ ID NO: 4, SEQ ID NO: 5, SEQ ID NO: 66, SEQ ID NO: 252, SEQ ID NO: 1113, SEQ ID NO: 1461, fragments thereof, complementary sequences thereof, sequences comprising at least 90% nucleic acid sequence identity thereof and complementary sequences comprising at least 90% nucleic acid sequence identity thereof.

36. The kit of claim 35, further comprising one or more components selected from a group consisting of: at least one enzyme, dNTPs, at least one buffer, at least one salt, at least one control nucleic acid sample and an instruction protocol.

37. The kit of claim 35, wherein the probe is labeled.

38. The kit of claim 35, wherein at least one primers of the PCR primer pair is a labeled primer.

39. A kit for the detection of E. coli O55:H7 comprising:
at least one pair of PCR primer selected from a group of nucleic acid sequences consisting of SEQ ID NOs: 6, SEQ ID NO:7, SEQ ID NO:8, SEQ ID NO:9, SEQ ID NO:10, SEQ ID NO: 11, SEQ ID NO:12, SEQ ID NO:13, SEQ ID NO:14, SEQ ID NO:15, SEQ ID NO:16, SEQ ID NO: 17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, fragments comprising at least 10 contiguous nucleotide sequences thereof, complements thereof and labeled derivatives thereof; and
at least one probe selected from a group of nucleic acid sequences consisting of SEQ ID NOs: 6, SEQ ID NO:7, SEQ ID NO:8, SEQ ID NO:9, SEQ ID NO:10, SEQ ID NO: 11, SEQ ID NO:12, SEQ ID NO:13, SEQ ID NO:14, SEQ ID NO:15, SEQ ID NO:16, SEQ ID NO: 17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, fragments comprising at least 10 contiguous nucleotide sequences thereof, complements thereof and labeled derivatives thereof.

40. The kit of claim 39, further comprising one or more components selected from a group consisting of: at least one enzyme, dNTPs, at least one buffer, at least one salt, at least one control nucleic acid sample and an instruction protocol.
